(19) United States
(12) Patent Application Publication
(10) Pub. No.: US 2026/0102265 A1
(43) Pub. Date: Apr. 16, 2026

(54) POLYMER STENT FOR BELOW THE KNEE TREATMENT OF PERIPHERAL ARTERY DISEASE

(71) Applicant: Abbott Cardiovascular Systems, Inc., Santa Clara, CA (US)

(72) Inventors: Jennifer M. Jones-McMeans, Oakland, CA (US); Karine S. Ruster, San Jose, CA (US)

(21) Appl. No.: 19/355,338

(22) Filed: Oct. 10, 2025

Related U.S. Application Data
(60) Provisional application No. 63/706,176, filed on Oct. 11, 2024.
(60) Provisional application No. 63/896,299, filed on Oct. 9, 2025.

(51) Int. Cl. A61F 2/958; A61B 6/50; A61B 8/06; A61F 2/06; A61L 31/06; A61L 31/08; A61L 31/10; A61L 31/14; A61L 31/16

(52) U.S. Cl. CPC A61F 2/958; A61B 6/507; A61B 8/06; A61L 31/06; A61L 31/08; A61L 31/10; A61L 31/148; A61L 31/16; A61F 2002/068; A61F 2210/0004; A61F 2250/0098; A61L 2300/204

(57) ABSTRACT

Disclosed are methods of treating peripheral artery disease (particularly chronic limb threatening ischemia (CLTI)) with implantation of a polymeric scaffold into a vessel located below the knee. After implantation into a blood vessel of the patient below the knee, efficacy of the procedure is measured by performing duplex ultrasound (DUS) and/or angiography to measure binary restenosis at the site of the target lesion, where binary restenosis is defined as the presence of a hemodynamically significant restenosis of >50% identified through angiography, or peak systolic velocity ratio (PSVR) ≥2.0 as identified by DUS. The scaffold is made from a bioresorbable polymer. The scaffold has a pattern of interconnected elements. The interconnected elements include a plurality of rings connected by links, wherein each ring includes struts and crowns, and the struts are configured to fold at the crowns when the scaffold is crimped to a balloon.

### CROSS REFERENCE TO RELATED APPLICATIONS

This application claims the benefit of and priority to U.S. Provisional Application No. 63/706,176, filed on Oct. 11, 2024, entitled, “POLYMER STENT FOR BELOW THE KNEE TREATMENT OF PERIPHERAL ARTERY DISEASE”, as well as U.S. Provisional Application No. 63/896,299, filed on Oct. 9, 2025, entitled, “POLYMER STENT FOR BELOW THE KNEE TREATMENT OF PERIPHERAL ARTERY DISEASE”, the entire contents of each of which is hereby incorporated by reference in its entirety.

### BACKGROUND OF THE INVENTION

### Technical Field

The present disclosure pertains generally to implantable medical devices and methods of their use. More particularly, the present invention pertains to polymeric scaffolds that can be expanded, e.g., by a delivery balloon, and their use in below the knee (BTK) treatment of peripheral artery disease (PAD).

### Description of the Related Art

Radially expandable endoprostheses are artificial devices adapted to be implanted in an anatomical lumen. An “anatomical lumen” refers to a cavity or duct of a tubular organ such as a blood vessel, urinary tract, or bile duct. Stents are examples of endoprostheses that are generally cylindrical in shape and function to hold open and sometimes expand a segment of an anatomical lumen (one example of a stent is found in U.S. Pat. No. 6,066,167 to Lau et al, and numerous other examples will be familiar to those of skill in the art). A stent “scaffold” is the portion of the device that is inserted into a blood vessel to expand the vessel and prevent or treat a blockage. Stent scaffolds are typically made of metal mesh, which remains in the body permanently, although more recently there has been some exploration of bioresorbable stents, meaning they dissolve and/or are absorbed by the body over time. The terms “stent” and “scaffold” will be used interchangeably herein. Stents are often used in the treatment of atherosclerotic stenosis in blood vessels. “Stenosis” refers to a narrowing or constriction of the diameter of a bodily passage or orifice. In such treatments, stents reinforce the walls of the blood vessel and prevent restenosis following angioplasty in the vascular system. “Restenosis” refers to the reoccurrence of stenosis in a blood vessel or heart valve after it has been treated (e.g., as by balloon angioplasty, stenting, valvuloplasty, etc.) with apparent success.

The treatment of a diseased site or lesion with a stent involves both delivery and deployment of the stent. “Delivery” refers to introducing and transporting the stent through an anatomical lumen to a desired treatment site, such as a lesion. “Deployment” corresponds to expansion of the stent within the lumen at the treatment region. Delivery and deployment of a stent are accomplished by positioning the stent about one end of a catheter, inserting the end of the catheter through the skin into an anatomical lumen, advancing the catheter in the anatomical lumen to a desired treatment location, expanding the stent at the treatment location, and removing the catheter from the lumen.

In the case of a balloon expandable stent, the stent is mounted about a balloon disposed on the catheter. Mounting the stent typically involves compressing or crimping the stent onto the balloon prior to insertion in an anatomical lumen. At the treatment site within the lumen, the stent is expanded by inflating the balloon. The balloon may then be deflated and the catheter withdrawn from the stent and the lumen, leaving the stent at the treatment site. In the case of a self-expanding stent, the stent may be secured to the catheter via a retractable sheath. When the stent is at the treatment site, the sheath may be withdrawn which allows the stent to self-expand.

The stent scaffold should be able to satisfy a number of basic, functional requirements. The scaffold should be capable of withstanding the structural loads, for example, radial compressive forces, imposed on the scaffold as it supports the walls of a vessel after deployment. Therefore, a scaffold should possess adequate radial strength. After deployment, the scaffold should adequately maintain its size and shape throughout its service life despite the various forces that may come to bear on it. In particular, the scaffold should adequately maintain a vessel at a prescribed diameter for a desired treatment time despite these forces. The treatment time may correspond to the time required for the vessel walls to remodel, after which the stent is no longer necessary for the vessel to maintain a desired diameter.

Radial strength, which is the ability of a stent scaffold to resist radial compressive forces, relates to a scaffold's radial yield strength and radial stiffness around a circumferential direction of the stent. A scaffold's “radial yield strength” or “radial strength” (for purposes of this application) may be understood as the compressive loading, which if exceeded, creates a yield stress condition resulting in the stent diameter not returning to its unloaded diameter, i.e., there is irrecoverable deformation of the stent. When the radial yield strength is exceeded the scaffold is expected to yield more severely and only a minimal additional force is required to cause major deformation.

Even before the radial yield strength is exceeded, there may be permanent deformation in the scaffold following a radial compressive load, but this degree of permanent deformation somewhere in the scaffold is not severe enough to have a significant effect on the scaffold's overall ability to radially support a vessel. Therefore, in some cases the art may view “radial yield strength” as the maximum radial loading, beyond which the scaffold stiffness changes dramatically. Units for “radial yield strength” are sometimes force-divided-by-length, which is an expression of radial yield strength on a per-unit-length basis. Thus, for a radial yield strength per unit length, e.g., F N/mm, the radial load which, if it exceeds this value, would result in significant change in stiffness for a stent having two different lengths, L1 and L2, would therefore be the product F*L1 and F*L2, respectively. The value F, however, is the same in both cases, so that a convenient expression can be used to appreciate the radial yield strength independent of the length of the scaffold. Typically, the radial force that identifies the point where stiffness is lost does not change much on a per-unit-length basis when the scaffold length changes.

Stents implanted in coronary arteries are primarily subjected to radial loads, typically cyclic in nature, which are due to the periodic contraction and expansion of vessels as blood is pumped to and from a beating heart. Stents implanted in peripheral blood vessels, or blood vessels outside the coronary arteries, e.g., iliac, femoral, popliteal, renal, subclavian and other arteries, however, should be capable of sustaining both radial forces and crushing or pinching loads. These stent types are implanted in vessels that are closer to the surface of the body. Because these stents are close to the surface of the body, they are particularly vulnerable to crushing or pinching loads, which can partially or completely collapse the stent and thereby block fluid flow in the vessel.

As compared to a coronary stent, which is limited to radial loads, a peripheral stent should accommodate the significant differences between pinching or crushing loads and radial loads, as documented in Duerig, Tolomeo, Wholey, Overview of superelastic stent Design, MIN INVAS THER & ALLIED TECHNOL 9 (3/4), pp. 235-246 (2000) and Stockel, Pelton, Duerig, Self-Expanding Nitinol Stents Material and Design Considerations, EUROPEAN RADIOLOGY (2003), each of which is herein incorporated by reference in its entirety. The corresponding crushing and radial stiffness properties of the stent also can vary dramatically. As such, the degree of radial stiffness of a stent does not, generally speaking, also indicate the degree of pinching stiffness possessed by the stent. The two stiffness properties are not the same, or even similar.

For example, the amount of cross-sectional crush expected for a peripheral stent implanted within the femoral artery has been estimated to be about 5.8±7%, 6.5±4.9% and 5.1±6.4% at the top, middle and bottom portions of the femoral artery in older patients and 2.5±7.7%,-0.8±9.4% and −1.5±10.5% for younger patients. Other considerations for peripheral stents are the degree of bending and axial compression the stent can withstand without mechanical loss of strength/stiffness. As compared to coronary stents, a peripheral stent usually has lengths of from about 36 to 40 mm when implanted in, for example, the superficial femoral artery. As such, the stent should be ﬂexible enough to withstand axial compression and bending loading without failure. The amount of bending and axial compression expected has been studied and reported in Nikanorov, Alexander, M. D. et al., Assessment of self-expanding Nitinol stent deformation after chronic implantation into the superficial femoral artery, herein incorporated by reference in its entirety.

To date, the most commonly used type of peripheral stents are self-expanding stents made from super-elastic material, such as Nitinol. This type of material is known for its ability to return to its original configuration after severe deformation, such as a crushing load or longitudinal bending. However, with self-expanding stents, the high resiliency of super-elastic material produces what is commonly referred to as a “chronic outward force” (COF) on the blood vessel supported by the stent. Complications resulting from COF are discussed in Schwartz, Lewis B. et al. Does Stent Placement have a learning curve: what mistakes do we as operators have to make and how can they be avoided?, Abbott Laboratories; Abbott Park, Ill., USA, herein incorporated by reference in its entirety. It is believed that a COF exerted on a blood vessel by a conventional self-expending stent is a main contributor to high degrees of restenosis of lesions treated by the self-expanding stent. Indeed, it has been shown that not even an anti-proliferative drug delivered from a drug-eluting self-expandable stent can mitigate the restenosis caused by the stent's COF.

Stents that are plastically deformed by a balloon to support a vessel do not suffer from this drawback. Indeed, balloon-expanded stents, in contrast to self-expanding stents made from a super-elastic material, have the desirable quality of being deployable to the desired diameter for supporting the vessel without exerting residual outward forces on the vessel. However, the prior art has concluded that plastically deformed stents, once collapsed, pinched, or crushed in a peripheral artery will remain so, permanently blocking the vessel. The prior art has concluded, therefore, that plastically deformed stents pose an undesirable condition to the patient and should not be used to treat peripheral blood vessels.

A polymer scaffold, such as that described in U.S. Publication No. 2010/0004735 (herein incorporated by reference in its entirety) is made from a biodegradable, bioabsorbable, bioresorbable, or bioerodable polymer. The terms biodegradable, bioabsorbable, bioresorbable, biosoluble or bioerodable refer to the property of a material or stent to degrade, absorb, resorb, or erode from an implant site. The polymer scaffold described in U.S. Publication No. 2010/0004735, as opposed to a conventional metal stent, is intended to remain in the body for only a limited period of time. The scaffold is made from a biodegradable or bioerodable polymer. In many treatment applications, the presence of a stent in a body may be necessary for only a limited period of time—until its intended function of, for example, maintaining vascular patency and/or drug delivery is accomplished. Moreover, it is believed that biodegradable scaffolds allow for improved healing of the anatomical lumen, as compared to metal stents, which may lead to a reduced incidence of late-stage thrombosis. In these cases, there is a desire to treat a vessel using a polymer scaffold—in particular a bioerodable polymer scaffold—as opposed to a permanent metal stent, so that the prosthesis's presence in the vessel is for a limited duration. However, there are numerous challenges to overcome when developing a polymer scaffold.

Furthermore, among patients with chronic limb-threatening ischemia (CLTI) and infrapopliteal artery disease, angioplasty has been associated with frequent reintervention and adverse limb outcomes from restenosis. The effect of the use of drug-eluting resorbable scaffolds on these outcomes is unknown in the art, although if suitable devices and methods of treatment could be developed, such could prove to be advantageous.

Peripheral artery disease is a global health epidemic estimated to affect more than 230 million people, including 7 to 12 million people in the United States alone. The most severe manifestation is chronic limb-threatening ischemia (CLTI), which is characterized by ischemic rest pain and nonhealing ulceration or gangrene and is associated with a high risk of amputation. The adverse limb outcomes associated with CLTI affect quality of life and life expectancy; the prognosis is worse than that for most cancers.

Open surgical revascularization with saphenous-vein bypass has been shown to increase the likelihood of limb salvage in highly selected patients with CLTI. However, for the treatment of patients with CLTI and infrapopliteal artery disease (i.e., arterial disease below the knee), angioplasty has recently been shown to be superior to surgery. Infrapopliteal angioplasty has limitations, such as elastic recoil, dissection, and restenosis, that reduce the durability of the procedure.

Metal stents can interfere with future reintervention, and are often not particularly appropriate for use below the knee. It would be advantageous to develop viable polymeric stents and associated methods that could be used below the knee, although there are many problems to address, in order for such to be successful. In addition, such potential uses are largely unproven, and there is not only a need for development of appropriate scaffolds, but also a need for evaluating the safety and efficacy of drug-coated resorbable scaffolds for the treatment of infrapopliteal artery disease in patients with CLTI, to ensure that any such procedures would provide improved outcomes, as compared to available alternatives.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. Embodiments of the present invention provide systems, methods, and devices for below the knee treatment of peripheral artery disease (PAD) within a patient's body.

As discussed above, drug-eluting resorbable scaffolds have potential advantages that may make them suitable for the treatment of infrapopliteal artery disease. Drug-eluting resorbable scaffolds have unique scaffolding properties that allow them to overcome mechanical failure, while acting as a delivery platform for an antiproliferative drug during the restenotic phase after intervention. These scaffolds also undergo resorption over time, which facilitates vessel remodeling and potentially reduces the late complications associated with permanent metal stents.

There are a variety of factors that affect a polymeric scaffold's ability to retain its structural integrity and/or shape when subjected to external loadings, such as crimping and balloon expansion forces. These interactions are complex and the mechanisms of action not fully understood. Characteristics differentiating a polymeric, bio-absorbable scaffold of the type expanded to a deployed state by plastic deformation from a similarly functioning metal scaffold are many and significant. Indeed, several of the accepted analytic or empirical methods/models used to predict the behavior of metallic scaffolds tend to be unreliable, if not inappropriate, as methods/models for reliably and consistently predicting the highly non-linear, time-dependent behavior of a polymeric loadbearing structure of a balloon-expandable scaffold. The models are not generally capable of providing an acceptable degree of certainty required for purposes of implanting the scaffold within a body, or predicting/anticipating actual empirical data.

Moreover, it is recognized that the state of the art in medical device-related balloon fabrication, e.g., non-compliant balloons for scaffold deployment and/or angioplasty, provide only limited information about how a polymeric material might behave when used to support a lumen within a living subject via plastic deformation of a network of rings interconnected by struts. In short, methods devised to improve mechanical features of an inflated, thin-walled balloon structure, most analogous to mechanical properties of a pre-loaded membrane when the balloon is inflated and supporting a lumen, simply provide little if any insight into the behavior of a deployed polymeric scaffold. One difference, for example, is the propensity for fracture or cracks to develop in a polymer scaffold. The art recognizes the mechanical problem as too different to provide helpful insights, despite a shared similarity in class of material. At best, the balloon fabrication art provides only general guidance for one seeking to improve characteristics of a balloon-expanded, bio-absorbable polymeric scaffold.

Polymer materials considered for use as a polymeric scaffold, e.g. poly(L-lactide) (“PLLA”), poly(L-lactide-coglycolide) (“PLGA”), polyD-lactide-co-glycolide) or poly (L-lactide-co-D-lactide) (“PLLA-co-PDLA”) with less than 10% D-lactide, and PLLD/PDLA stereo complex, may be characterized, through comparison with a metallic material used to form a stent, in some of the following ways. A suitable polymer has a relatively low strength to weight ratio, which means that more material is needed to provide an equivalent mechanical property to that of a metal. Therefore, struts should be made thicker and wider to have the required strength for a stent to support lumen walls at a desired radius. The scaffold made from such polymers also tends to be brittle or have limited fracture toughness, rather than being ductile. The anisotropic and rate-dependent inelastic properties (i.e., strength/stiffness of the material varies depending upon the rate at which the material is deformed) inherent in the material, only compound this complexity in working with a polymer, particularly a bioabsorbable polymer such as PLLA or PLGA.

Processing steps performed on, and design changes made to a metal stent that have not typically raised concerns for a metal stent may not apply to a polymer scaffold due to the non-linear and sometimes unpredictable nature of the mechanical properties of the polymer under a similar loading condition. It is sometimes the case that one needs to undertake extensive validation before it even becomes possible to predict more generally whether a particular condition is due to one factor or another e.g., was a defect the result of one or more steps of a fabrication process, or one or more steps in a process that takes place after scaffold fabrication (e.g., crimping). Consequently, a change to a fabrication process, post-fabrication process, or even relatively minor changes to a scaffold pattern design should be investigated more thoroughly than if a metallic material were used instead of a polymer. It follows, therefore, that when choosing among different polymeric scaffold designs for improvement thereof, there are far less inferences, theories, or systematic methods of discovery available as tools for steering one clear of unproductive paths, and towards more productive paths for improvement, than when making changes in a metal stent. In other words, there is a significantly higher degree of unpredictability when designing a polymer stent and associated processes.

Applicant recognizes, therefore, that inferences previously accepted in the art for stent validation or feasibility when an isotropic and ductile metallic material was used would be inappropriate for a polymeric scaffold. A change in a polymeric scaffold pattern may affect not only the stiffness or lumen coverage of the scaffold in its deployed state supporting a lumen, but also the propensity for fractures to develop when the scaffold is crimped or being deployed. This means that, in comparison to a metallic stent, there is generally no assumption that can be made as to whether a changed scaffold pattern may or may not produce an adverse outcome or require a significant change in a processing step (e.g., tube forming, laser cutting, crimping, etc.). Simply put, the highly favorable, predictable properties of a metal (generally invariant stress/strain properties with respect to the rate of deformation or the direction of loading, and the material's ductile nature), which simplify the stent fabrication process, allow for inferences to be more easily drawn between a changed stent pattern and/or a processing step and the ability for the stent to be reliably manufactured with the new pattern and without defects when implanted within a living subject, as compared to a polymeric stent. No such predictability exists when designing a polymeric stent, and the applicability of teachings in the art related to metal stents is limited, if applicable at all, due to such differences.

A change in the pattern of the struts and rings of a polymeric scaffold that is plastically deformed—both when crimped to, and when later deployed by, a balloon—unfortunately, is not predictable to the same or similar degree as for a metal stent. Indeed, unexpected problems may arise in polymer scaffold fabrication steps as a result of a changed pattern that would not have necessitated any changes if the pattern was instead formed from a metal tube. In contrast to changes in a metallic stent pattern, a change in a polymeric scaffold pattern may necessitate other modifications in fabrication steps or post-fabrication processing, such as crimping and sterilization.

In addition to meeting the requirements described above, it is desirable for a scaffold to be radiopaque, or fluoroscopically visible under x-rays. Accurate placement is facilitated by real-time visualization of the delivery of a scaffold. A cardiologist or interventional radiologist can track the delivery catheter through the patient's vasculature and precisely place the scaffold at the site of a lesion. This is typically accomplished by fluoroscopy or similar x-ray visualization procedures. For a scaffold to be fluoroscopically visible, it should be more absorptive of x-rays than the surrounding tissue. Radiopaque materials in a scaffold may allow for its direct visualization. However, a significant shortcoming of a biodegradable polymer scaffold (and polymers generally composed of carbon, hydrogen, oxygen, and nitrogen) is that they are radiolucent with no radiopacity. Biodegradable polymers tend to have x-ray absorption similar to body tissue. One way of addressing this problem is to attach radiopaque markers to structural elements of the stent. A radiopaque marker can be disposed within a structural element in such a way that the marker is secured to the structural element. However, the use of radiopaque stent markers on polymeric stents entails a number of challenges. One challenge relates to the difficulty of insertion of the markers. These and related difficulties are discussed in U.S. Publication No. 2007/0156230, herein incorporated by reference.

There is a need, therefore, to develop a prosthesis for treating peripheral blood vessels that possesses the desirable qualities of a balloon-expanded stent, but which does not exert residual outward forces on the vessel (as in the case of a self-expanding stents) while, at the same time, is sufficiently resilient to recover from a pinching or crushing load in a peripheral blood vessel, in addition to the other loading events expected within a peripheral blood vessel that are not typically experienced by a coronary scaffold. There is also a need to fabricate such a polymer scaffold so that the prosthesis also is capable of possessing at least a minimum radial strength and stiffness required to support a peripheral blood vessel, a low crossing profile, and a limited presence in the blood vessel. There is also a need for a scaffold that is easily monitored during its pendency using standard imaging techniques and is capable of high yield production.

An embodiment of the present disclosure provides a polymer scaffold suited to address the foregoing needs including high crush recoverability, e.g., at least about 90-95% after a 50% crushing load. The scaffold can be cut from a polymer tube and crimped to a balloon. Accordingly, the an embodiment of the present disclosure provides a balloon expandable, plastically deformed scaffold cut from a tube and that is suitable for use as a peripheral scaffold. As such, the drawbacks of self-expanding stents can be obviated by practicing the embodiments as described herein.

To date, the art has relied on metals or metal alloys for support and treatment of peripheral blood vessels. As mentioned earlier, once a metallic stent is implanted, it remains in the body permanently, which is not desired, particularly in a peripheral vessel. A scaffold made from a material that dissolves after it treats an occluded vessel, therefore, would be preferred over a metal stent. A polymer, however, is much softer than a metal. If it will serve as a replacement to metal, a new design approach is needed. In addition, earlier work with polymer coronary stents (e.g., ABSORB BVS) exhibited unexpected difficulties in adverse long term patient outcomes, even after the polymer stent had substantially fully dissolved. For example, significant concerns were raised in the form of elevated rate of scaffold thrombosis, target lesion failure and target vessel failure, as compared to conventional drug-eluting stents. As the data included herein shows, such adverse outcomes are not applicable to peripheral vessel placement, such that there are distinct and surprising advantages and improvements in patient outcomes, when using a polymeric bioresorbable stent implanted in a peripheral, below the knee vessel as compared to state of the art treatments, such as angioplasty.

High radial force, small crimped profile, and crush recovery is needed in the polymer scaffold. If the material cannot be modified enough to meet these needs, then a modification to the design of the scaffold network of struts is required. Some exemplary stent geometries that may be suitable for such use are disclosed in U.S. Publication No. 2022/0226133, which is herein incorporated by reference in its entirety.

As will be appreciated, at least some aspects of polymer scaffolds as disclosed herein contradict conclusions that have been previously made in the art concerning the suitability of a balloon-expandable stent or scaffold for use in peripheral blood vessels, particularly as used below the knee. The problems concerning self-expanding stents are described above. Conventional wisdom holds that a balloon expanded stent having sufficient radial strength and stiffness, as opposed to a self-expanding stent, is not a suitable replacement, especially in vessels that will impose high bending and/or crushing forces on the implanted prosthesis. Actual data show that the presently contemplated methods employing balloon-expandable scaffolds surprisingly provide excellent results, as compared to existing state of the art treatments (e.g., angioplasty), for patients suffering from peripheral artery disease (PAD), particularly chronic limb threatening ischemia (CLTI).

According to the present disclosure, crush-recoverable polymer scaffolds possessing particular properties of radial stiffness and strength, fracture toughness and the capability of being crimped down to a target delivery diameter are able to properly balance three competing design attributes: radial strength/stiffness verses toughness, in-vivo performance verses compactness for delivery to a vessel site, and crush recovery verses radial strength/stiffness.

Disclosed herein are embodiments of a scaffold and associated methods that can effectively balance these competing needs, thereby providing an alternative to metal stents that suffer from chronic outward force. As will be appreciated from the disclosure, various polymer scaffold combinations were fabricated and tested in order to better understand the characteristics of a scaffold that might address at least the following needs:
Crush recoverability of the scaffold without sacrificing a desired minimal radial stiffness and strength, recoil, deploy-ability and crimping profile;
Acute recoil at deployment, i.e., the amount of diameter reduction within 30 minutes of deployment by the balloon;
Delivery/deployed profile i.e., the scaffold could advantageously be reduced in size during crimping while maintaining structural integrity;
In vitro radial yield strength and radial stiffness;
Crack formation/propagation/fracture-when crimped and expanded by the balloon, or when implanted within a vessel and subjected to a combination of bending, axial crush and radial compressive loads;
Uniformity of deployment of scaffold rings when expanded by the balloon; and
Pinching/crushing stiffness.

Based on these studies, some embodiments of the present disclosure may include particular relationships characterizing the polymer scaffold relative to crush recoverability. For example, there may be particularly desired values for:
a ratio of outer diameter to wall thickness;
a ratio of outer diameter to strut width;
a ratio of radial stiffness to pinching stiffness;
a ratio of pinching stiffness to scaffold diameter;
a ratio of radial stiffness to scaffold diameter;
a ratio of strut or link thickness to its width; and
a ratio of pre-crimp scaffold diameter to strut moment of inertia.

For additional detail on possible relationships between these material properties and/or scaffold dimensions, see U.S. Publication 20220226133, which is herein incorporated by reference in its entirety.

While principally described in the context of a bioresorbable polymer scaffold, e.g., formed of PLLA, it will be appreciated that other biocompatible polymers may additionally or alternatively be used (e.g., PLGA, PCL, or others). Additional details of potentially suitable bioresorbable polymer materials that could be used in the presently described embodiments are described in U.S. Pat. Nos. 8,206,635 and 11,628,077, each of which is herein incorporated by reference in its entirety. For example, high molecular weight polylactic acid and/or polyglycolic acid polymers (e.g., PLA, PLLA, PDLA, PLGA, or the like) may be dip coated, rather than extruded. Extruding such polymeric materials is of course also possible, e.g., particularly where the molecular weights are more moderate. It will be appreciated that a wide variety of bioresorbable scaffold materials may be suitable for use, so long as the materials are biocompatible, bioresorbed within a reasonable time period (e.g., 5 years or less, 4 years or less, or 3 years or less), and otherwise exhibit the desired physical and mechanical properties. Additional exemplary materials and scaffold examples are described in U.S. Pat. Nos. 7,473,417 and 7,939,611, each of which is herein incorporated by reference in its entirety. For example, such references describe TYROCORE polymeric materials, that can be used in the fabrication of scaffolds that may be used in embodiments as described herein. For example, such polymeric materials may include halogen substituted (e.g., iodine) phenol moieties, within the polymer backbone. In an embodiment, the polymer may include an amino acid (e.g., tyrosine) moiety or derivative thereof which has been substituted with iodine, bromine, or another relatively heavy atom halogen, for increased radiopacity. In another embodiment, the scaffold may be formed from another bioerodable material, such as a bioerodable metal alloy (e.g., a bioerodable magnesium alloy), which may similarly erode or be resorbed within the body within a similar time period (e.g., 5 years or less, 4 years or less, or 3 years or less). For any such bioresorbable polymer, or other bioerodable or bioresorbable scaffold, the scaffold may remain in place, supporting the vessel within which it is implanted for at least 6 months, at least 9 months, or at least 12 months.

In an embodiment, exemplary weight average molecular weight values for an employed PLA, PLLA, PLDA, PLGA or other polymer may be at least 10,000 g/mol, at least 20,000 g/mol, at least 25,000 g/mol, or at least 30,000 g/mol. Such values may range up to 200,000 g/mol, up to 150,000 g/mol, or up to 130,000 g/mol, such as from 30,000 g/mol to 200,000 g/mol, from 40,000 g/mol to 80,000 g/mol, or over 100,000 g/mol, particularly for high molecular weight examples (e.g., where dipping rather than extrusion may be used in at least some steps of manufacture). Exemplary molecular weight values may include 20,000 g/mol, 30,000 g/mol, 40,000 g/mol, 50,000 g/mol, 60,000 g/mol, 70,000 g/mol, 80,000 g/mol, 90,000 g/mol, 100,000 g/mol, 110,000 g/mol, 120,000 g/mol, 130,000 g/mol, 140,000 g/mol, 150,000 g/mol, 160,000 g/mol, 170,000 g/mol, 180,000 g/mol, 190,000 g/mol or 200,000 g/mol. Ranges may be defined by selecting any two of such values. While such values may represent weight average molecular weight values, in another embodiment, such values may be for number average molecular weight values. Determination of molecular weight may be by any suitable technique, e.g., through gel permeation chromatography or other size exclusion chromatography technique.

The present disclosure relates in particular to methods of treatment of peripheral artery disease (PAD) and chronic limb threatening ischemia (CLTI). According to one embodiment, a polymeric scaffold is inserted into a peripheral artery below the knee at the site of a target lesion of a subject, the scaffold having a pattern of interconnected elements. The polymeric scaffold may be bioresorbable. Angiography and/or Duplex Ultrasound (DUS) may be periodically performed after insertion of the scaffold in order to measure binary restenosis at the site of the target lesion, where the presence of a binary restenosis is defined as the presence of a hemodynamically significant restenosis of >50% identified through angiography, or peak systolic velocity ratio (PSVR) ≥2.0 as identified by DUS. According to one aspect of the disclosure, insertion of the scaffold reduces the risk of one or more of (1) an above ankle amputation, (2) a total occlusion of the target vessel, (3) a binary restenosis of the target lesion, or (4) a clinically driven target lesion revascularization (CD-TLR) for at least 1 year after insertion of the scaffold. Preferably, the risk is reduced for at least 3 of the 4 such outcomes. Insertion of the scaffold may additionally be non-inferior relative to balloon angioplasty for preventing (1) a major adverse limb event (MALE), wherein MALE is defined as an above-ankle amputation and/or re-intervention, including thrombosis, within 6 months after inserting the scaffold into the peripheral artery below the knee, and (2) death of the subject within 30 days of inserting the scaffold into the peripheral artery below the knee. Preferably both of such outcomes are prevented.

Another aspect of the present disclosure is directed to a method for treating peripheral artery disease (PAD) below the knee, comprising:
inserting into a peripheral artery below the knee at the site of a target lesion of a subject a scaffold made from a material comprising a polymer, the scaffold having a pre-crimp diameter and a wall thickness such that a ratio of the pre-crimp diameter to the wall thickness is from about 30 to about 60,
the scaffold having a pattern of interconnected elements, the interconnected elements comprising struts and links, and
performing duplex ultrasound (DUS) and/or angiography after inserting the scaffold into the peripheral artery below the knee to measure binary restenosis at the site of the target lesion,
wherein the binary restenosis is defined as the presence of a hemodynamically significant restenosis of >50% identified through angiography, or peak systolic velocity ratio (PSVR) ≥2.0 as identified by DUS.

Another aspect of the present disclosure is directed to a method for treating peripheral artery disease (PAD) below the knee, comprising:
inserting into a peripheral artery below the knee at the site of a target lesion of a subject a scaffold made from a material comprising a polymer,
the scaffold having a pattern of interconnected elements, the interconnected elements comprising struts and links, and
performing duplex ultrasound (DUS) and/or angiography after inserting the scaffold into the peripheral artery below the knee to measure binary restenosis at the site of the target lesion,
wherein the binary restenosis is defined as the presence of a hemodynamically significant restenosis of >50% identified through angiography, or peak systolic velocity ratio (PSVR) ≥2.0 as identified by DUS, and
wherein when the DUS indicates the presence of an abnormal reference peak systolic velocity (PSV), the method further comprising identifying one or more correlating factors, the correlating factors comprising one or more of:
   (1) a focal increase in the absolute peak systolic velocity (PSV) at the site of the target lesion,
   (2) a spectral broadening of the waveform at the site of the target lesion,
   (3) a post-stenotic turbulence (PST) and/or a change in a waveform shape and/or drop in peak systolic velocity distal to the site of the target lesion, or
   (4) a review of one or more B-mode images showing a plaque burden at the site of the target lesion, and
based on the identification of the one or more correlating factors, performing further interventional steps based on the confirmation of binary restenosis.

Another aspect of the present disclosure is directed to a method for treating peripheral artery disease (PAD) below the knee, comprising:
inserting into a peripheral artery below the knee at the site of a target lesion of a subject a scaffold made from a material comprising a polymer,
the scaffold having a pattern of interconnected elements, the interconnected elements comprising struts and links, and
performing duplex ultrasound (DUS) and/or angiography after inserting the scaffold into the peripheral artery below the knee to measure binary restenosis at the site of the target lesion,
wherein the binary restenosis is defined as the presence of a hemodynamically significant restenosis of >50% identified through angiography, or peak systolic velocity ratio (PSVR) ≥2.0 as identified by DUS,
wherein the DUS and/or angiography is performed at one or more of 30 days (+/−14 days), 3 months (+/−14 days), 6 months (+/−28 days), 1 year (+/−28 days), 2 years (+/−28 days), 3 years (+/−28 days), 4 years (+/−28 days), or 5 years (+/−28 days) after inserting the scaffold into the peripheral artery below the knee, and
wherein if the angiography and/or the DUS indicates that there is binary restenosis, the method further comprising performing an interventional procedure, the interventional procedure being selected from angioplasty, implanting of a second resorbable scaffold or a metallic stent, brachytherapy, atherectomy, thrombectomy, intravascular lithotripsy, remedial surgery, genetic treatment with gene transfer or stem cell infusion.

Another aspect of the present disclosure is directed to a method for treating peripheral artery disease (PAD) below the knee, comprising:
inserting into a peripheral artery below the knee at the site of a target lesion of a subject a scaffold made from a material comprising a polymer,
the scaffold having a pattern of interconnected elements, the interconnected elements comprising struts and links, and
performing duplex ultrasound (DUS) and/or angiography after inserting the scaffold into the peripheral artery below the knee to measure binary restenosis at the site of the target lesion,
wherein the binary restenosis is defined as the presence of a hemodynamically significant restenosis of >50% identified through angiography, or peak systolic velocity ratio (PSVR) ≥2.0 as identified by DUS,
wherein the DUS and/or angiography is performed at one or more of 30 days (+/−14 days), 3 months (+/−14 days), 6 months (+/−28 days), 1 year (+/−28 days), 2 years (+/−28 days), 3 years (+/−28 days), 4 years (+/−28 days), or 5 years (+/−28 days) after inserting the scaffold into the peripheral artery below the knee,
wherein when the DUS indicates the presence of an abnormal reference peak systolic velocity (PSV), the method further comprising identifying one or more correlating factors, the correlating factors comprising one or more of:
(1) a focal increase in the absolute peak systolic velocity (PSV) at the site of the target lesion,
(2) a spectral broadening of the waveform at the site of the target lesion,
(3) a post-stenotic turbulence (PST) and/or a change in a waveform shape and/or drop in peak systolic velocity distal to the site of the target lesion, or
(4) a review of one or more B-mode images showing a plaque burden at the site of the target lesion, and
based on the identification of the one or more correlating factors, performing further interventional steps due to the confirmation of binary restenosis.

Another aspect of the present disclosure is directed to a method for treating peripheral artery disease (PAD) below the knee, comprising:
inserting into a peripheral artery below the knee at the site of a target lesion of a subject a scaffold made from a material comprising a polymer, wherein the polymer is bioresorbable,
the scaffold having a pattern of interconnected elements, the interconnected elements comprising struts and links,
wherein the scaffold exhibits a crush recoverability of at least about 90% after a 50% crushing load,
further wherein the scaffold exhibits at least one of a pinching stiffness of at least about 0.5 N/mm, a radial strength of at least about 0.3 N/mm, a wall thickness of at least about 0.008″, or a wall thickness from about 0.008″ to about 0.014″, and
performing duplex ultrasound (DUS) and/or angiography after inserting the scaffold into the peripheral artery below the knee to measure binary restenosis at the site of the target lesion,
wherein the binary restenosis is defined as the presence of a hemodynamically significant restenosis of >50% identified through angiography, or peak systolic velocity ratio (PSVR) ≥2.0 as identified by DUS.

Another aspect of the present disclosure is directed to a method for treating peripheral artery disease (PAD) below the knee, comprising:
inserting into a peripheral artery below the knee at the site of a target lesion in a target vessel of a subject a scaffold made from a material comprising a polymer,
the scaffold having a pattern of interconnected elements, the interconnected elements comprising struts and links, and
performing duplex ultrasound (DUS) and/or angiography after inserting the scaffold into the peripheral artery below the knee to measure binary restenosis at the site of the target lesion,
wherein the binary restenosis is defined as the presence of a hemodynamically significant restenosis of >50% identified through angiography, or peak systolic velocity ratio (PSVR) ≥2.0 as identified by DUS, and
wherein insertion of the scaffold is effective to, relative to balloon angioplasty, reduce the risk of the following for at least one year after insertion of the scaffold:
   (1) a total occlusion of the target vessel,
   (2) a binary restenosis of the target lesion, and
   (3) a clinically driven target lesion revascularization (CD-TLR).

Another aspect of the present disclosure is directed to a method for treating peripheral artery disease (PAD) below the knee, comprising:
inserting into a peripheral artery below the knee at the site of a target lesion of a subject a scaffold made from a material comprising a polymer,
the scaffold having a pattern of interconnected elements, the interconnected elements comprising struts and links, and
performing duplex ultrasound (DUS) and/or angiography after inserting the scaffold into the peripheral artery below the knee to measure binary restenosis at the site of the target lesion,
wherein the binary restenosis is defined as the presence of a hemodynamically significant restenosis of >50% identified through angiography, or peak systolic velocity ratio (PSVR) ≥2.0 as identified by DUS, and
wherein after 1 year after inserting the scaffold into the peripheral artery, the subject exhibits, relative to balloon angioplasty, a non-inferior rate of freedom from both of the following:
(1) a major adverse limb event (MALE), wherein MALE is defined as an above-ankle amputation and/or re-intervention, including thrombosis, at 6 months after inserting the scaffold into the peripheral artery below the knee, and
(2) death within 30 days of inserting the scaffold into the peripheral artery below the knee.

Another aspect of the present disclosure is directed to a method for treating peripheral artery disease (PAD) below the knee, comprising:
inserting into a peripheral artery below the knee at the site of a target lesion of a subject a scaffold made from a material comprising a polymer, the scaffold having a pattern of interconnected elements, the interconnected elements comprising struts and links, the scaffold being configured to, after 1 year after inserting the scaffold into the peripheral artery, reduce, relative to balloon angioplasty, the risk of the following in the subject: (1) a total occlusion of the site of the target lesion, (2) a binary restenosis of the site of the target lesion, and (3) a clinically driven target lesion revascularization (CD-TLR); and
periodically performing duplex ultrasound (DUS) and/or angiography after inserting the scaffold into the peripheral artery below the knee to measure binary restenosis at the site of the target lesion,
wherein the binary restenosis is defined as the presence of the restenosis being a hemodynamically significant restenosis of >50% identified through angiography, or a peak systolic velocity ratio (PSVR) ≥2.0 as identified by DUS.

Another aspect of the present disclosure is directed to a method for treating peripheral artery disease (PAD) below the knee, comprising:
inserting into a peripheral artery below the knee at the site of a target lesion of a target vessel of a subject a scaffold made from a material comprising a polymer, the scaffold having a pattern of interconnected elements, the interconnected elements comprising struts and links, the scaffold being configured to, within 1 year of inserting the scaffold into the peripheral artery, reduce, relative to balloon angioplasty, the risk of all of the following in the patient: (1) a total occlusion of the target vessel, (2) a binary restenosis of the target lesion, and (3) a clinically driven target lesion revascularization (CD-TLR); and
periodically performing duplex ultrasound (DUS) after inserting the scaffold into the peripheral artery below the knee to measure binary restenosis at the site of the target lesion,
wherein the binary restenosis is defined as the presence of the restenosis being a hemodynamically significant restenosis of >50% identified through angiography, or a peak systolic velocity ratio (PSVR) ≥2.0 as identified by DUS.

In any of the described embodiments, the scaffold may be configured to be crimped to a balloon.

In any of the described embodiments, the DUS and/or angiography may be performed at one or more of 30 days (+/−14 days), 3 months (+/−14 days), 6 months (+/−28 days), 1 year (+/−28 days), 2 years (+/−28 days), 3 years (+/−28 days), 4 years (+/−28 days), or 5 years (+/−28 days) after inserting the scaffold into the peripheral artery below the knee. Other time periods and ± intervals around such time period are of course also possible.

In any of the described embodiments, when the DUS indicates the presence of an abnormal reference peak systolic velocity (PSV), the method may further include identifying one or more correlating factors, the correlating factors comprising one or more of:
(1) a focal increase in the absolute peak systolic velocity (PSV) at the site of the target lesion,
(2) a spectral broadening of the waveform at the site of the target lesion,
(3) a post-stenotic turbulence (PST) and/or a change in a waveform shape and/or drop in peak systolic velocity distal to the site of the target lesion, or
(4) a review of one or more B-mode images showing a plaque burden at the site of the target lesion.

In any of the described embodiments, the subject may be selected based on the subject having at least a 70% stenosis at the site of the target lesion prior to insertion of the scaffold.

In any of the described embodiments, the scaffold may be coated with a coating comprised of an active pharmaceutical ingredient and bioresorbable poly (D,L-lactide) (PDLLA).

In any of the described embodiments, the active pharmaceutical ingredient may comprise everolimus, sirolimus, or another Limus drug (or other anti-proliferative agent).

In any of the described embodiments, the polymer may comprise at least one of poly(L-lactide) (“PLLA”), poly(L-lactide-coglycolide) (“PLGA”), polyD-lactide-co-glycolide) or poly (L-lactide-co-D-lactide) (“PLLA-co-PDLA”) with less than 10% D-lactide, or PLLD/PDLA stereo complex.

In any of the described embodiments, the polymer may be bioresorbable (e.g., such that the scaffold is implanted, and left in place, but bioresorbs over time, e.g., within 5 years, 4 years, 3 years, 2 years, or other time period).

In any of the described embodiments, the struts and the links may each have a width and a thickness, and an aspect ratio (AR) of the width to thickness of at least some of the struts or at least some of the links may be from about 0.8 to about 1.4.

In any of the described embodiments, the links may each have a width and a thickness, and an aspect ratio (AR) of the width to thickness of at least some of the links may be from about 0.4 to about 0.9.

In any of the described embodiments, the angiography and/or the DUS may indicate a final residual stenosis of <30% at at least one of 30 days (+/−14 days), 3 months (+/−14 days), 6 months (+/−28 days), 1 year (+/−28 days), 2 years (+/−28 days), 3 years (+/−28 days), 4 years (+/−28 days), or 5 years (+/−28 days) after inserting the scaffold into the peripheral artery below the knee. The risk of a final residual stenosis being greater ≥30% may be reduced, as compared to the standard of care (i.e., an angioplasty treatment).

In any of the described embodiments, the method may further comprise, when the angiography and/or the DUS indicates that there is a binary restenosis, performing an interventional procedure, the interventional procedure being selected from angioplasty, implanting of a second resorbable scaffold or a metallic stent, brachytherapy, atherectomy, thrombectomy, intravascular lithotripsy, remedial surgery, genetic treatment with gene transfer or stem cell infusion.

In any of the described embodiments, if both angiography and DUS are performed, the angiography may take precedence over the DUS for the determination of the presence of binary restenosis.

In any of the described embodiments, the scaffold may exhibit a crush recoverability of at least about 90% after a 50% crushing load.

In any of the described embodiments, the scaffold may exhibit a crush recoverability of at least about 95% after a 50% crushing load.

In any of the described embodiments, the scaffold may exhibit a crush recoverability of at least about 90% after a 75% crushing load.

In any of the described embodiments, the scaffold may exhibit a pinching stiffness of at least about 0.5 N/mm, a radial strength of at least about 0.3 N/mm, and/or a wall thickness of at least about 0.008″, or from about 0.008″ to about 0.014″.

In any of the described embodiments, the peripheral artery is an infrapopliteal artery. Examples of such include, but are not limited to the peroneal and tibial arteries, the tibioperoneal trunk, the anterior tibial artery and the posterior tibial artery.

In any of the described embodiments, the subject may be known to have symptomatic chronic limb threatening ischemia (CLTI) prior to insertion of the scaffold.

In any of the described embodiments, insertion of the scaffold may reduce the risk, relative to balloon angioplasty, of each of (1) a total occlusion of the site of the target lesion, (2) a binary restenosis of the site of the target lesion, and (3) a clinically driven target lesion revascularization (CD-TLR) for at least 1 year after insertion of the scaffold.

In any of the described embodiments, after 1 year after inserting the scaffold into the peripheral artery, the subject may exhibits enhanced freedom (or reduced risk) from the following, relative to balloon angioplasty:
(1) a total occlusion of the site of the target lesion,
(2) binary restenosis of the site of the target lesion, and
(3) a clinically driven target lesion revascularization (CD-TLR).

In any of the described embodiments, insertion of the scaffold may be non-inferior to balloon angioplasty for preventing:
(1) a major adverse limb event (MALE), wherein MALE is defined as an above-ankle amputation and/or re-intervention, including thrombosis, at 6 months after inserting the scaffold into the peripheral artery below the knee, and
(2) death of the subject within 30 days of inserting the scaffold into the peripheral artery below the knee.

In any of the described embodiments, each ring of the scaffold may include struts and crowns, wherein the struts are configured to fold at the crowns when the stent is crimped to the balloon.

In any of the described embodiments, the rings of the scaffold include U crowns, and the links connected to their respective rings may form, with the respective rings, either a W crown or a Y crown.

In any of the described embodiments, the insertion of the scaffold may be effective to reduce the rate of restenosis below the knee to no more than 50% for at least 1 year after insertion of the scaffold into the peripheral artery.

In any of the described embodiments, the insertion of the scaffold may be effective to achieve an improvement of at least 13%, at least 15%, at least 20%, at least 25%, at least 30%, from 13% to 50%, 13% to 45%, 13% to 40% or from 13% to 35%, compared to balloon angioplasty, in treatment of the target lesion, based on one or more, or a composite of the following criteria: freedom from (1) an above ankle amputation, (2) a total occlusion of the site of the target lesion, (3) a binary restenosis of the site of the target lesion, and (4) a clinically driven target lesion revascularization (CD-TLR) for at least 1 year after insertion of the scaffold.

In any of the described embodiments, insertion of the scaffold may be effective to achieve an improvement of at least 11%, at least 15%, at least 20%, or at least 25%, compared to balloon angioplasty, in treatment of the target lesion, for at least 1 year after insertion of the scaffold, based on a binary restenosis of the site of the target lesion.

In any of the described embodiments, insertion of the scaffold may improve CLTI in the subject to a greater extent than a balloon angioplasty standard of care, the improvement being measured by limb salvage and vessel patency, for at least 1 year after insertion of the scaffold.

In any of the described embodiments, insertion of the scaffold may be effective to improve the outcome of treatment in the subject compared to a balloon angioplasty standard of care, for at least 1 year after insertion of the scaffold, wherein the outcome of treatment is measured by limb salvage and vessel patency.

In any of the described embodiments, insertion of the scaffold may be superior to a balloon angioplasty standard of care, as measured by limb salvage and vessel patency, for at least 1 year after insertion of the scaffold.

In any of the described embodiments, insertion of the scaffold may be effective to, relative to balloon angioplasty, reduce the occurrence of target lesion revascularization for at least 1 year after insertion of the scaffold.

In any of the described embodiments, insertion of the scaffold may be effective to reduce a target lesion revascularization rate in the subject compared to a balloon angioplasty standard of care, for at least 1 year after insertion of the scaffold.

In any of the described embodiments, insertion of the scaffold may be noninferior in safety for the subject as a balloon angioplasty standard of care method, wherein safety is defined as freedom from both of the following:
(1) a major adverse limb event (MALE), wherein MALE is defined as an above-ankle amputation and/or re-intervention, including thrombosis, at 6 months after inserting the scaffold into the peripheral artery below the knee, and
(2) death of the subject within 30 days of inserting the scaffold into the peripheral artery below the knee.

In any of the described embodiments, insertion of the scaffold may be effective to reduce the rate of restenosis below the knee to no more than 50% for at least 1 year after insertion of the scaffold into the peripheral artery and may be non-inferior to balloon angioplasty in preventing:
(1) a major adverse limb event (MALE), wherein MALE is defined as an above-ankle amputation and/or re-intervention, including thrombosis, at 6 months after inserting the scaffold into the peripheral artery below the knee, and
(2) death of the subject within 30 days of inserting the scaffold into the peripheral artery below the knee.

In any of the described embodiments, the scaffold may reduce acute vessel recoil to no more than 30%.

In any of the described embodiments, insertion of the scaffold may be effective to achieve an improvement of at least 3%, compared to balloon angioplasty in treatment of the target lesion, based on the following criteria: freedom from a total occlusion of the target vessel for at least 1 year after insertion of the scaffold.

In any of the described embodiments, insertion of the scaffold may be effective to achieve an improvement of at least 3%, 5%, or 7%, compared to balloon angioplasty in treatment of the target lesion, based on the following criteria: freedom from a clinically driven target lesion revascularization (CD-TLR) for at least 1 year after insertion of the scaffold.

In any of the described embodiments, insertion of the scaffold may be effective to achieve an improvement of at least 15%, or at least 20% compared to balloon angioplasty in the treatment of the target lesion, based on binary restenosis, for at least 1 year after insertion of the scaffold.

In any of the described embodiments, insertion of the scaffold may be effective to achieve an improvement of at least 15%, at least 20% or at least 25%, compared to balloon angioplasty, for at least 2 years after insertion of the scaffold, in the treatment of the target lesion, based on a composite of the following criteria: freedom from (1) an above ankle amputation, (2) a total occlusion of the target vessel, (3) a binary restenosis of the target lesion, and (4) a clinically driven target lesion revascularization (CD-TLR).

In any of the described embodiments, insertion of the scaffold may be effective to achieve an improvement of at least 15%, at least 20%, or at least 25%, compared to balloon angioplasty, for at least 2 years after insertion of the scaffold, in the treatment of the target lesion, based on limb salvage and primary patency.

In any of the described embodiments, insertion of the scaffold may be effective to achieve an improvement of at least 15%, or at least 20%, compared to balloon angioplasty, for at least 2 years after insertion of the scaffold, in the treatment of the target lesion, based on binary restenosis.

In any of the described embodiments, insertion of the scaffold may be effective to, relative to balloon angioplasty, reduce the risk of the following for at least 2 years after insertion of the scaffold:
(1) a total occlusion of the target vessel,
(2) a binary restenosis of the target lesion, and
(3) a clinically driven target lesion revascularization (CD-TLR).

In any of the described embodiments, insertion of the scaffold may be non-inferior in terms of safety for the subject, relative to balloon angioplasty, evaluated at 2 years after insertion of the scaffold wherein safety is defined as freedom from both of the following:
(1) a major adverse limb event (MALE), wherein MALE is defined as an above-ankle amputation and/or re-intervention, including thrombosis, at 6 months after inserting the scaffold into the peripheral artery below the knee, and
(2) death of the subject within 30 days of inserting the scaffold into the peripheral artery below the knee.

In any of the described embodiments, the difference in safety, relative to balloon angioplasty, evaluated at 2 years after insertion of the scaffold and based on the p-value, may be insignificant, wherein safety is defined as freedom from both of the following:
(1) a major adverse limb event (MALE), wherein MALE is defined as an above-ankle amputation and/or re-intervention, including thrombosis, at 6 months after inserting the scaffold into the peripheral artery below the knee, and
(2) death of the subject within 30 days of inserting the scaffold into the peripheral artery below the knee.

While various time periods are described for various evaluations (e.g., 1 year, 2 years, etc.), it will be appreciated that any other time periods may be employed, for such evaluation. For example, any of the above noted improvements may extend over a period of greater than 1 year, or greater than 2 years, such as at least 3 years, at least 4 years, at least 5 years, etc. For example, in any of the described embodiments, insertion of the scaffold may be effective to achieve an improvement of at least 5%, at least 10%, at least 15%, at least 20%, or at least 25%, compared to balloon angioplasty, for at least 3 years, at least 4 years, or at least 5 years after insertion of the scaffold, in the treatment of the target lesion, based on limb salvage and primary patency, or other desired performance criteria.

For example, in any of the described embodiments, insertion of the scaffold may be effective to achieve an improvement of at least 5%, at least 10%, at least 15% or at least 20%, compared to balloon angioplasty, for at least 1, 2, 3, 4, or 5 years after insertion of the scaffold, in the treatment of the target lesion, based on a composite of the following criteria: freedom from (1) an above ankle amputation, (2) a total occlusion of the target vessel, (3) a binary restenosis of the target lesion, and (4) a clinically driven target lesion revascularization (CD-TLR).

In another example, insertion of the scaffold may be effective to achieve an improvement of at least 5%, at least 10%, at least 15%, or at least 20%, compared to balloon angioplasty, for at least 1, 2, 3, 4, or 5 years after insertion of the scaffold, in the treatment of the target lesion, based on limb salvage and primary patency.

In another example, insertion of the scaffold may be effective to achieve an improvement of at least 5%, or at least 10%, compared to balloon angioplasty, for at least 1, 2, 3, 4, or 5 years after insertion of the scaffold, in the treatment of the target lesion, based on binary restenosis.

In another example, insertion of the scaffold may be effective to, relative to balloon angioplasty, reduce the risk of the following for at least 1, 2, 3, 4, or 5 years after insertion of the scaffold:
(1) a total occlusion of the target vessel,
(2) a binary restenosis of the target lesion, and
(3) a clinically driven target lesion revascularization (CD-TLR).

In another example, insertion of the scaffold is non-inferior in terms of safety for the subject, relative to balloon angioplasty, evaluated at 1, 2, 3, 4, or 5 years after insertion of the scaffold wherein safety is defined as freedom from both of the following:
(1) a major adverse limb event (MALE), wherein MALE is defined as an above-ankle amputation and/or re-intervention, and
(2) death of the subject within 30 days of inserting the scaffold into the peripheral artery below the knee.

Additional features and advantages of exemplary implementations of the invention will be set forth in the description which follows, and in part will be obvious from the description, or may be learned by the practice of such exemplary implementations. The features and advantages of such implementations may be realized and obtained by means of the instruments and combinations particularly pointed out in the appended claims. These and other features will become more fully apparent from the following description and appended claims or may be learned by the practice of such exemplary implementations as set forth hereinafter.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIG. 1 is a partial planar view of a scaffold pattern according to a first embodiment of a scaffold.

FIG. 2 is a partial perspective view of a scaffold structure.

FIG. 3 is a partial planar view of a scaffold pattern according to a second embodiment of a scaffold.

FIG. 4A is a planar view of a portion of the scaffold pattern of FIG. 3 corresponding to phantom box VA.

FIG. 4B is planar view of a portion of the scaffold pattern of FIG. 1 corresponding to phantom box VB.

FIGS. 5A-5B show a scaffold crown formation in its expanded and crimped states.

FIGS. 5C-5D show a scaffold crown formation in its expanded and crimped states for a scaffold according to the first embodiment.

FIGS. 5E-5F show a scaffold crown formation in its expanded and crimped states for a scaffold according to an alternative embodiment.

FIGS. 6A and 6B are partial planar views of a scaffold pattern according to an alternate embodiment of a scaffold including a first embodiment of a weakened or flexible link element connecting rings.

FIG. 7 a partial planar view of a scaffold pattern according to an alternate ring structure for a scaffold where the ring structure has curved struts extending between crowns.

FIG. 8 shows an exemplary polymeric resorbable scaffold and a schematic of its incorporation into a delivery system for the treatment of peripheral artery disease, for implantation below the knee.

FIG. 9 is a graph comparing the primary efficacy end point for angioplasty versus treatment with a polymeric scaffold, where the primary endpoint was defined as freedom from each of the following events at 1 year: amputation above the ankle of the target limb, occlusion of the target vessel, clinically driven revascularization of the target lesion, and binary restenosis of the target lesion. The Kaplan-Meier curve shows the percentage of patients free from all events from baseline through day 453. The one-sided P value for the primary efficacy end point was calculated on the basis of the Com-Nougue method against a one-sided alpha level of 0.0249.

FIG. 10 is a Kaplan-Meier curve comparing the primary safety endpoint for angioplasty versus treatment with a polymeric scaffold.

FIG. 11 is a Kaplan-Meier curve comparing primary patency and limb salvage (i.e., without binary restenosis) for angioplasty versus treatment with a polymeric scaffold.

FIG. 12 is a Kaplan-Meier curve comparing incidence of binary restenosis for angioplasty versus treatment with a polymeric scaffold.

FIG. 13 is a table showing baseline, procedural, and post-procedural characteristics of observed target lesions for angioplasty versus treatment with a polymeric scaffold.

FIG. 14 is a table showing multiple imputation analysis for angioplasty versus treatment with a polymeric scaffold.

FIG. 15 is a table showing data for primary end points and powered secondary endpoints for angioplasty versus treatment with a polymeric scaffold.

FIG. 16 is a table showing results for procedural end points for angioplasty versus treatment with a polymeric scaffold.

FIGS. 17A-17C show Kaplan-Meier curves for primary safety end point (FIG. 17A), binary restenosis of the target lesion (FIG. 17B), and clinically-driven target lesion revascularization (FIG. 17C) for angioplasty versus treatment with a polymeric scaffold.

FIG. 18 shows Kaplan-Meier curves for primary efficacy endpoint, with cut-off at 393 days for angioplasty versus treatment with a polymeric scaffold.

FIG. 19 shows a partial planar view of an exemplary polymeric scaffold.

FIGS. 20-21 show an exemplary polymeric scaffold comprising a 6-crest, 3-link/ring pattern.

FIG. 22 is a Table of characteristics for four exemplary polymeric scaffolds.

FIG. 23 shows a polymeric scaffold modified for the inclusion of RO markers.

FIGS. 24-25 show an exemplary polymeric scaffold comprising a 7-crest, 3-link/ring pattern.

FIGS. 26-27 show an exemplary polymeric scaffold comprising a 9-crest, 3-link/ring pattern.

FIG. 28 illustrates the theoretical maximum expansion (TME) difference between a smaller TME scaffold (in solid) and a larger TME scaffold (in dotted lines).

FIG. 29 shows a polymeric scaffold modified for the inclusion of RO markers.

FIGS. 30-32 show an exemplary polymeric scaffold comprising a 9-crest, 3-link/ring pattern.

FIG. 33 illustrates an exemplary depiction of scaffold strut dimensions (in inches) for the embodiments depicted in FIGS. 20-21.

FIGS. 34A-34D illustrate characteristics of the exemplary scaffolds depicted in FIGS. 20-21, FIGS. 24-25, FIGS. 26-27, and FIGS. 30-31, respectively.

FIG. 35 is a graph comparing the primary efficacy end point for angioplasty versus treatment with a polymeric scaffold, evaluated at 2 years after insertion of the scaffold, where the primary efficacy endpoint was defined as freedom from each of the following events: amputation above the ankle of the target limb, occlusion of the target vessel, clinically driven revascularization of the target lesion, and binary restenosis of the target lesion.

FIG. 36 is a graph comparing limb salvage and primary patency, evaluated at 2 years after insertion of the scaffold, for angioplasty versus treatment with a polymeric scaffold.

FIG. 37 is a graph comparing freedom from binary restenosis, evaluated at 2 years after insertion of the scaffold, for angioplasty versus treatment with a polymeric scaffold.

FIG. 38 is a graph comparing the second powered secondary endpoint, evaluated at 2 years after insertion of the scaffold, for angioplasty versus treatment with a polymeric scaffold.

FIG. 39 is a graph comparing the primary safety endpoint, evaluated at 2 years after insert of the scaffold, for angioplasty versus treatment with a polymeric scaffold.

FIG. 40 is a graph comparing the primary efficacy end point for angioplasty versus treatment with a polymeric scaffold, evaluated at 3 years after insertion of the scaffold.

FIG. 41 is a graph comparing the primary safety end point for angioplasty versus treatment with a polymeric scaffold, evaluated at 3 years after insertion of the scaffold.

FIG. 42 is a graph comparing freedom from binary restenosis, evaluated at 3 years after insertion of the scaffold, for angioplasty versus treatment with a polymeric scaffold.

FIG. 43 is a graph comparing the clinically-driven target lesion revascularization, evaluated at 3 years after insertion of the scaffold, for angioplasty versus treatment with a polymeric scaffold.

FIG. 44 is a table showing various features for four exemplary polymeric scaffolds.

### DETAILED DESCRIPTION

### I. Introduction

One or more specific embodiments of the present disclosure will be described below. In an effort to provide a concise description of these embodiments, some features of an actual embodiment may be described in the specification. It should be appreciated that in the development of any such actual embodiment, as in any engineering or design project, numerous embodiment-specific decisions will be made to achieve the developers'specific goals, such as compliance with system-related and business-related constraints, which may vary from one embodiment to another. It should further be appreciated that such a development effort might be complex and time consuming, but would nevertheless be a routine undertaking of design, fabrication, and manufacture for those of ordinary skill having the benefit of this disclosure.

All publications and patent applications mentioned in this specification are herein incorporated by reference to the same extent as if each individual publication or patent application was specifically and individually indicated to be incorporated by reference, and as if each said individual publication or patent application was fully set forth, including any figures, herein.

One or more embodiments of the present disclosure may generally relate to the use of bioresorbable polymeric scaffolds below the knee for the treatment of peripheral artery disease, including chronic limb threatening ischemia (CLTI).

The disclosure proceeds as follows. First, definitions of terms that may be used during the course of the subsequent disclosure are explained. Embodiments of processes for forming a deformed polymer tube from a precursor are provided. According to the disclosure, the crush recoverable and balloon expandable scaffold may be cut from a tube formed through a process intended to enhance mechanical properties of the scaffold including fracture toughness. Scaffold patterns according to several embodiments are discussed next. Exemplary scaffold patterns are provided. During this discussion, reference is made to aspects of a scaffold found to play an important role in the stiffness, strength, crimping and deployment of a polymer scaffold, as well as other properties as they relate to crush recoverability of a load-bearing polymer structure. Included herein are aspects of the scaffold that are contrary and, in some cases, surprising and unexpected, particularly when compared to aspects of a comparable, peripheral metal stent having a similar pattern of struts.

Further, in-vivo test results (e.g., clinical trials) of the use of such polymeric scaffolds on below the knee treatment of peripheral artery disease (PAD) are discussed, including examples of embodiments of the invention and explanation of the results observed and problems overcome. In these examples, there may be gained a further appreciation of aspects of the invention—a crush recoverable and balloon-expandable polymer scaffold having desirable radial strength and stiffness properties and capable of being crimped to a diameter suitable for delivery through a peripheral blood vessel via a balloon catheter, such that the scaffold is capable of reducing rates of restenosis to below 30% within a year of its insertion into the peripheral artery. Finally, methods of below the knee treatment of PAD using bioresorbable polymeric scaffolds will be discussed, including treatment of CLTI.

While the present disclosure will describe a particular implementation of below the knee treatment of PAD, it should be understood that the devices, systems, and method described herein may be applicable to other uses. Additionally, elements described in relation to any embodiment depicted and/or described herein may be combinable with elements described in relation to any other embodiment depicted and/or described herein.

### II. Definitions

For purposes of this disclosure, the following terms and definitions apply:

“Inflated diameter” or “expanded diameter” refers to the maximum diameter the scaffold attains when its supporting balloon is inflated to expand the scaffold from its crimped configuration to implant the scaffold within a vessel. The inflated diameter may refer to a post-dilation diameter which is beyond the nominal balloon diameter, e.g., a 6.5 mm semi-compliant PEBAX balloon has about a 7.4 mm post-dilation diameter. The scaffold diameter, after attaining its inflated diameter by balloon pressure, will to some degree decrease in diameter due to recoil effects and/or compressive forces imposed by the wall of the vessel after the balloon is removed. The inflated diameter may be about 1.2 times the average vessel diameter and peripheral below the knee vessel sizes typically range from about 4 to 10 mm for purposes of this disclosure.

The glass transition temperature (referred to herein as “Tg”) is the temperature at which the amorphous domains of a polymer change from a brittle vitreous state to a solid deformable or ductile state at atmospheric pressure. In other words, Tg corresponds to the temperature where the onset of segmental motion in the chains of the polymer occurs. Tg of a given polymer can be dependent on the heating rate and can be influenced by the thermal history of the polymer. Furthermore, the chemical structure of the polymer heavily influences the glass transition by affecting mobility of polymer chains.

“Stress” refers to force per unit area, as in the force acting through a small area within a plane within a subject material. Stress can be divided into components, normal and parallel to the plane, called normal stress and shear stress, respectively. Tensile stress, for example, is a normal component of stress that leads to expansion (increase in length) of the subject material. In addition, compressive stress is a normal component of stress resulting in compaction (decrease in length) of the subject material.

“Strain” refers to the amount of expansion or compression that occurs in a material at a given stress or load. Strain may be expressed as a fraction or percentage of the original length, i.e., the change in length divided by the original length. Strain, therefore, is positive for expansion and negative for compression.

“Modulus” may be defined as the ratio of a component of stress or force per unit area applied to a material divided by the strain along an axis of applied force that result from the applied force. For example, a material has both a tensile and a compressive modulus.

“Toughness” or “fracture toughness” is the amount of energy absorbed prior to fracture, or equivalently, the amount of work required to fracture a material. One measure of toughness is the area under a stress-strain curve from zero strain to the strain at fracture. The stress is proportional to the tensile force on the material and the strain is proportional to its length. The area under the curve then is proportional to the integral of the force over the distance the polymer stretches before breaking. This integral is the work (energy) required to break the sample. The toughness is a measure of the energy a sample can absorb before it breaks. There is a difference between toughness and strength. A material that is strong, but not tough, is said to be brittle. Brittle materials are strong but cannot deform very much before breaking.

As used herein, the terms “axial” and “longitudinal” are used interchangeably and refer to a direction, orientation, or line that is parallel or substantially parallel to the central axis of a stent or the central axis of a tubular construct. The term “circumferential” refers to the direction along a circumference of the stent or tubular construct. The term “radial” refers to a direction, orientation, or line that is perpendicular or substantially perpendicular to the central axis of the stent or the central axis of a tubular construct and is sometimes used to describe a circumferential property, i.e., radial strength.

The term “crush recovery” is used to describe how the scaffold recovers from a pinch or crush load, while the term “crush resistance” is used to describe the force required to cause a permanent deformation of a scaffold. A scaffold or stent that does not possess good crush recovery does not substantially return to its original diameter following removal of a crushing force. As noted earlier, a scaffold or stent having a desired radial force can have an unacceptable crush recovery. And a scaffold or stent having a desired crush recovery can have an unacceptable radial force.

### III. Exemplary Polymer Scaffolds

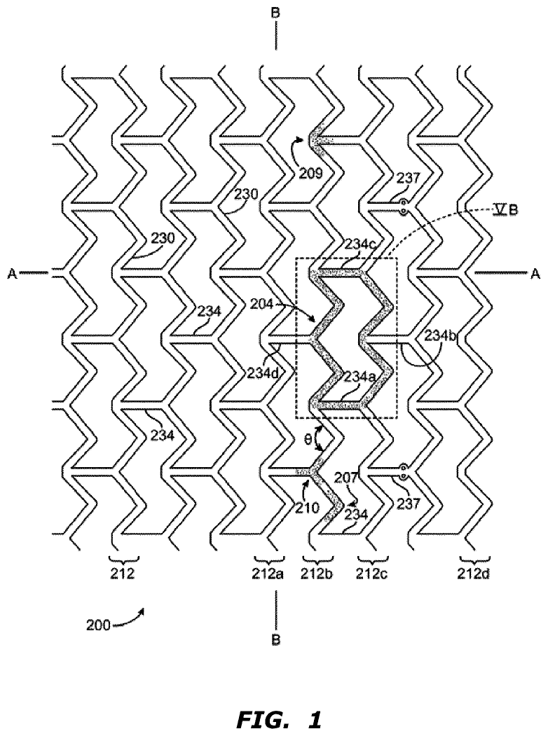

An exemplary polymer scaffold, illustrated in FIG. 1, is formed from a poly(L-lactide) (“PLLA”) tube. The process for forming this PLLA tube may be the process described in U.S. Publication No. 2011/0066222, which is herein incorporated by reference in its entirety. Reference is made to a precursor that is “deformed” in order to produce a tube having the desired scaffold diameter, thickness, and material properties as set forth below. Before the tube is deformed or, in some embodiments, expanded to produce the desired properties in the starting tube for the scaffold, the precursor is formed. The precursor may be formed by an extrusion process that starts with raw PLLA resin material heated above the melt temperature of the polymer, which is then extruded through a die. Then, in one example, an expansion process for forming an expanded PLLA tube includes heating a PLLA precursor above the PLLA glass transition temperature (e.g., 60-70° C.) but below the melt temperature (e.g., 165-175° C.), e.g., around 110-120° C.

A precursor tube is deformed in radial and axial directions by a blow molding process wherein deformation occurs progressively at a predetermined longitudinal speed along the longitudinal axis of the tube. As explained below, the deformation improves the mechanical properties of the tube before it is formed into the scaffold of FIG. 1. The tube deformation process is intended to orient polymer chains in radial and/or biaxial directions. The orientation or deformation causing re-alignment is performed according to a precise selection of processing parameters—e.g. pressure, heat (i.e., temperature), deformation rate—to affect material crystallinity and type of crystalline formation during the deformation process.

In an alternative embodiment the tube may be made of poly(L-lactide-co-glycolide), poly(D-lactide-co-glycolide) (“PLGA”), polycaprolactone (“PCL”), any semi-crystalline copolymers combining any of these monomers, or any blends of these polymers. Material choices for the scaffold should take into consideration the complex loading environment associated with many peripheral vessel locations, particularly those located in the contemplated below the knee limb locations.

Peripheral blood vessels provide a dynamic environment for vascular implants, as various forces may crush, twist, extend, or shorten the device simultaneously. The force application may vary between point load to distributed load, or a combination thereof, and also as a function of time. Results have shown that bioresorbable scaffolds made from highly crystalline PLLA can provide crush recovery without causing a permanent and constant outward radial force on the vessel, which characteristic is advantageous and desirable. The permanent and constant outward radial force may be the cause of late clinical issues with nitinol self-expandable stents, as discussed above. However, a remaining challenge with bioresorbable scaffolds is to make them optimally fracture resistant as a function of time—that is, to improve their fatigue life or survivability under a variety of dynamic loading environments. Improved fracture toughness for a scaffold, particularly in a peripherally implanted scaffold can be important.

Such peripheral arteries, into which the polymeric scaffolds of this disclosure are designed for implant, include those below the knee, such as the infrapopliteal arteries (i.e., below the popliteal), such as the peroneal and tibial arteries (e.g., tibioperoneal trunk, peroneal, anterior tibial and posterior tibial arteries). In an embodiment, placement in any arteries above the knee (e.g., femoral, coronary, or elsewhere) is specifically excluded.

The fracture resistance of a vascular scaffold depends not only on the design and the material, but also the manufacturing process and deployment parameters. Therefore, it is particularly important to have a process, design, and delivery system that allows the scaffold to be uniformly expanded and deployed. As a consequence of non-uniform deployment, the various struts and crowns of a scaffold will potentially be exposed to very different forces and motions, which has a deleterious effect on the fatigue life.

A dimensionless number useful for characterizing a material's fracture toughness is called a Deborah number (ratio of intrinsic material damping time constant and time constant of external applied force). The higher the Deborah number, the greater is the expected potential of an implant to fracture under a transient load or fatigue load of a given amplitude.

Toughening domains can be introduced into an implant design in several ways: a) backbone alteration to include low Tg blocks, e.g. block copolymers, b) polymer blends, and c) introducing light crosslinks into the backbone.

Fracture toughness of a homopolymer such as PLLA can also be improved by controlling the microstructure of the final implant. Variables that affect the fracture toughness include percent crystallinity, size and/or distribution of crystallites, spatial distribution, and gradient and shape of the crystalline domains. A combination of these micro-structural controls, in combination with a macroscopic design (e.g., scaffold pattern, crimping process, etc.), may improve fracture toughness without significant adverse effects on other scaffold material properties (e.g., radial and/or pinching stiffness).

An alternative to providing elastomeric properties is the use of a multilayered structure having “soft” and “hard” layers, where the soft layer/layers would be made from a relatively low Tg material and the hard layers would have a relatively high Tg material. In a similar way, high and low Tg domains can generate typical rubber-toughened morphologies through the use of block copolymers or polymer blends. The Tg of a given domain/block could be generated from a given monomer or through the use of several monomers in a random co-polymer. Typical low Tg materials can be made from caprolactone, lactone derivatives, carbonate, butylsuccinate, trimethylene carbonate, dioxanone, or other suitable monomers compatible with the disclosure. Other appropriate low Tg materials include materials that clear the kidneys through dissolution rather than degradation. Such materials may include polyethylene glycol (PEG), polyvinylpyrrolidone (PVP), polyvinylalochol (PVA), or other polymers compatible with the disclosure.

Alternative ways to improve the fatigue properties of the scaffold are through introduction of axial flexibility and the use of pre-designed fracture points, in particular in the connector links. The fracture points could function as precursors of actual fractures, e.g., crazes and cracks or small dimension of fracture distributed in the implant. A distribution or pattern of cracks or crazes may dictate or inform one of an expected toughness of the scaffold when subjected to a particular loading, e.g., torsion, radial force, tensile etc. Although it is understood that, due to the generally highly non-linear relationship between crack formation and a coupled loading environment, that is, simultaneously applied and time varying bending, torsion and axial loading, such predictive methods may not be applicable to all situations.

Alternative ways to improve the fatigue properties are through introduction of axial flexibility and the use of pre-designed fracture points, in particular, fracture points in or near connector links as discussed in U.S. Publication No. 2022/0226133, herein incorporated by reference in its entirety.

The degree of radial expansion that the polymer tube undergoes characterize the degree of induced circumferential molecular and crystal orientation as well as strength in a circumferential direction. The degree of radial expansion is quantified by a radial expansion (“RE”) ratio, defined as RE Ratio =(Inside Diameter of Expanded Tube)/(Original Inside Diameter of the tube). The RE ratio can also be expressed as a percentage. The degree of axial extension that the polymer tube undergoes can partially characterize induced axial molecular or crystal orientation as well as strength in an axial direction. The degree of axial extension is quantified by an axial extension (“AE”) ratio, defined as AE Ratio=(Length of Extended Tube)/(Original Length of the Tube). The AE ratio can also be expressed as a percentage. In a preferred embodiment the RE is 350% to 450%, e.g., about 400% and the AE is 40-50%.

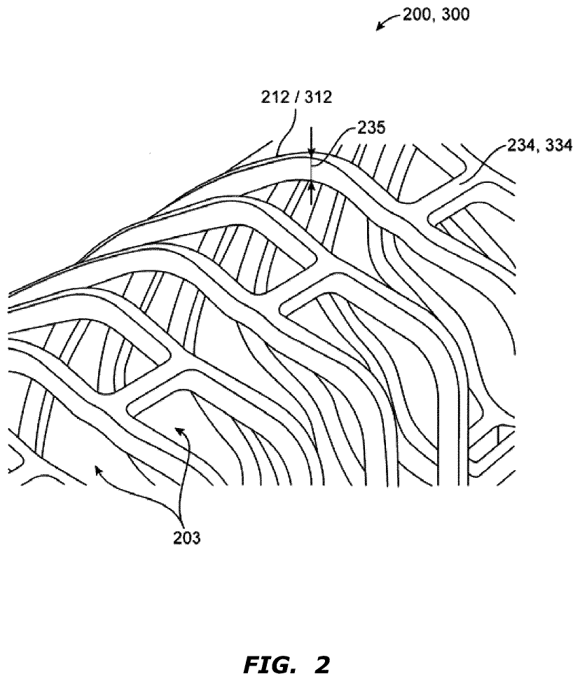

The strengthened and toughened cylindrical, polymer tube is formed into a scaffold structure, in one embodiment a structure having a plurality of struts 230 and links 234 forming a pattern 200 as shown in FIG. 1 (pattern 200 is illustrated in a planar or flattened view), which is about the pattern for the scaffold before crimping and after the scaffold is plastically, or irreversibly deformed from its crimped state to its deployed state within a vessel by balloon expansion. The pattern 200 of FIG. 1, therefore, represents a tubular scaffold structure (as partially shown in three-dimensional space in FIG. 2), so that an axis A-A is parallel to the central or longitudinal axis of the scaffold. FIG. 2 shows the scaffold in a state prior to crimping or after deployment. As can be seen from FIG. 2, the scaffold comprises an open framework of struts and links that define a generally tubular body. The cylindrical, deformed tube of FIG. 2 may be formed into this open framework of struts and links described in FIGS. 1-2 by a laser cutting device, preferably, a pico-second green light laser that uses helium gas as a coolant during cutting.

Details of a suitable laser process can be found in U.S. Pat. No. 8,679,394, herein incorporated by reference in its entirety. The helium gas is beneficial to avoid melting or altering properties of the scaffold structure adjacent the laser's cutting path. Exemplary laser machining parameters are provided in Table 1 in U.S. Publication No. 2022/0226133, herein incorporated by reference in its entirety.

Referring to FIG. 1, the pattern 200 includes longitudinally spaced rings 212 formed by struts 230. A ring 212 is connected to an adjacent ring by several links 234, each of which extends parallel to axis A-A. In this first embodiment of a scaffold pattern (pattern 200) four links 234 connect the interior ring 212, which refers to a ring having a ring to its left and right in FIG. 1, to each of the two adjacent rings. Thus, ring 212b is connected by four links 234 to ring 212c and four links 234 to ring 212a. Ring 212d is an end ring connected to only the ring to its left in FIG. 1.

A ring 212 is formed by struts 230 connected at crowns 207, 209 and 210. A link 234 is joined with struts 230 at a crown 209 (W-crown) and at a crown 210 (Y-crown). A crown 207 (free-crown) does not have a link 234 connected to it. Preferably the struts 230 that extend from a crown 207, 209 and 230 at a constant angle from the crown center, i.e., the rings 212 are approximately zig-zag in shape, as opposed to sinusoidal for pattern 200, although in other embodiments a ring having curved struts is contemplated. As such, in this embodiment a ring 212 height, which is the longitudinal distance between adjacent crowns 207 and 209/210 may be derived from the lengths of the two struts 230 connecting at the crown and a crown angle 0. In some embodiments the angle 0 at different crowns will vary, depending on whether a link 234 is connected to a free or unconnected crown, W-crown or Y-crown.

The zig-zag variation of the rings 212 occurs primarily about the circumference of the scaffold (i.e., along direction B-B in FIG. 1). The struts 212 centroidal axes lie primarily at about the same radial distance from the scaffold's longitudinal axis. Ideally, substantially all relative movement among struts forming rings also occurs axially, but not radially, during crimping and deployment. Although, as explained in greater detail, below, polymer scaffolds often do not deform in this manner due to misalignments and/or uneven radial loads being applied.

The rings 212 are capable of being collapsed to a smaller diameter during crimping and expanded to a larger diameter during deployment in a vessel. According to one aspect of the disclosure, the pre-crimp diameter (e.g., the diameter of the axially and radially expanded tube from which the scaffold is cut) is always greater than a maximum expanded scaffold diameter that the delivery balloon is capable of producing when inflated. According to one embodiment, a pre-crimp diameter is greater than the scaffold expanded diameter, even when the delivery balloon is hyper-inflated, or inflated beyond its maximum use diameter for the balloon-catheter.

Pattern 200 includes four links 237 (two at each end, only one end shown in FIG. 1) having structure formed to receive a radiopaque material in each of a pair of transversely-spaced holes formed by the link 237. These links are constructed in such a manner as to avoid interfering with the folding of struts over the link during crimping, which, as explained in greater detail below, provides for a scaffold capable of being crimped to a diameter of about at most Dₘᵢₙ or for a scaffold that when crimped has virtually no space available for a radiopaque marker-holding structure.

A second embodiment of a scaffold structure has the pattern 300 illustrated in FIG. 3. Like the pattern 200, the pattern 300 includes longitudinally-spaced rings 312 formed by struts 330. A ring 312 is connected to an adjacent ring by several links 334, each of which extends parallel to axis A-A. The description of the structure associated with rings 212, struts 230, links 234, and crowns 207, 209, 210 in connection with FIG. 1, above, also applies to the respective rings 312, struts 330, links 334 and crowns 307, 309 and 310 of the second embodiment, except that in the second embodiment there are only three struts 334 connecting each adjacent pair of rings, rather than four. Thus, in the second embodiment the ring 312b is connected to the ring 312c by only three links 234 and to the ring 312a by only three links 334. A link formed to receive a radiopaque marker, similar to link 237, may be included between 312c and ring 312d.

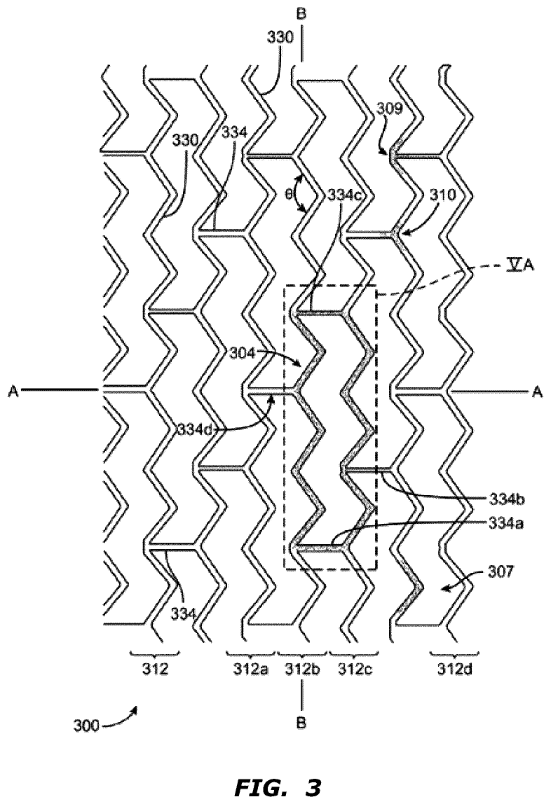

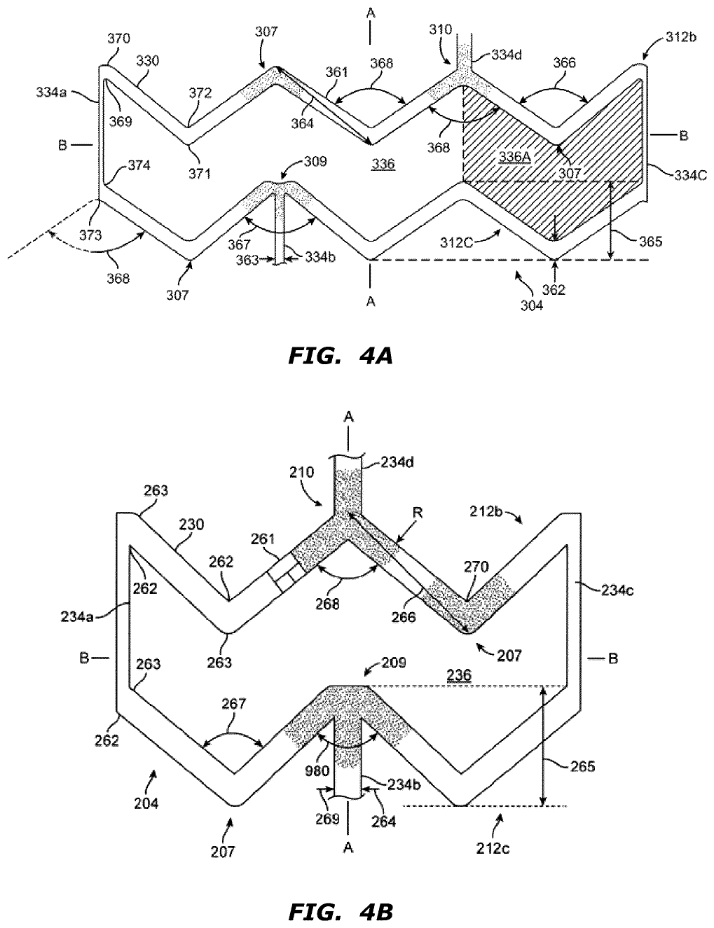

FIGS. 4A and 4B depict aspects of the repeating pattern of closed cell elements associated with each of the patterns 300 and 200, respectively. FIG. 4A shows the portion of pattern 300 bounded by the phantom box VA and FIG. 4B shows the portion of pattern 200 bounded by the phantom box VB. Therein are shown cell 304 and cell 204, respectively. In FIGS. 4A, 4B the vertical axis reference is indicated by the axis B-B and the longitudinal axis A-A. There are four cells 204 formed by each pair of rings 212 in pattern 200, e.g., four cells 204 are formed by rings 212b and 212c and the links 234 connecting this ring pair, another four cells 204 are formed by rings 212a and 212b and the links connecting this ring pair, etc. In contrast, there are three cells 304 formed by a ring pair and their connecting links in pattern 300.

Referring to FIG. 4A, the space 336 and 336a of cell 304 is bounded by the longitudinally spaced rings 312b and 312c portions shown, and the circumferentially spaced and parallel links 334a and 334c connecting rings 312b and 312c. Links 334b and 334d connect the cell 304 to the right and left adjacent ring in FIG. 2, respectively. Link 334b connects to cell 304 at a W-crown 309. Link 334d connects to cell 304 at a Y-crown 310. A “Y-crown” refers to a crown where the angle extending between a strut 330 and the link 334 at the crown 310 is an obtuse angle (greater than 90 degrees). A “W-crown” refers to a crown where the angle extending between a strut 330 and the link 334 at the crown 309 is an acute angle (less than 90 degrees). The same definitions for Y-crown and W-crown also apply to the cell 204. There are eight connected or free crowns 307 for cell 304, which may be understood as eight crowns devoid of a link 334 connected at the crown. There are one or three free crowns between a Y-crown and W-crown for the cell 304.

Additional aspects of the cell 304 of FIG. 4A include angles for the respective crowns 307, 309 and 310 Those angles, which are in general not equal to each other, are identified in FIG. 4A as angles 366, 367 and 368, respectively associated with crowns 307, 309 and 310. For the scaffold having the pattern 300 the struts 330 have strut widths 361 and strut lengths 364, the crowns 307, 309, 310 have crown widths 362, and the links 334 have link widths 363. Each of the rings 312 has a ring height 365. The radii at the crowns are, in general, not equal to each other. The radii of the crowns are identified in FIG. 4A as radii 369, 370, 371, 372, 373 and 374.

Cell 304 may be thought of as a W-V closed cell element. The “Y” portion refers to the shaded area 336a that resembles the letter “V” in FIG. 4A. The remaining unshaded portion 336, i.e., the “W” portion, resembles the letter “W”.

Referring to FIG. 4B, the space 236 of cell 204 is bounded by the portions of longitudinally spaced rings 212b and 212c as shown, and the circumferentially spaced and parallel links 234a and 234c connecting these rings. Links 234b and 234d connect the cell 204 to the right and left adjacent rings in FIG. 1, respectively. Link 234b connects to cell 236 at a W-crown 209. Link 234d connects to cell 236 at a Y-crown 210. There are four crowns 207 for cell 204, which may be understood as four crowns devoid of a link 234 connected at the crown. There is only one free crown between each Y-crown and W-crown for the cell 204.

Additional aspects of the cell 204 of FIG. 4B include angles for the respective crowns 207, 209 and 210. Those angles, which are in general not equal to each other, are identified in FIG. 4B as angles 267, 269 and 268, respectively associated with crowns 207, 209 and 210. For the scaffold having the pattern 200, the struts 230 have strut widths 261 and strut lengths 266, the crowns 207, 209, 210 have crown widths 270, and the links 234 have link widths 264. Each of the rings 212 has a ring height 265. The radii at the crowns are, in general, not equal to each other. The radii of the crowns are identified in FIG. 4B as inner radii 262 and outer radii 263.

Cell 204 in FIG. 4B may be thought of as a W closed cell element. The space 236 bounded by the cell 204 resembles the letter “W”.

Comparing FIG. 4A to FIG. 4B, one can appreciate that the W cell 204 is symmetric about the axes B-B and A-A, whereas the W-V cell 304 is asymmetric about both of these axes. The W cell 204 is characterized as having no more than one crown 207 between links 234. Thus, a Y-crown crown or W-crown is always between each crown 207 for each closed cell of pattern 200. In this sense, pattern 200 may be understood as having repeating closed cell patterns, each having no more than one crown that is not supported by a link 234. In contrast, the W-V cell 304 has three unsupported crowns 307 between a W-crown and a Y-crown. As can be appreciated from FIG. 4A, there are three unsupported crowns 307 to the left of link 334d and three unsupported crowns 307 to the right of link 334b.

Additional aspects of the scaffold pattern(s) according to the present disclosure can be found in U.S. Publication No. 2022/0226133, which is herein incorporated by reference in its entirety.

The mechanical behavior of a scaffold having a pattern 200 verses 300 differs in various ways. These differences, along with others to be discussed later, have been observed in comparisons between the scaffolds. A summary of mechanical differences is included FIGS. 6A-6B of U.S. Publication No. 2022/0226133. In-vivo testing differences also exist. In certain regards, these tests demonstrated mechanical aspects of scaffolds according to the disclosure that were both unexpected and contrary to conventional wisdom, such as when the conventional wisdom originated from state-of-the-art metallic stents, or coronary scaffolds. For a particular design choice, whether driven by a clinical, production yield, and/or delivery profile requirement, therefore, the following characteristics should be kept in mind.

In general, a polymer scaffold that is crush-recoverable, possesses a desired radial stiffness and strength, fracture toughness, and the capability of being crimped down to a target delivery diameter, e.g., at least about Dₘᵢₙ, balances the three competing design attributes of radial strength/stiffness verses toughness, in-vivo performance verses compactness for delivery to a vessel site, and crush recovery verses radial strength/stiffness.

In-vivo performance verses compactness for delivery to the vessel site refers to the ability to crimp the scaffold down to the delivery diameter. The ring struts 230 connecting crowns to form the W-cell 204 are more restrained from rotating about an axis tangent to the abluminal surface (axis A-A). In the case of the W-V cell, the V portion, the crown may tend to twist about the axis A-A under particular configurations due to the reduced number of connecting links 336. The ring portions can in effect “flip”, which means rotate or deflect out-of-plane as a result of buckling (please note: “out-of-plane” refers to deflections outside of the arcuate, cylindrical-like surface of the scaffold; referring to FIG. 4A, “out-of-plane” means a strut that deflects normal to the surface of this figure). When there is a link 234 at each of a crown or valley, as in FIG. 4B, any tendency for the crown to buckle or flip is reduced because the ring struts are more restrained by the link 236. Essentially, the link serves to balance the load across a ring more evenly.

The “flipping” phenomenon for a scaffold constructed according to pattern 300 has been observed during crimping, as explained and illustrated in greater detail in U.S. Pat. No. 8,539,663, herein incorporated by reference in its entirety. The W-V cell 304 is devoid of a nearby link 334 at a crown 307 to restrain excessive twisting of the adjacent crown or valley. In essence, when there are two crowns 307 between a link 334, the restraint preventing flipping or buckling of the V portion of the ring depends on the buckling strength of the individual ring strut 330 (i.e., the strength and stiffness of the polymer strut in torsion). When there is a link 234 connected to each adjacent crown/valley (FIG. 4B), however, out of plane deflections at the crown 207 are restrained more, due to the bending stiffness added by the connected link 234, which restrains twisting at the adjacent crown 207.

A scaffold according to pattern 200 is correspondingly stiffer than a similarly constructed scaffold according to pattern 300. The scaffold according to pattern 200 will be stiffer both axially and in longitudinal bending, since there are more links 236 used. Increased stiffness may not, however, be desirable. Greater stiffness can produce greater crack formation over a less stiff scaffold. For example, the stiffness added by the additional links can induce more stress on rings interconnected by the additional links 234, especially when the scaffold is subjected to a combined bending (rings moving relative to each other) and radial compression and/or pinching (crushing). The presence of the link 234 introduces an additional load path into a ring, in addition to making the ring more stiff.

In-vivo requirements can favor a scaffold according to pattern 200, but a scaffold according to pattern 300 may be more easily crimped down to the delivery diameter. Other factors also affect the ability to crimp a scaffold. According to the disclosure, it was found that crown angles less than about 115 degrees for the pre-crimp scaffold can produce less fracture and related deployment problems (e.g., uneven folding/unfolding of ring struts) than a scaffold with higher crown angles (relative to the inflated diameter, in one case 6.5 mm). The scaffold is crimped to a balloon that can be inflated up to about 7.4 mm. Thus, when the balloon is hyper-inflated, the scaffold attains up to about a 7 mm inflated diameter. For a balloon catheter-scaffold assembly according to the disclosure, the largest inflated diameter for the balloon is less than or equal to the scaffold diameter before crimping. As mentioned above, it is preferred that the maximum inflated diameter for the scaffold is less than the scaffold diameter before crimping.

During the course of designing a crush recoverable polymer scaffold having a desired crimped profile, it was found that when forming the scaffold at the 8 mm diameter, it was difficult to crimp the scaffold to a desired crimped profile (e.g., to crimp the scaffold from the 8 mm diameter to about 2 mm profile) for two reasons. First, by imposing the 350-400% diameter reduction requirement, the polymer material was more susceptible to crack formation and propagation, due to strain levels experienced by the scaffold when subjected to this extensive diameter reduction. This concern was addressed by adjusting stiffness, e.g., reducing the strut angle, wall thickness and/or number of crowns. Additionally, the process steps used to form the polymer tube was found to help improve the scaffold's resistance to crack formation and propagation, as explained earlier.

Second, even when the scaffold dimensions were adjusted to limit crack formation, there was the problem of limited space for the scaffold within the crimped profile. Due to the mass of material associated with the crimped scaffold, the available space for compression of the rings to the desired crimped profile was not achievable without creating unacceptable yield stresses or fracture. Thus, even when a 350-400% diameter reduction was achievable without crack or deployment problems, the scaffold pattern would not allow further reduction without exceeding the range of articulation that the scaffold design would allow.

According to another aspect of the disclosure, there are modified crown designs for a scaffold intended to improve the fracture toughness and/or reduce the delivery diameter of the scaffold. It was discovered that a design change to an existing scaffold pattern that would overcome a limitation on reduced profile, and which could be implemented using a relatively brittle polymer like PLLA or PLGA, was a significant reduction in the size of the inner radius of the crown or valley bridging the struts that form the crown/valley.

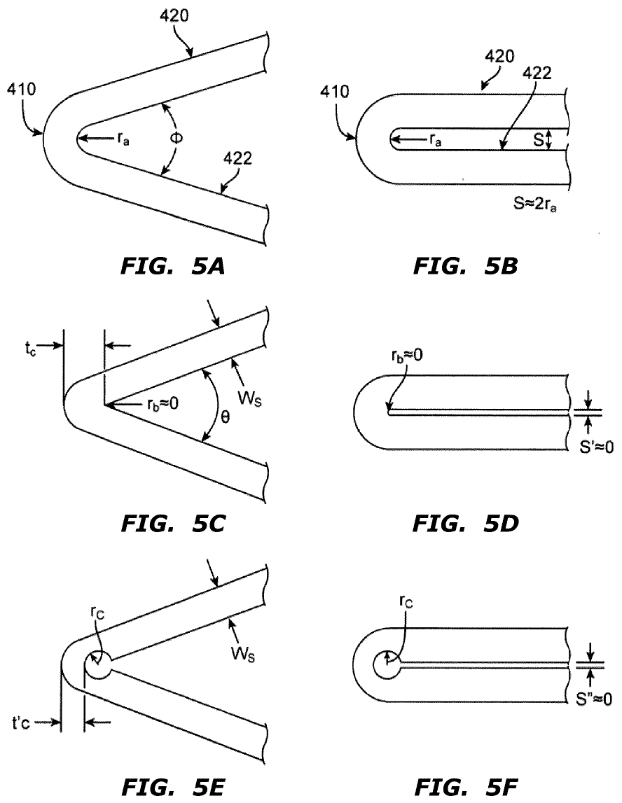

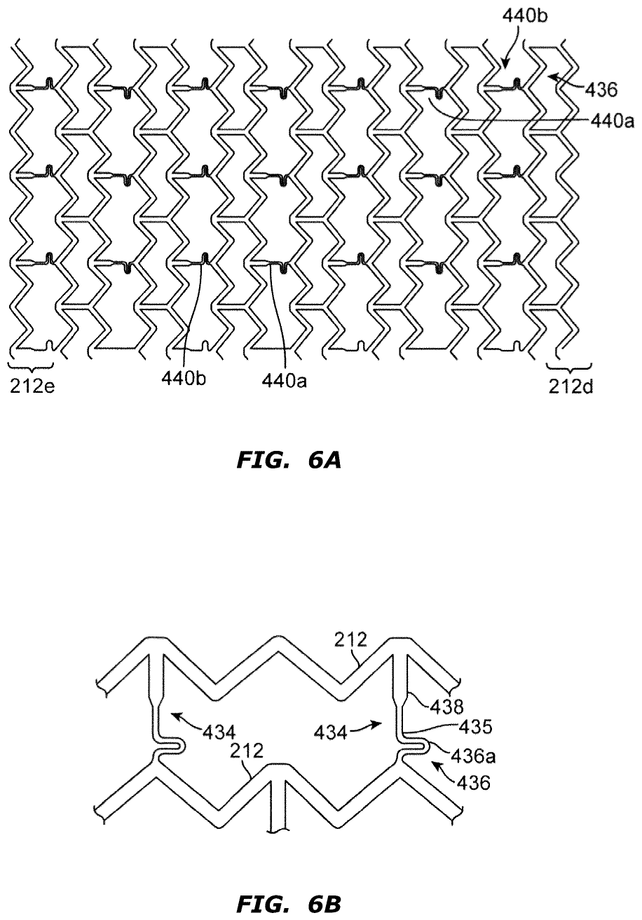

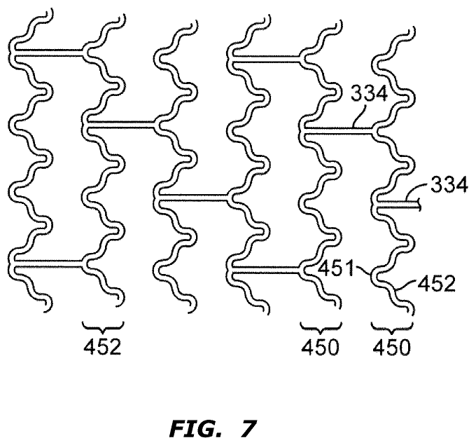

FIGS. 5A and 5B illustrate a pair of struts 420, 422 near a crown 410. In the pre-crimp state, the struts 420, 422 are separated by the crown angle f and the crown is formed with an inner radius rₐ This is a typical design for a crown. The inner radius is selected to avoid stress concentrations at the crown. When there is a dramatic change in geometry at a hinge point, such as a crown, there is a greater likelihood cracks will form or or yielding will occur at the hinge point (thereby affecting radial strength) since the moment of inertia in bending across the crown is discontinuous.

In the case of a metal stent, the angle f before crimping is less than the angle when the stent is deployed. By forming the stent with the reduced diameter, the stent may be more easily crimped to a small profile. Due to the presence of the inner radius, the angle f is capable of being exceeded at deployment without loss of radial stiffness. If this radius is too small, however, and the strut angle at deployment exceeds f, there is a greater chance of yielding or other problems due to stress concentrations at the inner radius. Due to the ductility and resiliency of metal, stents made from metal may also be crimped down further than shown in FIG. 5B. The struts 420, 422 may touch each other, i.e., S is less than 2×rₐ, and yet the stent can still recover and maintain its radial stiffness despite the over-crimped condition.

For a polymer scaffold, however, it has been found that the distance S (FIG. 5B) should not generally be smaller than allowed for the radius rₐ—i.e., S should be greater than or equal to 2 rₐ. For a polymer scaffold, if the struts 420, 422 are brought closer to each other, i.e., S becomes less than 2×rₐ, the brittleness of the material can likely result in fracture problems when the scaffold is deployed. The scaffold may not, therefore, be able to maintain its radial stiffness if crimped beyond the allowable distance for the radius. Fractures at crowns may occur when the distance S in FIG. 5B is less than 2×rₐ. When this occurs, there is significant material failure in a W crown, free crown, and Y crown. Scanning electron microscopy (SEM)—images of such a scenario can be found in U.S. Publication No. 2022/0226133, herein incorporated by reference in its entirety.

With the objective of decreasing the distance S between struts 420, 422 (FIG. 5B), applicant decided to reduce the radius rₐ to as small as possible, despite suggestions in the art to the contrary. It was discovered, to applicant's surprise, that the scaffold was able to recover from the crimped condition to the expanded condition without significant, noticeable, reoccurring, or prohibitive loss in radial strength.

FIGS. 5C and 5D illustrate embodiments of a crown formation corresponding to these unexpected results. An example of a W cell having a reduced radii type of crown formation just described is illustrated in FIGS. 5B and 6B. The radius r_{b}, is about 0.00025 inches, which corresponds to the smallest radius that could be formed by the laser. The 0.00025 inch radius is not contemplated as a target radius or limit on the radius size, although it has produced the desired result for this embodiment. Rather, it is contemplated that the radius may be as close to zero as possible to achieve a reduced profile size. The radius, therefore, in the embodiments can be about 0.00025 (depending on the cutting tool), greater than this radius, or less than this radius to practice the invention in accordance with the disclosure, as will be appreciated by one of ordinary skill in the art. For instance, it is contemplated that the radii may be selected to reduce the crimped size as desired.

An inner radius at about zero, for purposes of the disclosure, means the minimum radius possible for the tool that forms the crown structure. An inner radius in accordance with some embodiments means the radius that allows the distance S to reduce to about zero—i.e., struts are adjacent and/or touch each other as shown in FIG. 5D (S′ is about, or zero).

Without wishing to be tied to a particular theory for how the scaffold according to the invention is capable of being reduced to the theoretical minimum diameter and then expanded without loss of strength, it is believed that the selection of starting diameter being greater than the inflated diameter played a role in the favorable outcome. In contrast to the previous example, where a metal stent is formed from a diameter less than its inflated diameter, which smaller diameter may be selected to facilitate a smaller crimped profile, a polymer scaffold according to preferred embodiments is formed from a starting diameter greater than the maximum inflated diameter for the balloon catheter scaffold assembly (a larger starting diameter may be preferred to reduce acute recoil, as explained below, and/or to enhance radial strength characteristics in the deployed state, as explained earlier in the tube processing steps for the polymer tube). As such, the strut angle pre-crimp is preferably greater than the maximum crown/strut angle when the scaffold is deployed. Stated differently, the crown angle in FIG. 5C (pre-crimp angle) is never exceeded when the balloon expands the scaffold from the crimped to deployed state. This characteristic of the crush recoverable polymer scaffold, i.e., pre-crimp crown angle greater than the deployed crown angle, is believed to provide clues as to how the polymer scaffold was able to retain radial strength when a minimum inner radius was used for the crown formation, contrary to the prior art. Compression, but not expansion, of the scaffold when loaded by the vessel, it is believed, will not induce further weakening, despite the presence of voids. When the crown experiences only a compressive deformation relative to its pre-crimp shape (FIG. 5C), the potentially weakened area near the inner radius is subjected to only compressive stresses, which do not tend to tear the crown apart, i.e., induce crack propagation.

Crimping of the scaffold, as detailed in U.S. Pat. No. 8,539,663, includes heating the polymer material to a temperature less than, but near to, the glass transition temperature of the polymer. In one embodiment, the temperature of the scaffold during crimping is raised to about 5 to 10 degrees below the glass transition temperature for PLLA. When crimped to the final, crimped diameter, the crimping jaws are held at the final crimp diameter for a final dwell period. This method of crimping a polymer scaffold having crush recovery is advantageous for reducing recoil when the crimp jaws are released. Another, unexpected, outcome relating to the reduced inner radius aspect of the disclosure was found—it was discovered that during the dwell period, the polymer scaffold crimped profile could be reduced to a profile less than the theoretical minimum profile.

For one exemplary polymeric scaffold according to the present disclosure, the value for Dₘᵢₙ is 0.1048″ or 2.662 mm. When crimping this scaffold according to the crimping procedure summarized above and described in U.S. Pat. No. 8,539,663, it was found that the scaffold could be reduced to a crimped profile of 0.079″ or 2.0066 mm. Hence, the crimped profile was less than Dₘᵢₙ for this scaffold. With this profile, a protective sheath of 0.085″ OD could be placed over the scaffold. When a drug coating was disposed over the scaffold, the profile of the scaffold with sheath was 0.092″. For this scaffold, the range of radial strength was 0.45-0.65 N/mm, range of radial stiffness was 1.00-1.20 N/mm and the crush recoverability was about 90% (after a 50% crush).

It is believed that a reduced profile less than Dₘᵢₙ was achieved due to a compression of the material during the dwell period. Essentially, the pressure imposed by the crimping jaws during the dwell period at the raised temperature caused the struts forming the ring to be squeezed together to further reduced the crimped scaffold profile. According to these embodiments, the crimped scaffold having a profile less than its theoretical minimum profile was successfully deployed and tested in vivo. This scaffold possessed the desired radial stiffness properties, in addition to the desired crush recovery of above about 90% following a 50% reduction in diameter.

In another aspect of this disclosure, the strut and crown formation for a crush recoverable polymer scaffold is formed to take the shape depicted in FIG. 5E, for purposes of achieving a crimped profile less than the crimped profile for the scaffold having the crown formation shown in FIG. 5A. According to these embodiments, the crown is formed with a radius r_{c}, as shown. When this scaffold is crimped, the struts may be brought close together so that the distance separating them is near zero (S″ is about, or zero, similar to the separation distance in FIG. 5D). In contrast to the embodiments of FIG. 5C, the radius r_{c}, is made some finite or larger radii than by forming a hole or enlarged area between the ends of the struts and crown. The thickness at the crown, t_{c}′ forming the inner radius along its inner surface may be less than the strut width (in the example of FIG. 5C, the crown thickness may be larger than the strut width). This can allow a larger inner radius to be used at the crown without increasing the crimped profile.

In these embodiments, a scaffold having the crown formation depicted in FIGS. 5E-5F is referred to as a “key-hole” crown formation. The name will be understood without further clarification by reference to FIG. 5F, which shows a key-hole slot or opening formed by the inner wall surfaces. In the crimped profile, the struts near the crown may be brought closer together while a hole or opening having radius r_{c} is more or less maintained at the crown. The distance S″ is less than twice the radius r_{c} for the “key-hole”crown formation.

Examples of scaffolds embodying patterns 300 and 200 are provided in FIGS. 6A-6B (referred to as the V2 embodiment, which has a 0.008 inch wall thickness, V23 embodiments having 0.008 and 0.014 inch wall thickness and the V59 embodiment, which has a 0.011 inch wall thickness) of U.S. Publication No. 2022/0226133, herein incorporated by reference in its entirety. Specific values for the various cell attributes of FIGS. 5A-5B are provided in those figures. One scaffold (pattern 200) having a pre-crimp diameter of 8 mm is capable of being crimped to a non-compliant balloon wherein the crimped profile is about 2 mm. The inflated diameter is about 6.5 mm in this example. Other scaffolds having pre-crimp diameters of 7 mm and 9 mm, respectively, are expanded to about 6.5 mm by a non-compliant balloon. Those scaffolds are capable of being crimped to diameters of about 0.092 inches (2.3 mm).

According to the disclosure, it was found that the aspect ratio (AR) of a strut of a scaffold may be between about 0.8 and 1.4, the AR of a link may be between about 0.4 and 0.9, or the AR of both a link and a strut may between about 0.9 and 1.1, or about 1. Aspect ratio (AR) is defined as the ratio of width to thickness. Thus, for a strut having a width of 0.0116 and a wall thickness of 0.011, the AR is 1.05.

According to the disclosure, the radial strength of a balloon expanded polymer scaffold having crush recoverability has a radial strength of greater than about 0.3 N/mm, or between about 0.32 and 0.68 N/mm, and a radial stiffness of greater than about 0.5 N/mm or between about 0.54 N/mm and 1.2 N/mm. According to the disclosure, a crush-recoverable scaffold has these ranges of stiffness and strength for a scaffold having a wall thickness of about 0.008″ to 0.014″ and configured for being deployed by a 6.5 mm non-compliant balloon from about a 2 mm crimped profile, or deployed to a diameter of between about 6.5 mm and 7 mm from about a 2 mm crossing profile on a balloon catheter.

A biodegradable polymer such as PLLA (and polymers generally composed of carbon, hydrogen, oxygen, and nitrogen) is radiolucent with no radiopacity. It is desirable, however, for a scaffold to be radiopaque, or fluoroscopically visible under x-rays, so that accurate placement within the vessel may be facilitated by real time visualization of the scaffold body, preferably the end rings. A cardiologist or interventional radiologist typically will track a delivery catheter through the patient's vasculature and precisely place the scaffold at the site of a lesion using fluoroscopy or similar x-ray visualization procedures. For a scaffold to be fluoroscopically visible, it should be more absorptive of x-rays than the surrounding tissue. Radiopaque materials in a scaffold may allow for its direct visualization. One way of including these materials with a biodegradable polymer scaffold is by attaching radiopaque markers to structural elements of the scaffold, such as by using techniques discussed in U.S. Publication No. 2007/0156230, herein incorporated by reference in its entirety. However, in contrast to other stents or scaffolds, a biodegradable, bioabsorbable, bioresorbable, or bioerodable, and peripherally implanted scaffold having crush recoverability according to the disclosure, has special requirements.

There is the need of maintaining a desired stiffness property in the vicinity of the marker-holding material (marker structure) without increasing the minimum crimped diameter, e.g., Dₘᵢₙ. The marker-holding material should not interfere with the extremely-limited space available for achieving the required crossing profile or delivery diameter for the crimped scaffold on the delivery catheter, particularly in the case of a scaffold that has a diameter reduction of 300-400% or more when crimped from the starting, pre-crimp diameter to the delivery diameter, and/or where the target delivery diameter is about at most a theoretical minimum diameter (Dₘᵢₙ) for the scaffold. It has been found that in order to be capable of achieving a desired delivery diameter, e.g., a 300-400% or more diameter reduction during crimping, the marker material (when located on a link) should not interfere with the folding of the struts forming rings of the scaffold. However, when addressing this need without consideration for the effect on radial stiffness, it was found that there was an unacceptable loss in stiffness in the vicinity of the marker structure.

Additional aspects of the radiopaque marker, including its location on the scaffold, can be found in U.S. Publication No. 2022/0226133, herein incorporated by reference in its entirety.

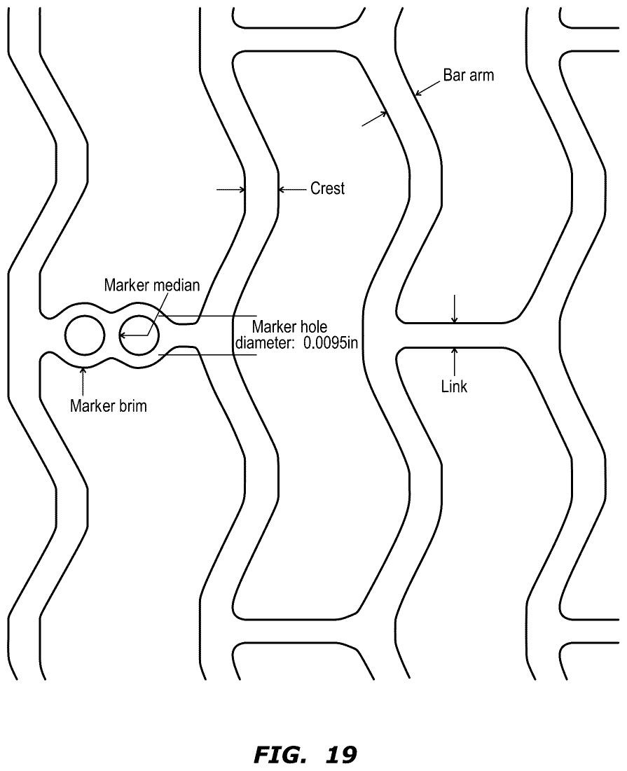

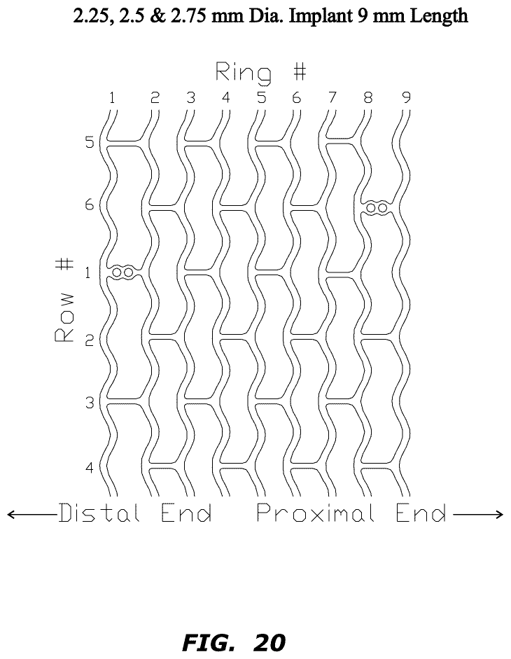

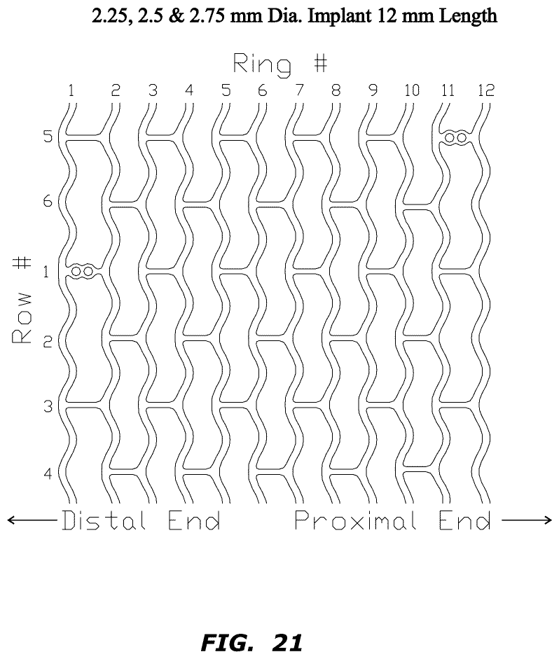

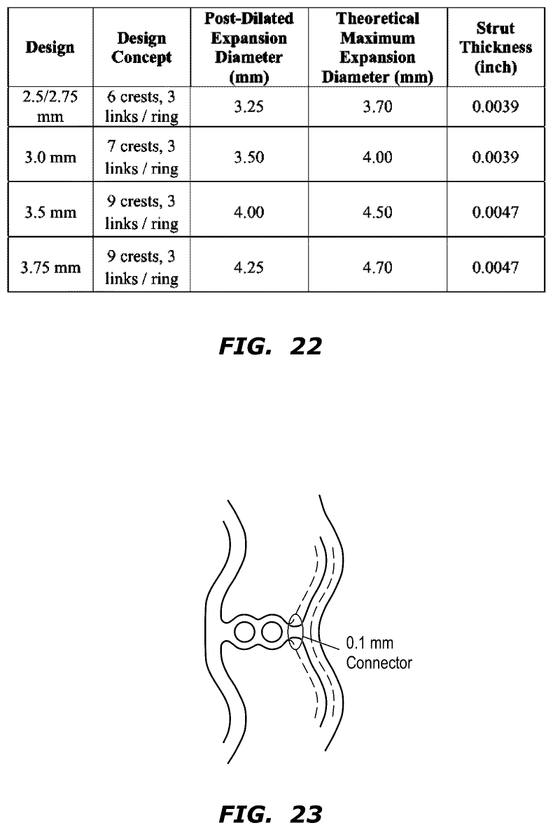

Additional exemplary polymer scaffolds are illustrated in FIGS. 19-33. For example, as shown in FIGS. 20-21, a scaffold may comprise a 6-crest, 3-link/ring pattern which has a 3.7 mm theoretical maximum expansion (TME) and is designed to be cut on a 3.47 mm outer diameter (OD) tube. To improve radial strength, this scaffold design has a wider strut width at the crests. Furthermore, the rings with marker beads have a narrower strut width than those of the other rings to maintain the theoretical minimum crimp of the design, thereby reducing the possibility of these rings having overlapped struts during crimping. The scaffold may optionally incorporate a connector between the distal marker beads and the Y crest of the 2nd distal ring (as shown in FIG. 23).

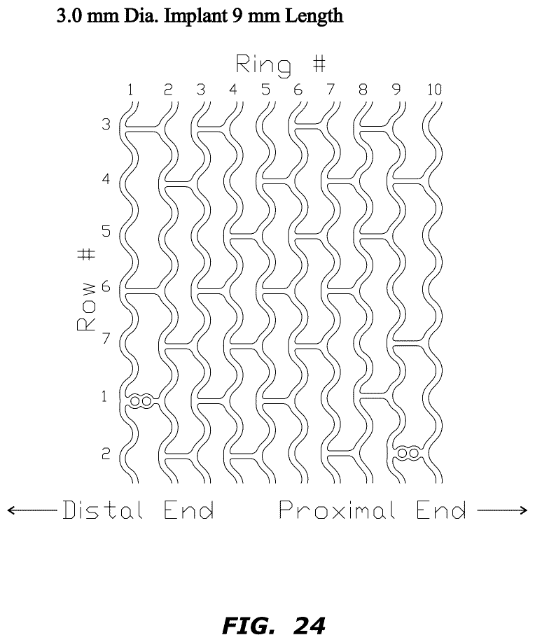

Another exemplary embodiment, shown in FIGS. 24-25, comprises a 7-crest, 3-link/ring pattern which has a 4.0 mm TME and is designed to be cut on a 3.47 mm outer diameter tube. To improve radial strength, this scaffold design has a wider strut width at the crests. Furthermore, the rings with marker beads have a narrower strut width than those of the other rings to maintain the theoretical minimum crimp of the design, thereby reducing the possibility of these rings having overlapped struts during crimping. The scaffold may optionally incorporate a connector between the distal marker beads and the Y crest of the 2nd distal ring, depicted in FIG. 23.

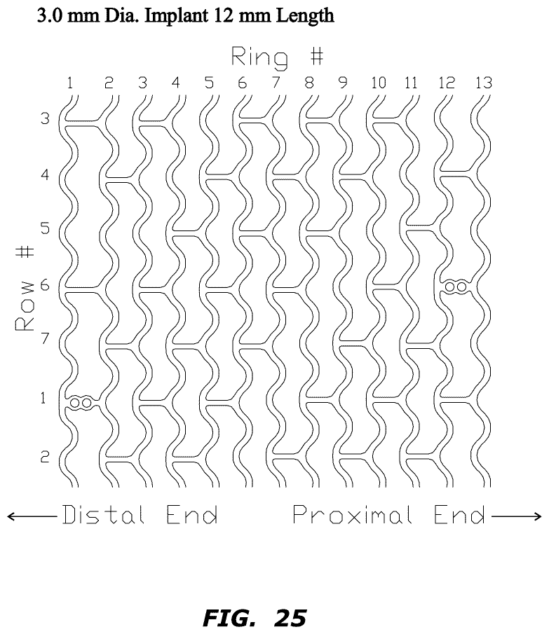

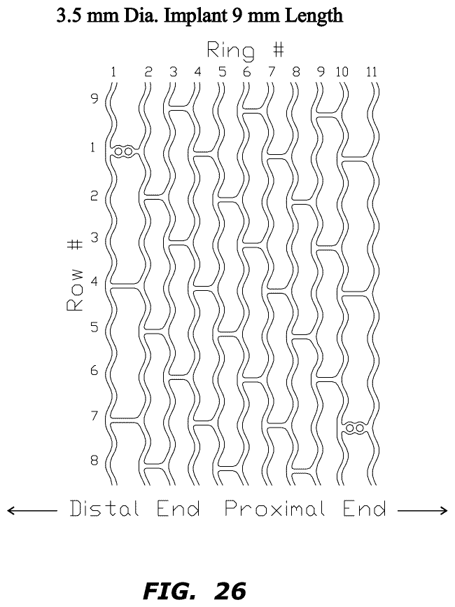

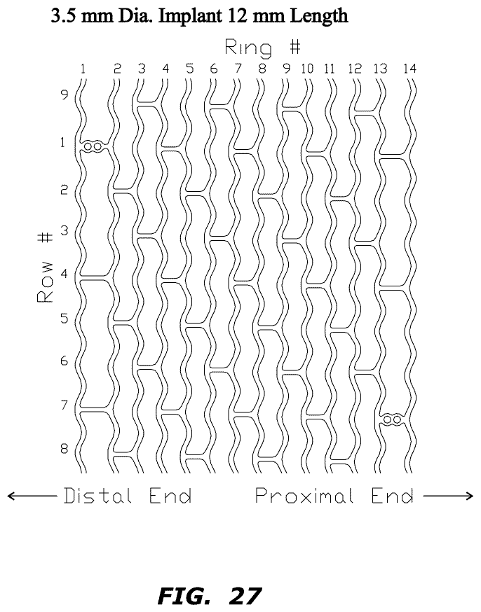

Another exemplary embodiment is shown in FIGS. 26-27. The scaffold design is a 9-crest, 3-link/ring pattern and is designed to be cut on a 4.25 mm OD tube. To improve radial strength, this scaffold design has a wider strut width at the crests. Furthermore, the rings with marker beads have a narrower strut width than those of the other rings to maintain the theoretical minimum crimp of the design, thereby reducing the possibility of these rings having overlapped struts during crimping.

The theory behind the relationship between radial strength and TME is as follows:
Bending stiffness is inversely proportional to bar arm length.
A scaffold design with smaller TME and correspondingly shorter bar arm length provides greater resistance to bending during radial compression and thus has a higher radial strength.
For the same strut thickness and width dimensions between two scaffolds, the smaller TME scaffold results in one more ring for most scaffold lengths (except for the 12 mm and 15 mm lengths). Higher number of rings per length is expected to boost radial strength.
A 4.5 mm TME provides a 0.25 mm expansion safety margin over the post-dilated expansion diameter which is in alignment with the expansion safety margins of the other designs.

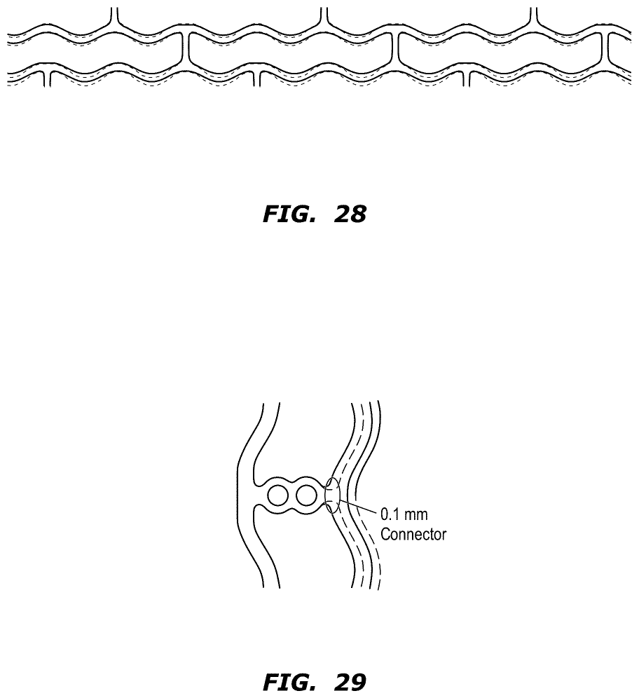

FIG. 28 illustrates the TME difference between smaller TME scaffold (in red) and the larger TME scaffold (in black).

The scaffold may be modified to incorporate a connector between the distal marker beads and the Y crest of the 2nd distal ring (depicted in FIG. 29).

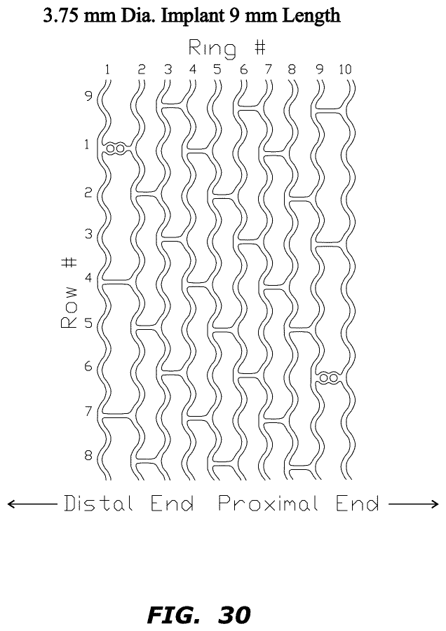

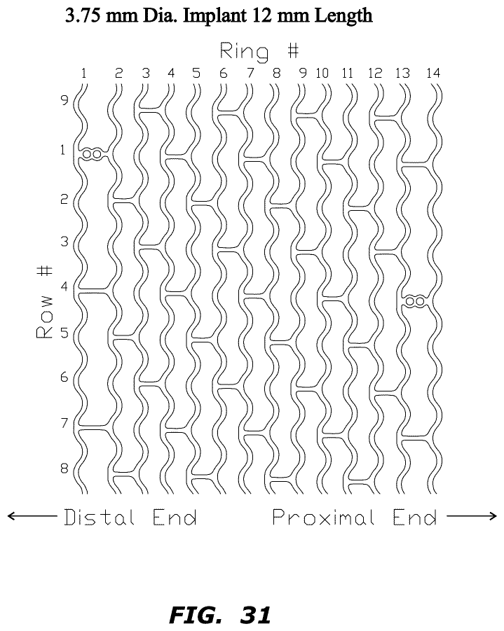

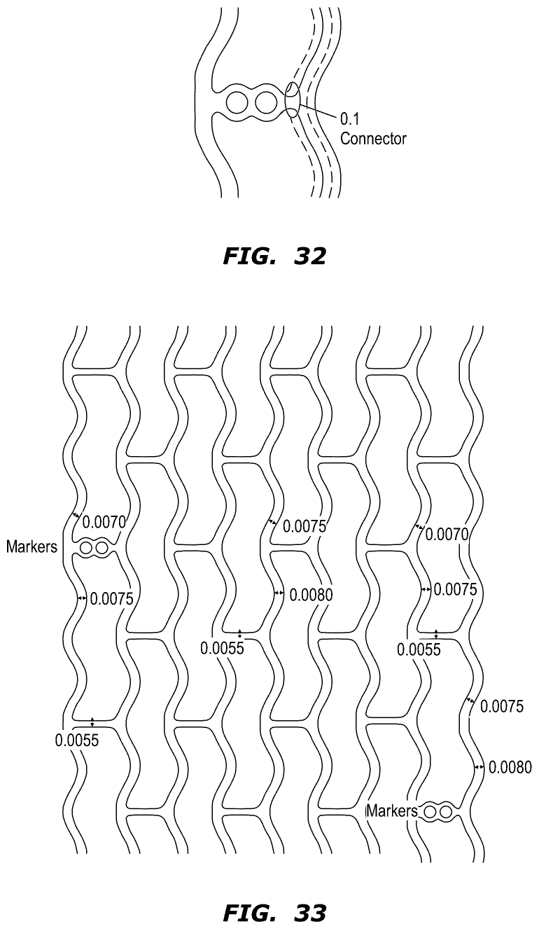

Another exemplary scaffold is shown in FIGS. 30-31. The scaffold design is a 9-crest, 3-link/ring pattern which has a 4.7 mm TME and is designed to be cut on a 4.25 mm OD tube. To improve radial strength, this scaffold design has a wider strut width at the crests. Furthermore, the rings with marker beads have a narrower strut width than those of the other rings to maintain the theoretical minimum crimp of the design, thereby reducing the possibility of these rings having overlapped struts during crimping. The scaffold may be modified to to incorporate a connector between the distal marker beads and the Y crest of the 2nd distal ring (depicted in FIG. 32).

An exemplary depiction of scaffold strut dimensions (in inches) for the embodiment depicted in FIGS. 20-21 is shown in FIG. 33. One of skill in the art will appreciate that a scaffold according to the present disclosure may deviate from these dimensions without departing from the spirit of the invention.

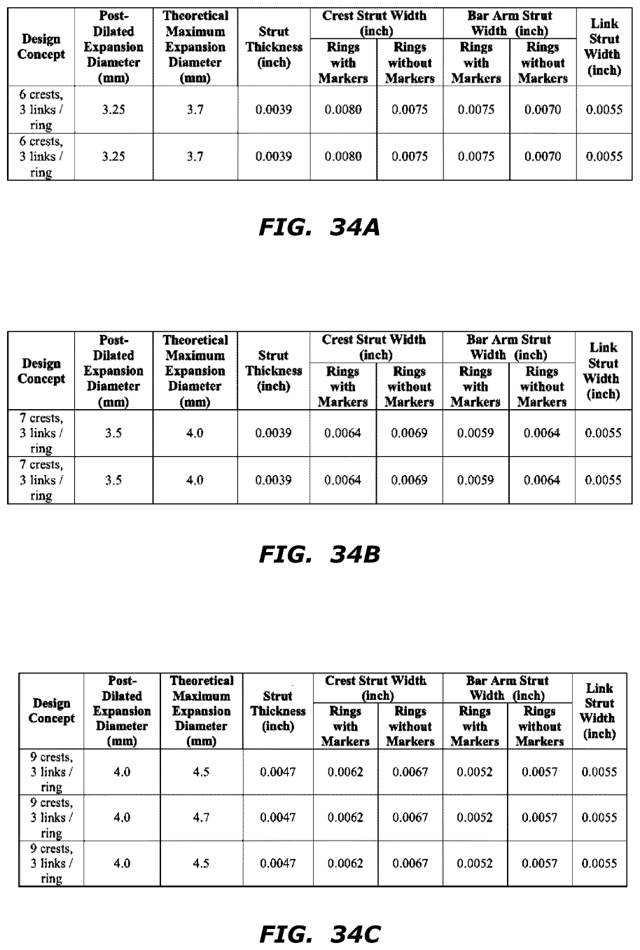

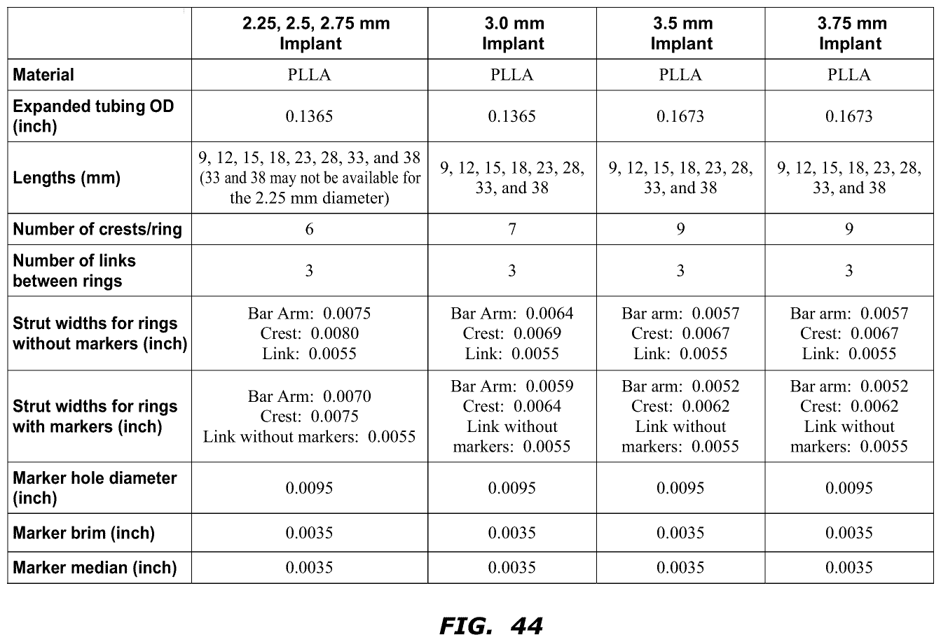

FIGS. 34A-34D illustrate characteristics of the exemplary scaffolds depicted in FIGS. 20-21, FIGS. 24-25, FIGS. 26-27, and FIGS. 30-31, respectively. FIG. 44 lists additional features of the exemplary polymeric scaffolds depicted in FIGS. 20-21, FIGS. 24-25, FIGS. 26-27, and FIGS. 30-31.

### IV. Design Process

As mentioned earlier, the problem may be stated in general terms as achieving the right balance among three competing design drivers: radial strength/stiffness verses toughness, in-vivo performance verses compactness for delivery to a vessel site, and crush recovery verses radial strength/stiffness.

Embodiments having patterns 200 or 300 were found to produce desired results with particular combinations of parameters disclosed herein, or readily reproducible in light of the disclosure. It will be recognized that there were no known predecessor balloon-expandable stents having adequate crush recovery to use as a guide (indeed, the art had discouraged such a path of development for a peripheral stent). As such, various polymer scaffold combinations were fabricated and the following properties evaluated to understand the relationships best suited to achieve the following objectives:

crush recoverability of the scaffold without sacrificing a desired minimal radial stiffness and strength, recoil, deploy-ability and crimping profile;

acute recoil at deployment—the amount of diameter reduction within ½ hour of deployment by the balloon;

delivery/deployed profile—i.e., the amount the scaffold could be reduced in size during crimping while maintaining structural integrity;

in vitro radial yield strength and radial stiffness;

crack formation/propagation/fracture when crimped and expanded by the balloon, or when implanted within a vessel and subjected to a combination of bending, axial crush, and radial compressive loads;

uniformity of deployment of scaffold rings when expanded by the balloon; and

pinching/crushing stiffness.

These topics have been discussed earlier. Additional examples and conclusions on the behavior of a scaffold according to the disclosure, so as to gain additional insight into aspects of the disclosed embodiments, can be found in U.S. Publication No. 2022/0226133, herein incorporated by reference in its entirety. Ultimately, a scaffold was designed (in accordance with the disclosure) having the desired set of scaffold properties while maintaining good crush recovery properties after a 50% pinch deformation, which refers to the scaffold's ability to recover its outer diameter sufficiently, e.g., to about 90-95%, following a crushing load that depresses the scaffold to a height equal to about 50% of its undeformed height.

The pinching stiffness (as opposed to the radial stiffness) is most influenced by, or most sensitive to, changes in the wall thickness of the scaffold. As the wall thickness increases, the pinching stiffness increases. Moreover, the crush recoverability of a scaffold is most affected by the stresses created at the regions that deflect outward the greatest in response to the applied load. As the wall thickness is increased, the crush recoverability decreases due to an increased concentration of strain energy at the outwardly deflected ends of the scaffold. A design for a crush recoverable scaffold, therefore, should balance the wall thickness for increased pinching stiffness against the reduction in crush recoverability resulting from an increased pinching stiffness. Similarly, although radial stiffness is less affected by changes in wall thickness (since loads are more predominantly in-plane loading as opposed to out of plane during pinching) when wall thickness is altered to affect crush recoverability, the radial stiffness should be taken into consideration. Radial stiffness changes when the wall thickness changes.

In a preferred embodiment, it was found that for a 9 mm scaffold pre-crimp diameter, a wall thickness of between 0.008″ and 0.014″, or more narrowly 0.008″ and 0.011″, provided the desired pinching stiffness while retaining 50% crush recoverability. More generally, it was found that a ratio of pre-crimp (or tube) diameter to wall thickness of between about 30 and 60, or between about 20 and 45, provided 50% crush recoverability while exhibiting a satisfactory pinching stiffness and radial stiffness. And in some embodiments, it was found that a ratio of inflated diameter to wall thickness of between about 25 and 50, or between about 20 and 35, provided 50% crush recoverability while exhibiting a satisfactory pinching stiffness and radial stiffness.

Wall thickness increases for increasing pinching stiffness may also be limited to maintain the desired crimped profile. As the wall thickness is increased, the minimum profile of the crimped scaffold can increase. It was found, therefore, that a wall thickness may be limited both by the adverse effects it can have on crush recoverability, as just explained, as well as an undesired increase in crimped profile.

### V. Testing

Results from various tests conducted on scaffolds and stents for purposes of measuring different mechanical properties and making comparisons between the properties of the stents and scaffolds can be found in U.S. Publication No. 2022/0226133, herein incorporated by reference in its entirety.

In accordance with the disclosure, a crush-recoverable polymer scaffold (having adequate strength and stiffness properties) has a greater than about 90% crush recoverability when crushed by an amount equal to about 33% of its starting diameter, and a greater than about 80% crush recoverability when crushed by an amount equal to about 50% of its starting diameter, following an incidental crushing event (e.g., less than one minute). A crush-recoverable polymer scaffold has a greater than about 90% crush recoverability when crushed by an amount equal to about 25% of its starting diameter, and a greater than about 80% crush recoverability when crushed by an amount equal to about 50% of its starting diameter for longer duration crush periods (e.g., between about 1 minute and five minutes, or longer than about 5 minutes).

It was found that a larger pre-crimp diameter relative to the intended inflated diameter exhibited much less recoil when deployed to 6.5 mm. It is believed that the memory of the material, formed when the deformed tube was made, reduced the acute recoil. However, when the starting diameter exceeds a threshold, it becomes infeasible to maintain the desired crimped profile. It was found that a 9 mm tube size produced acceptable results, in that there was less recoil and a crimped profile of about 2 mm could still be obtained.

It was also found that a 9 mm starting diameter scaffold (in combination with other scaffold dimensions) could be reduced down to 2 mm, then expanded to the 7.4 mm inflated diameter, without excessive cracking or fracture.

As discussed earlier, unlike a metal stent, a design for a polymer scaffold should take into consideration its fracture toughness during implantation within a vessel. For a scaffold located within a peripheral artery, the types of loading encountered are generally more severe in terms of bending and axial loading than a coronary scaffold, in addition to the pinching or crush forces experienced by the scaffold, due to the scaffold's proximity to the surface of the skin and/or its location within or near an appendage of the body. See, e.g., Nikanorov, Alexander, M. D. et al., Assessment of self-expanding Nitinol stent deformation after chronic implantation into the superficial femoral artery, herein incorporated by reference in its entirety.

As is known in the art, a scaffold designed to have increased radial stiffness and strength properties does not generally also exhibit the fracture toughness needed for maintaining structural integrity. The need to have a peripherally implanted polymer scaffold with adequate fracture toughness refers both to the need to sustain relatively high degrees of strain in or between struts and links of the scaffold and to sustain repeated, cyclical loading events over a period of time, which refers to fatigue failure.

The methods of manufacture, discussed earlier, of the tube from which the scaffold is formed are intended to increase the inherent fracture toughness of the scaffold material. Additional measures may, however, be employed to reduce instances of fracture or crack propagation within the scaffold by reducing the stiffness of the scaffold in the links, or by adding additional hinge points or crown elements to the ring. Alternatively, or in addition, pre-designated fracture points can be formed into the scaffold to prevent fracture or cracks from propagating in the more critical areas of the scaffold. Examples are provided.

As mentioned above, a peripherally implanted polymer scaffold is subjected to a combination of radial compressive, pinching or crushing, bending, and axial compression loads. Test results indicate that a majority of cracks occur in the struts forming a ring, as opposed to the links connecting rings for a peripherally implanted polymer scaffold. Indeed, while bench data may suggest that a scaffold is quite capable of surviving cyclical radial, bending, and axial loadings when implanted in a peripheral vessel, when the scaffold is in-vivo subjected to combined axial, flexural, and radial loading in a peripheral vessel, there is nonetheless unacceptable crack formation, fracture, or significant weakening in radial strength.

With this in mind, alternative embodiments of a scaffold pattern seek to weaken, or make more flexible, the scaffold in bending and axial compression without significantly affecting the radial strength or stiffness of the scaffold. By making links connecting rings more flexible, relative movement between a ring and its neighbor, which occurs when a scaffold rings are not axially aligned with each other and the scaffold is placed in bending or axial compression (e.g., when the scaffold resides in a curved vessel), does not produce as high of a loading between the ring and its neighbor since the link tends to deflect more in response to the relative movement between the rings, rather than transfer the load directly from one ring to another.

Referring to an alternative embodiment of pattern 200, a scaffold is constructed according to the pattern depicted in FIGS. 6A and 6B. Pattern 400 is similar to pattern 200 except that a link 434/440 connecting the rings 212 is modified to create greater flexibility in the scaffold in bending and axial compression (or tension). Referring to FIG. 6B, the link 434 includes a first portion 435 having a first moment of inertia in bending (MOI₁,) nearest a Y-crown of a ring and a second portion 438 having a second moment of inertia (MOI₂) in bending nearest a W-crown of the ring, where MOI₁<MOI₂. Additionally, a U-shaped portion 436 is formed in the portion 435 to create, in effect, a hinge or articulation point to reduce bending stiffness further. The U-shape portion 436 opens when the ring 212 rotates clockwise in FIG. 6B. As such, the link is very flexible in clockwise bending since the bending stiffness about the hinge 436a is very low. For counterclockwise rotation, the ends of the U-shaped portion abut, which in effect negates the effect of the hinge 436a.

To construct a scaffold that is equally flexible for both clockwise and counterclockwise bending of the scaffold, the U-shaped portions 434 may be removed so that the increased flexibility is provided solely by the reduced MOI portions of the links, such as by replacing the U-shaped portion 436 in FIG. 6B with a straight section having a reduced MOI.

In another embodiment a reduced MOI may be achieved by increasing the distance between each ring, or preferably every other ring. In another alternative, the pattern 400 includes a link with opposing “U” shaped portions or an “S” portion. Additional examples toward this end can be found in US Publication No. 2022/0226133, herein incorporated by reference in its entirety. These examples show links having variable MOIs, either by shaping the link as the pattern is cut from a tube or by modifying the link after the scaffold has been cut from the tube.

Additionally and/or alternatively, greater fatigue and/or fracture toughness may be achieved by modifying the struts of the ring. FIG. 7 illustrates a pattern similar to pattern 300, except that the rings 450 are formed by curved struts 452 connected at crowns 451. In this example, the struts 452 have a shape approximating one sinusoidal period. By replacing the straight struts of FIG. 3 with sinusoidal struts, there are essentially additional hinge points created in the ring.

In another aspect of the disclosure, there is a scaffold pattern having rings formed by closed cells. Each of the closed cells of a ring share a link element that connects the longitudinally spaced, and circumferentially extending, strut portions of the closed cell. Each of these closed cell rings are interconnected by a connecting link e.g., links, 434, 442, 450, 452 or 454, having a reduced bending moment of inertia (MOI) to reduce the flexural rigidity of the structure connecting the closed cell rings. Alternatively, the connecting link can include a pre-designated fracture point, such as by forming an abrupt change in geometry near a high strain region. Returning again to FIG. 6A, the scaffold pattern depicted has links 440a connected to each closed cell ring. For each closed cell 204 there is a first and second connecting link, which are co-linear with each other. The first link has a MOI₁, disposed adjacent the crown and the second link has a MOI₂, disposed distal the crown to produce the pattern shown in FIG. 6A. Alternatively, the links connecting the closed cell rings may have the MOI₁ disposed equidistant from the interconnected closed cell rings.

According to an additional aspect of the disclosure, there is a scaffold that includes pre-designated fracture points in the links connecting rings. The fracture points are intended to relive the inter-ring loading through crack formation in the links connecting rings. Since the loading on a crown is reduced or eliminated when there is sufficient crack propagation through the link (load cannot transfer across a crack), by including a pre-designated crack location, one may maintain the integrity of the ring structure at the expense of the links (e.g., links 450, 452) in the event in-vivo loading exceeds the design, particularly with respect to fatigue loading. According to this aspect of the disclosure, a link has a reduced MOI near a high strain region and includes an abrupt geometry change—e.g., about 90 degrees mid-span. These pre-designated fracture points in the scaffold may extend between closed cell rings, as described above, or between each ring strut.

A metal stent may be cut from a tube that is between the deployed and crimped diameters. As such, the spacing between struts is greater and the stent is more easily compressed on the balloon because the stent pre-crimp has a diameter closer to the crimped diameter. A polymer scaffold, in contrast, may be cut from a diameter tube equal to or greater than the deployed state. This means there is more volume of material that should be packed into the delivery profile for a polymer scaffold. A polymer scaffold, therefore, has more restraints imposed on it, driven by the crimped profile and starting tube diameter, that limits design options on strut width or thickness.

A well-known design requirement for a vessel supporting prosthesis, whether a stent or scaffold, is its ability to maintain a desired lumen diameter due to the inward radial forces of the lumen walls including the expected in vivo radial forces imparted by contractions of the blood vessel. The radial stiffness and radial strength of the scaffold is influenced by the width of struts, crown radii and angles, length of ring struts extending between crowns and valleys, the number of crowns, and the wall thickness of the scaffold. The latter parameter (wall thickness) influences the pinching stiffness, as explained earlier. During the design process, therefore, this parameter was altered to affect pinching stiffness and crush recoverability, although it also influences radial stiffness. In order to affect the radial stiffness, one or more of the foregoing parameters (crown angle, crown radius, ring strut length, crown number, and strut width) may be varied to increase or decrease the radial stiffness.

The relationships between radial stiffness and above-mentioned parameters are well known. However, the relationship of these stiffness-altering parameters to crush recoverability of a balloon-expandable stent, much less a balloon-expandable scaffold, is not well known, if known at all, in the existing art. Accordingly, the design process required the constant comparison or evaluation among radial stiffness, pinching stiffness, and crush recoverability (assuming the changes did not also introduce yield or fracture problems during crimping and deployment) when the stiffness parameters were altered to determine whether these and related scaffold properties could be improved upon without significant adverse effects to crush recoverability.

In general, the more crowns, the more compliant becomes the scaffold radially and the higher the crown angle, the less radially complaint becomes the scaffold. More details on the above-mentioned relationships can be found in U.S. Publication No. 2022/0226133, herein incorporated by reference in its entirety.

According to one aspect of the disclosure, a crush recoverable scaffold has: a ratio of pinching stiffness to radial stiffness of between about 4 to 1, 3 to 1, or more narrowly about 2 to 1; a ratio of pinching stiffness to wall thickness of between about 10 to 70, or more narrowly 20 to 50, or still more narrowly between about 25 and 50; and a ratio of scaffold inflated diameter to pinching stiffness of between about 15 and 60, or more narrowly between about 20 to 40.

According to another aspect of the disclosure a crush-recoverable scaffold has a desirable pinching stiffness to wall thickness ratio of 0.6-1.8 N/mm².

According to another aspect of the disclosure, a crush-recoverable scaffold has a desirable pinching stiffness to wall thickness*tube diameter ratio of 0.08-0.18 N/mm³.

### VI. Below the Knee (BTK) Treatment of Peripheral Artery Disease (PAD)

As discussed above, polymer scaffolds in accordance with the present disclosure are useful for below the knee (BTK) treatment of peripheral artery disease (PAD).

In the prior art, several trials have evaluated methods for revascularization in the infrapopliteal circulation in an attempt to avoid the relatively poor patency outcomes seen with angioplasty, even where angioplasty is the current state of the art treatment for such conditions. However, most of these methods have failed because of the complex nature of the atherosclerotic disease and the difficulty of maintaining patency in both the short term and the long term. The challenges associated with infrapopliteal artery revascularization include extensive medial calcinosis, long lesion lengths, acute lesion recoil, and a predilection for flow-limiting dissection after angioplasty. Various methods have not shown efficacy with respect to the maintenance of long-term patency and the reduction of undesirable long-term clinical events, such that reintervention and amputation are still too often required. Significantly improved results may depend both on the mechanical properties of the stent, an antiproliferative coating, and careful screening of patients for potential implantation, and new methods of post-implantation evaluation.

Drug-eluting devices that inhibit neointimal hyperplasia have not been used routinely for the treatment of infrapopliteal artery disease. In numerous trials of drug-coated balloons and drug-eluting scaffolds and stents, the treatment has not resulted in greater patency than angioplasty or has had practical limitations. Of all the available approaches, the use of coronary drug-eluting stents with sirolimus analogues in below-the-knee interventions has shown the most promise for maintaining primary patency. However, the permanent nature of these metal implants comes with significant disadvantages, and has made some clinicians wary of their routine use.

Surprisingly, the applicant found that subjects presenting with chronic limb threatening ischemia (CLTI) associated with either ischemic rest pain (Rutherford-Becker class 4) or minor tissue loss (Rutherford-Becker class 5) and which had infrapopliteal artery stenosis or occlusion, could be successfully treated by placement of a polymer scaffold such as those described herein in a peripheral, below the knee artery of the subject, with results that show significant improvement over state of the art balloon angioplasty treatments.

The polymeric scaffold used to treat PAD in subjects may optionally be coated with an active pharmaceutical agent. Such active pharmaceutical agent may include an anti-proliferative drug such as everolimus, sirolimus, other Limus drug, or any combination thereof.

Prior to insertion of the scaffold into the below the knee peripheral artery, pre-dilation may be performed. If pre-dilation is performed, such may be performed using a non-compliant balloon, preferably with about a 1:1 ratio of balloon diameter to vessel diameter. In an embodiment, successful pre-dilation may result in residual stenosis of less than 30% of the vessel diameter. Post-dilation may also be performed following the insertion of the scaffold to ensure vessel wall apposition. When performed, post-dilatation may be performed at high pressure (>16 atm) with a non-compliant balloon up to about 0.5 mm larger than the nominal scaffold diameter.

The length of the polymeric scaffold may be selected so as to cover a minimum of 2 mm of healthy reference vessel at both the proximal edge and the distal edge of the target lesion. By way of example, typical scaffold lengths may range from about 15 mm to about 100 mm, from about 20 mm to about 85 mm, from about 20 mm to about 60 mm, from about 30 mm to about 50 mm, or from about 20 mm to about 40 mm. In a specific embodiment, the scaffold length may be from about 18 mm to about 38 mm, or up to about 60 mm or up to about 85 mm. Multiple scaffolds may be used.

A bioresorbable, polymeric scaffold in accordance with this disclosure may be inserted into a peripheral artery below the knee, such as the infrapopliteal arteries (i.e., below the popliteal), such as the tibioperoneal trunk, peroneal artery, anterior tibial artery and/or posterior tibial artery, at the site of a target lesion of a subject. In accordance with the disclosure, discussed above, the scaffold may have a pattern of interconnected elements, the interconnected elements comprising struts and links. The resorbable scaffold may be inflated in 2 atm increments every 5 seconds until fully expanded.

In an exemplary embodiment, there are 3 keys steps involved in the implantation of the scaffold in the vessel: (1) pre-dilatation of the target lesion, (2) appropriate sizing of the vessel and scaffold (in relation to vessel diameter), and (3) post-dilatation of the scaffold. Pre-and post-dilation were discussed above. In terms of sizing, quantitative imaging may be employed for the assessment of the reference vessel diameter at baseline in order to allow selection of the appropriate scaffold size. Quantitative imaging may be employed to accurately measure and confirm appropriate vessel sizing (e.g., reference vessel diameter ≥2.5 mm).

The progress of treatment of the target lesion may be assessed by means of angiography performed in magnified orthogonal views and/or duplex ultrasound (DUS). Successful treatment immediately after the treatment method may be defined as follows: residual stenosis of less than 30% of the vessel diameter, a final number of runoff vessels that is equal to or greater than the number on initial angiography, the absence of residual dissection (defined as the absence of a clinically persistent or increased amount of contrast material outside the vessel lumen), and the absence of complications, such as distal embolization, perforation, or thrombosis.

The applicant tested an exemplary method in accordance with the disclosure against a standard of care treatment (balloon angioplasty) for the treatment of subjects with CLTI. The applicant found that, as compared to balloon angioplasty, performance of a treatment method such as those disclosed herein (using a polymeric scaffold), successful treatment immediately after the treatment method occurred in 91% of the subjects in the polymeric scaffold group as compared to 70% of the subjects in the balloon angioplasty group. Such an improvement represents a significant advancement in the art.

In addition to evaluating restenosis immediately following the treatment method, the longer-term success of the method may be evaluated at various time-points after the procedure using angiography and/or DUS. For example, follow-up angiography and/or DUS may be performed at one or more periodic follow up time periods. Examples of such include one or more of 30 days (+/−14 days), 3 months (+/−14 days), 6 months (+/−28 days), 1 year (+/−28 days), 2 years (+/−28 days), 3 years (+/−28 days), 4 years (+/−28 days), or 5 years (+/−28 days) after inserting the scaffold into the peripheral artery below the knee. Alternatively, angiography and/or DUS may be performed on an unscheduled basis to evaluate whether reintervention is needed. In the case where a DUS result is undiagnostic (results cannot be determined from the data), the DUS may be repeated, or angiography performed. Angiography and/or DUS may be performed in order to measure binary restenosis (i.e., whether restenosis is present or not, as a binary choice) at the site of the target lesion, wherein binary restenosis is defined as the presence of a hemodynamically significant restenosis of >50% identified through angiography, or peak systolic velocity ratio (PSVR) ≥2.0 as identified by DUS.

Success of the treatment method may be evaluated using a primary efficacy end point, which may be defined as freedom from the following events at 1 year after performance of the method: amputation above the ankle of the target limb, total (100%) occlusion of the target vessel, clinically driven revascularization of the target lesion, and binary restenosis of the target lesion, where binary restenosis is defined as the presence of restenosis of more than 50% of the vessel diameter on angiography or a peak systolic velocity ratio (PSVR) of 2.0 or more on duplex ultrasonography. The use of binary restenosis as an efficacy endpoint is useful because it resolves some issues faced by previous trials that had insufficient power to discern clinically relevant treatment effects of a test device as compared with the standard of care. Clinically-Driven Target Lesion Revascularization (CD-TLR) may be defined as repeat intervention on the target lesion due to recurrent symptoms and stenosis >70% as determined by angiography. Bailout with a metallic stent, due to acute closure or to achieve <30% stenosis during the index procedure, may be considered a CD-TLR. Recurrent symptoms associated with such a CD-TLR include delayed or worsening wound healing, new or recurrent wound at the treatment site, or worsening Rutherford-Becker class.

In the presence of abnormal reference peak systolic velocity (PSV), the following additional secondary criteria (correlating factors) may be used to identify target lesion stenoses >50% in severity:
(1) Focal increase in the absolute PSV at the area of visible plaque;
(2) Spectral broadening of the waveform at the area of stenosis;
(3) Post-stenotic turbulence (PST) and/or significant change in the waveform shape and/or reduction in velocity distal to the stenosis; and/or
(4) Review of the B-mode images for plaque burden (i.e., visible stenosis on B-mode imaging).
If the PSVR cannot be calculated, these secondary criteria may be used to determine whether stenosis is present.

If a subject undergoes both angiography and DUS at the same time point (same follow-up appointment), the results of angiography may be used as the primary determinant of whether stenosis is present.

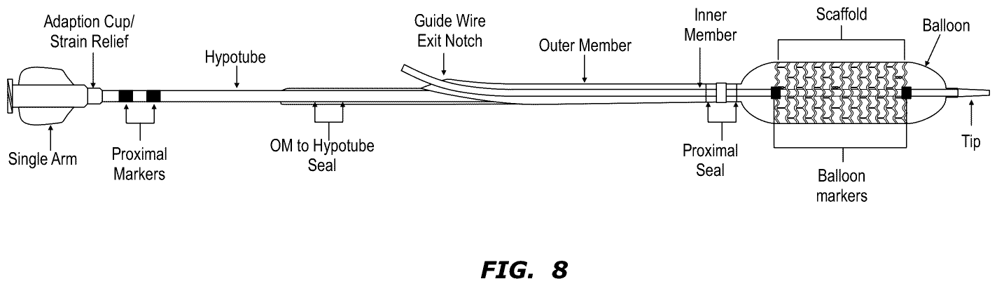

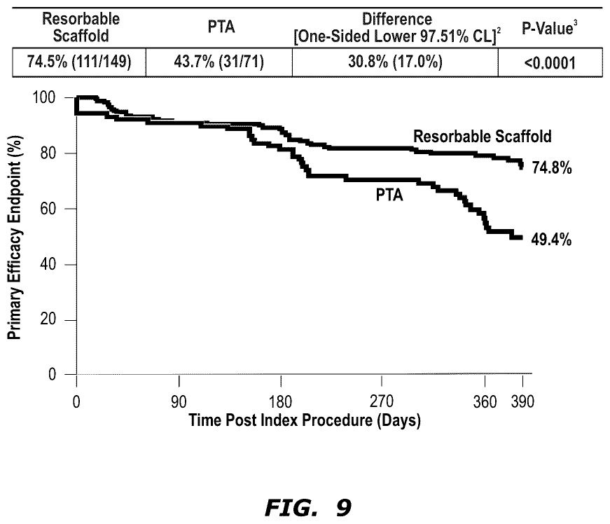

As mentioned above, the applicant compared an exemplary method in accordance with the disclosure against a standard of care treatment (e.g., balloon angioplasty) for the treatment of subjects with CLTI. The applicant employed a bioresorbable scaffold backbone comprised of 100% poly(L-lactide) (PLLA), which contained a coating comprised of the active pharmaceutical ingredient everolimus and bioresorbable poly(D,L-lactide) (PDLLA). The scaffold also comprised four platinum radiopaque markers of the same mass, two each at the proximal and distal ends of the scaffold for radiopacity. The polymeric scaffold was incorporated into an otherwise standard delivery system. An exemplary system, such as that employed by the applicant for such clinical trials, is illustrated in FIG. 8.

Within the clinical trial, the scaffold is bioresorbable, enabling gradual vessel remodeling and restoration of native anatomy over time. Pre-dilation of the lesion was employed. By way of example, operators were encouraged to use non-compliant balloons sized at a 1:1 ratio to the reference vessel diameter, aiming for residual stenosis of less than 30% prior to scaffold deployment. Post-dilatation was recommended using non-compliant balloons appropriately sized to remain within the scaffold margins. The maximum allowable scaffolded length per patient in the trial was 170 mm, and tandem lesions separated by less than 3 cm were treated as a single target lesion. Dual antiplatelet therapy (DAPT) was advised for a minimum of 12 months following scaffold implantation, followed by single antiplatelet therapy thereafter.

The exemplary method was specifically performed on screened subjects known to have at least a 70% stenosis at the site of the target lesion prior to insertion of the scaffold. The polymeric scaffold was placed into the proximal ⅔ of native infrapopliteal arteries for treatment of one or more lesions in each subject.

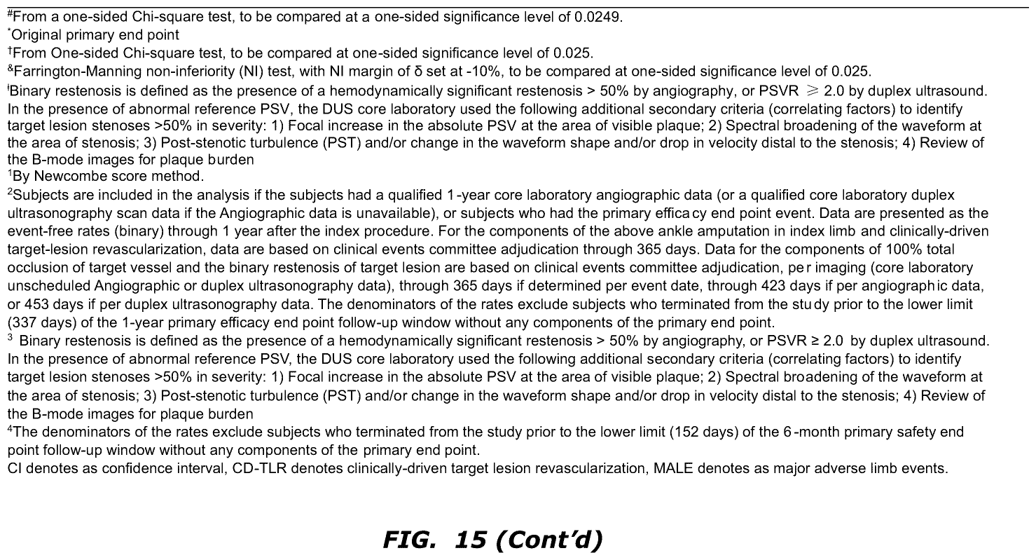

As shown in FIG. 9 (Kaplan-Meier estimate) and FIG. 15, the applicant found that, for the primary efficacy end point (freedom from each of: amputation above the ankle of the target limb, occlusion of the target vessel, clinically driven revascularization of the target lesion, and binary restenosis of the target lesion), evaluated at 1 year after insertion of the scaffold, was 74.5% in the polymeric scaffold group of subjects and 43.7% in the angioplasty group of subjects, with an absolute difference of about 30 percentage points (95% confidence interval). Because these end points were “freedom from” end points, these results indicate that the efficacy of the claimed methods are significantly superior to the standard of care method. Specifically, the results show that, among patients with CLTI and infrapopliteal artery disease, the incidence of freedom from amputation above the ankle of the target limb, occlusion of the target vessel, and clinically driven revascularization of the target lesion at 1 year was significantly higher among patients who received an everolimus-eluting resorbable polymeric scaffold than among those who received balloon angioplasty. The magnitude of the effect was increased when freedom from binary restenosis of the target lesion was included in the end point. Detailed results are shown in FIG. 15.

A primary safety end point for the claimed treatment methods may also be evaluated and may include freedom from (1) major adverse limb events at 6 months and (2) perioperative death. Major adverse limb events may be defined as amputation above the ankle of the target limb and major reintervention, which may be defined as any of new surgical bypass grafting, interposition grafting, thrombectomy, or thrombolysis. Perioperative death may be defined as death from any cause within 30 days after the procedure.

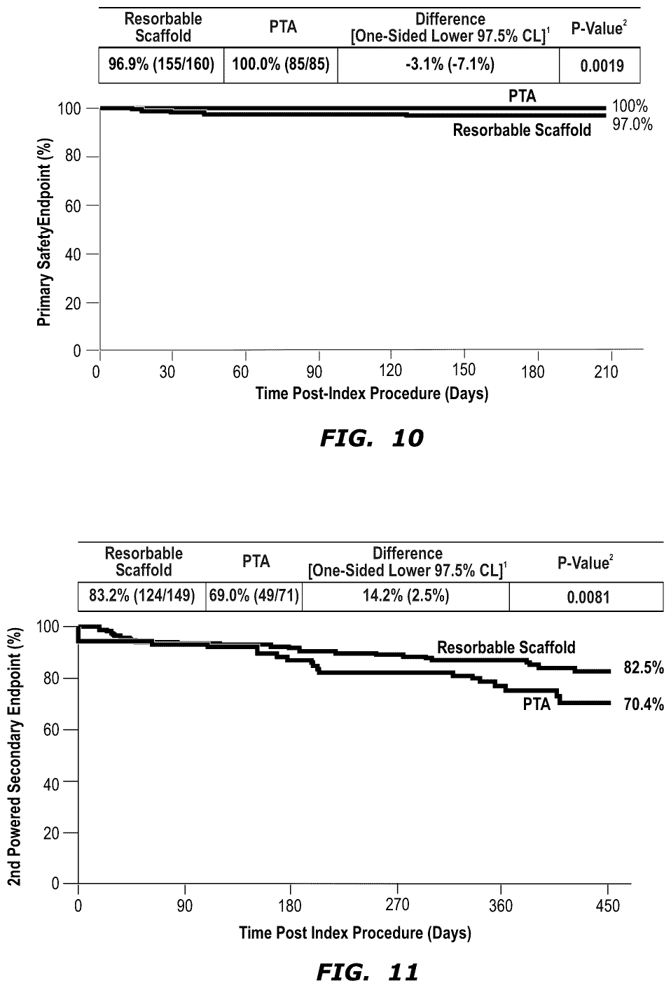

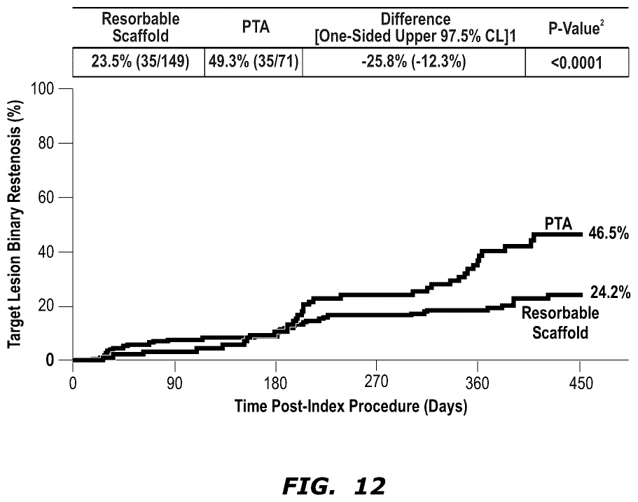

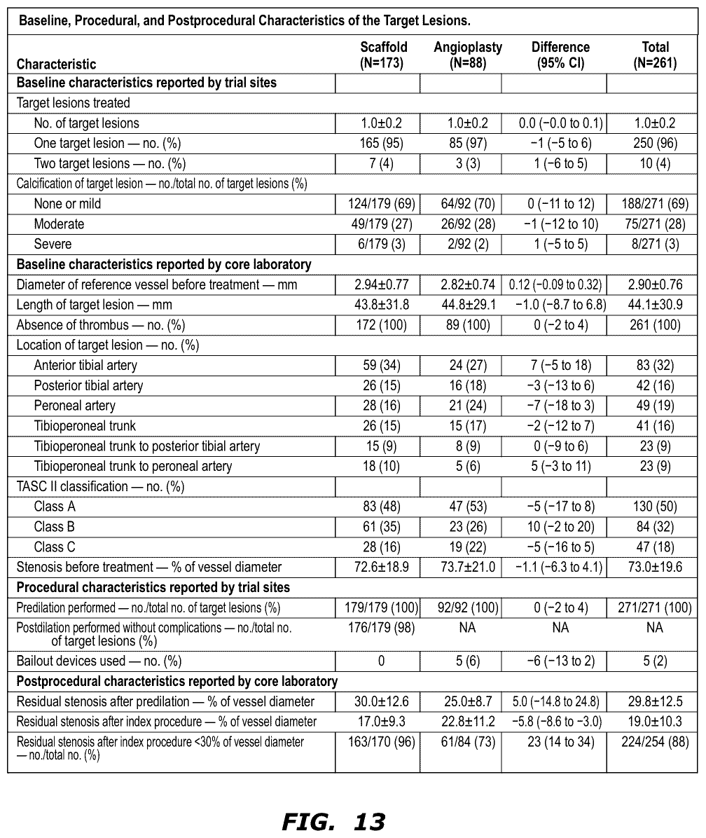

As shown in FIG. 10 (Kaplan-Meier estimate) and FIG. 15, the applicant found that the primary safety end point (freedom from major adverse limb events at 6 months and perioperative death) was observed in 155 of 160 patients in the polymeric scaffold group and 85 of 85 patients in the angioplasty group, with an absolute difference of −3.1 percentage points (95% CI; for noninferiority). These results indicate that the safety of the claimed methods is comparable to the standard of care method. In other words, the use of the polymeric scaffold was noninferior to balloon angioplasty. Noninferiority was defined as within −10 percentage points, in the presently described trials.

Secondary end points may also be evaluated for the claimed methods and may include (1) three factors of the primary efficacy end point (freedom from amputation above the ankle of the target limb, occlusion of the target vessel, and clinically driven revascularization of the target lesion) and (2) binary restenosis of the target lesion, both of which may be adjudicated at 1 year.

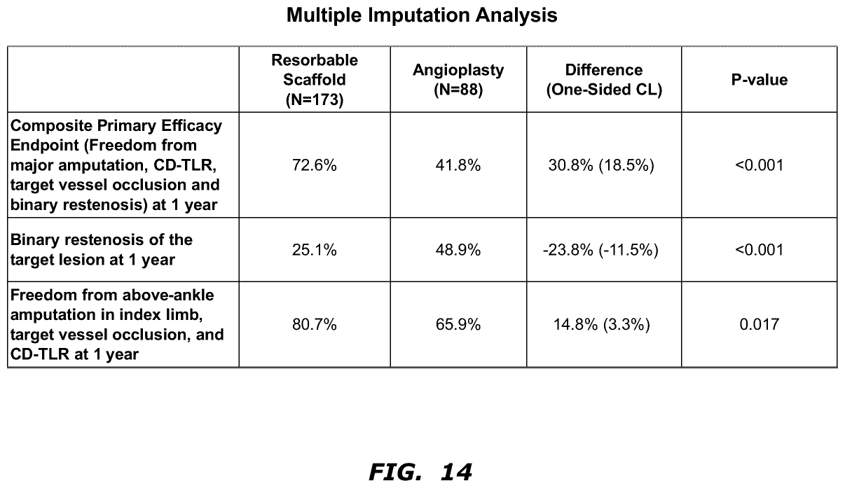

The applicant found that the first powered secondary end point (freedom from: amputation above the ankle of the target limb, occlusion of the target vessel, and clinically driven revascularization of the target lesion at 1 year) was observed in 124 of 149 patients in the polymeric scaffold group and 49 of 71 patients in the angioplasty group. These results are shown in FIG. 11 (Kaplan-Meier estimate), and FIG. 15. The second powered secondary end point (binary restenosis of the target lesion at 1 year) was observed in 35 of 149 patients in the scaffold group and 35 of 71 patients in the angioplasty group. These results are shown in FIG. 12 (Kaplan-Meier estimate) and FIG. 15. The results for the secondary end points were significant, although the magnitude of the effect was smaller than that for the primary efficacy end point. Nevertheless, the secondary end point results confirm that the efficacy of the claimed methods is superior to, the standard of care method.

Wound healing may also be monitored as a way of assessing the success of the treatment methods. All wounds may be indexed, including first occurrence of new wounds, and analyzed quantitatively, for etiology, and for healing. The applicant found that wound healing was observed in 37 of 83 patients (45%) in the polymeric scaffold group by 1 year, with a mean time to healing of 196.7±130.1 days. Wound healing was observed in 25 of 45 patients (56%) in the angioplasty group by 1 year, with a mean time to healing of 187.6±122.7 days. Thus, based on wound healing, the claimed methods are on par with the standard of care method.

The following patient-reported outcomes (analyzed as informational endpoints at baseline, 30 days, 3 months, 6 months, 1 year and/or other time period) may also be used to evaluate the efficacy and safety of using the disclosed polymeric scaffolds for the treatment of CLTI:
(1) Overall health status using the EQ-5D-5L (EuroQoL-5D-5L) questionnaire,
(2) Walking capacity using WIQ (Walking Impairment Questionnaire), and/or
(3) Disease-specific health status using PAQ (Peripheral Artery Questionnaire).

More detail regarding a process for BTK treatment of PAD using polymeric scaffolds, and evaluating the efficacy of those methods, can be found in applicant's publications of these clinical trial results, i.e., see Varcoe, et al., Drug-Eluting Resorbable Scaffold versus Angioplasty for Infrapopliteal Artery Disease, N ENGL J MED, 390:9-19 (2024), and Ramon L. Varcoe et al., on behalf of the LIFE-BTK Investigators, Primary Outcomes of the Esprit™ BTK Drug-Eluting Resorbable Scaffold for the Treatment of Infrapopliteal Lesions—The LIFE-BTK Trial, Transcatheter Cardiovascular Therapeutics (TCT) 2023 conference. Each of the foregoing is herein incorporated by reference in its entirety.

Based on the results of the primary efficacy end point and secondary end points evaluated, bioresorbable polymeric scaffolds according to the disclosure are superior to standard of care treatments, such as balloon angioplasty, for the treatment of CLTI. In terms of the safety endpoint, bioresorbable polymeric scaffolds such as those disclosed herein are non-inferior to balloon angioplasty. For example, the measured primary efficacy endpoint difference of +30.8% (see FIG. 15) for use of the bioresorbable polymeric scaffold as compared to ballon angioplasty in treating CLTI is remarkable, and very advantageous.

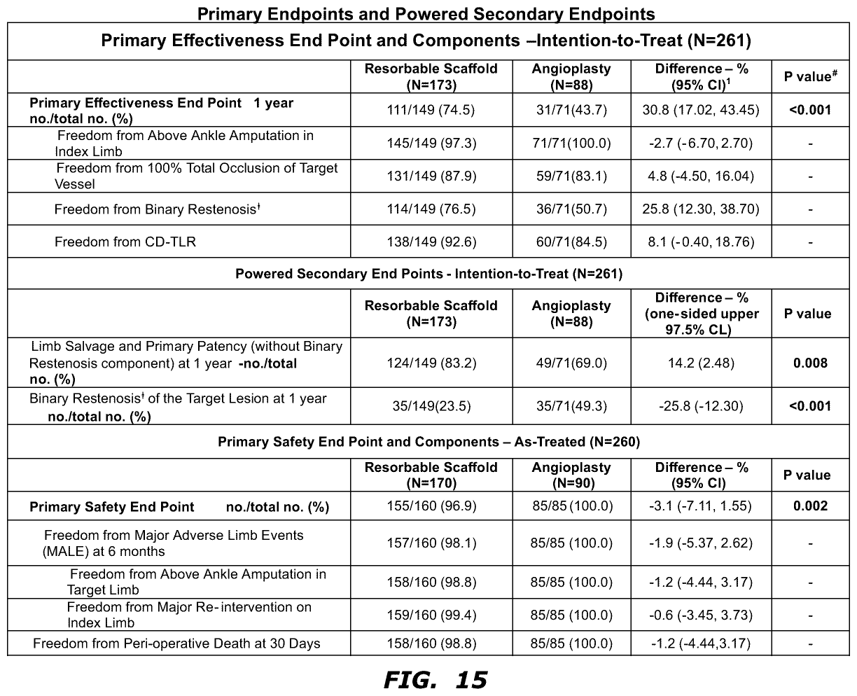

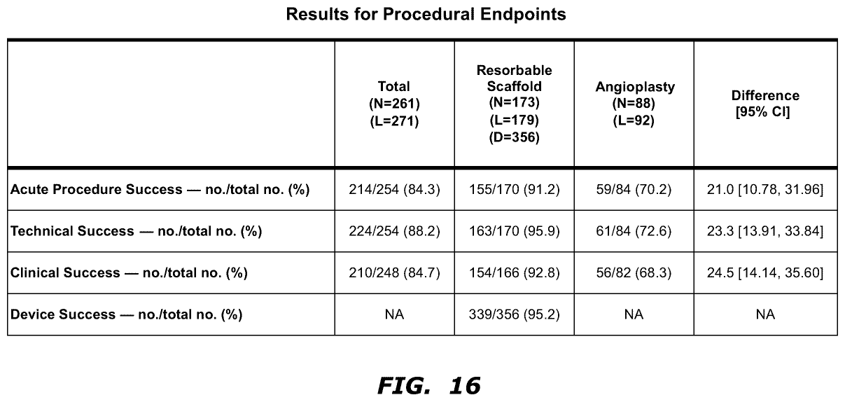

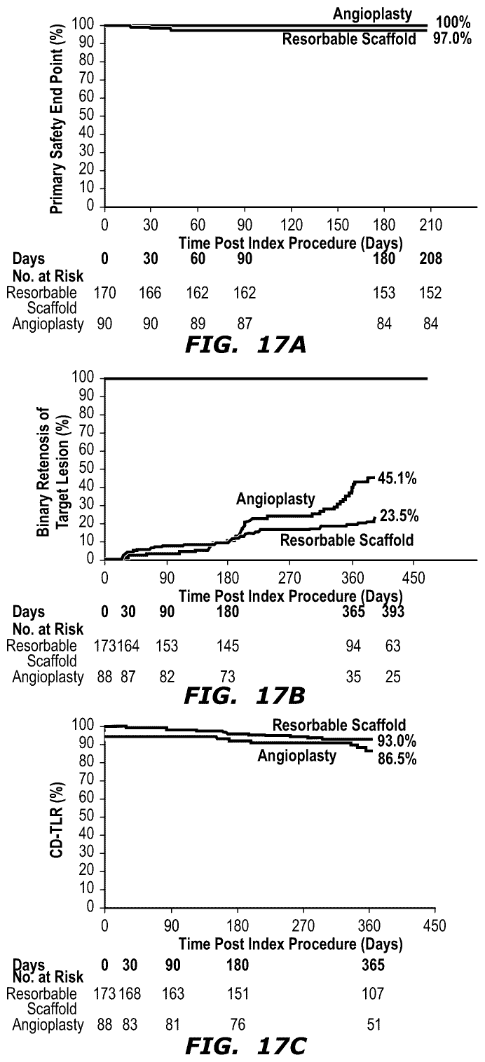

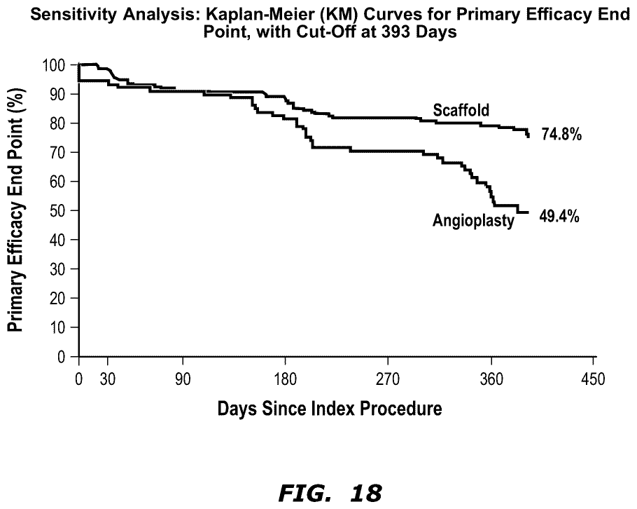

Further results of such clinical testing are shown in FIGS. 13-18.

### 2 Year Data

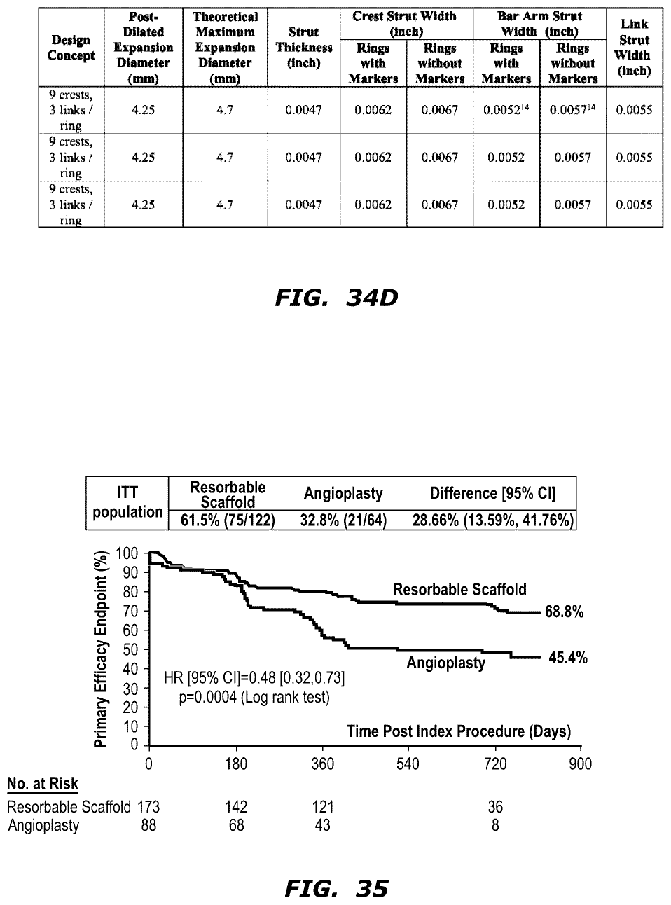

Turning to FIG. 35, the primary efficacy end point (freedom from each of: amputation above the ankle of the target limb, occlusion of the target vessel, clinically driven revascularization of the target lesion, and binary restenosis of the target lesion) was also evaluated at 2 years after insertion of the scaffold. As shown, the rates were 61.5% in the polymeric scaffold group and 32.8% in the angioplasty group of subjects, with an absolute difference of about 29 percentage points (95% confidence interval).

FIG. 36 is a landmark analysis showing limb salvage and primary patency efficacy results. It shows the event rate for 2 time periods: from 0-15 months and from 15-27 months. For a 0-27 months Kaplan-Meier analysis, if a given patient experienced multiple events during this time period, only the first event would be counted. In the illustrated landmark analysis, if a patient has one event between 0-15 months, and one event between 15-27 months, each event would be counted in its respective time period. As such, one can see that the ‘number at risk’ resets at 15 months—i.e., when a patient has an event between 0-15 months, they are censored and removed from the number at risk for this time period. But for the 15-27 month period, this patient is put back into the pool of ‘number at risk.’ As shown in FIG. 36, in terms of limb salvage and primary patency, the polymeric scaffold has higher rates for limb salvage and primary patency in both time periods, as compared to balloon angioplasty.

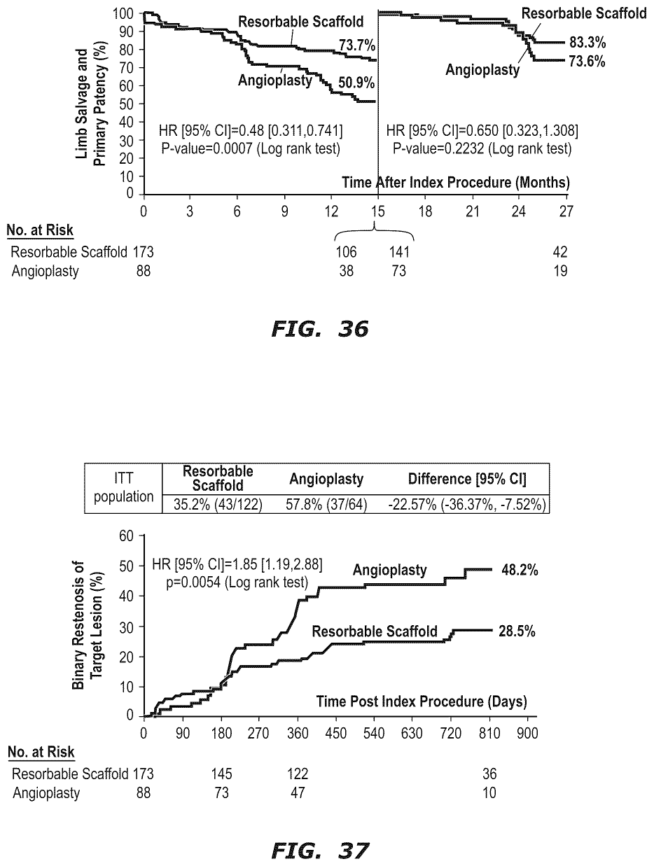

FIG. 37 shows the efficacy of the polymer scaffold, relative to balloon angioplasty, in terms of a component of the primary efficacy endpoint—binary restenosis—evaluated at 2 years after insertion of the scaffold. As shown, the rates were 35.2% for the polymeric scaffold group and 57.8% for the balloon angioplasty group, with an absolute difference of about 23 percentage points (95% confidence interval), which shows a significant advantage for the polymeric scaffold group.

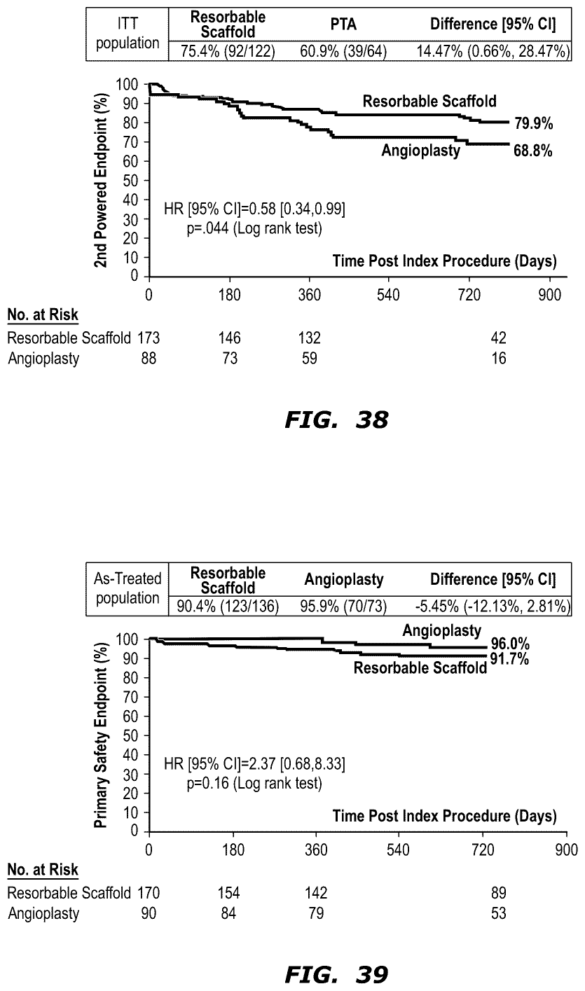

FIG. 38 shows the efficacy of the polymer scaffold, as measured by the secondary powered endpoint—freedom from the composite of above ankle amputation, total occlusion of target vessel and CD-TLR—evaluated at 2 years after insertion of the scaffold. As shown, the rates were 60.9% for the balloon angioplasty group and 75.4% for the polymeric scaffold group of subjects, with an absolute difference of about 14.5 percentage points (95% confidence interval).

These results indicate that the claimed methods also offer better long-term clinical outcomes, relative to the standard of care—balloon angioplasty.

FIG. 39 shows the safety of the polymeric scaffold, as measured by the primary safety endpoint, discussed above, evaluated at 2 years after insertion of the scaffold. As shown, the Kaplan-Meier estimate was 95.9% for the balloon angioplasty group and 90.4% for the polymeric scaffold group of subjects, with an absolute difference of about 5.5 percentage points (95% confidence interval). The results show that the claimed methods have improved effectiveness, relative to balloon angioplasty, for long-term clinical outcomes, with similar safety as compared to balloon angioplasty in the long term.

### 3 Year Data

Patients were scheduled for follow-up visits at 30 days, 3 months, 6 months, 1 year, and annually through 5 years. The 3-year follow-up visit (3 years ±28 days) included in-office assessments such as duplex ultrasound (DUS) of the target vessel, ankle-brachial index (ABI), toe-brachial index (TBI), Rutherford-Becker Class (RBC), medication review, adverse event reporting, and new wound evaluation.

When evaluating the 3 year data, the primary safety endpoint was defined as freedom from major adverse limb events (MALE) at 3 years and perioperative death (POD) at 30 days. MALE included above-ankle amputation or major reintervention (new surgical bypass grafting, interposition grafting, thrombectomy, or thrombolysis related to the target lesion). All adverse events were adjudicated by an independent, blinded clinical events committee (CEC). Imaging and wound healing assessments were evaluated by core laboratories.

The following clinical descriptive endpoints were evaluated at 3 years: amputation-free survival (defined as freedom from above-ankle amputation and death), all-cause death, improvement in RBC, arterial thrombosis, and occurrence of new wound(s) (defined as a wound below the knee in the index limb that was not identified at the time of the index procedure or a wound that had recurred in the same location following the healing of the index wound).

Of the 261 patients initially enrolled, 173 were assigned to polymeric scaffold treatment and 88 to angioplasty treatment. Although clinical data were available for 80.3% of patients treated in the polymeric scaffold group and 72.7% of those treated with angioplasty by the end of the 3-year follow-up period, some patients had to be excluded from the analysis because they did not have DUS or angiography data to evaluate patency at 3 years and they did not have other qualifying events. As a result, the analysis included data for 110 patients in the polymeric scaffold group and 60 patients in the angioplasty group.

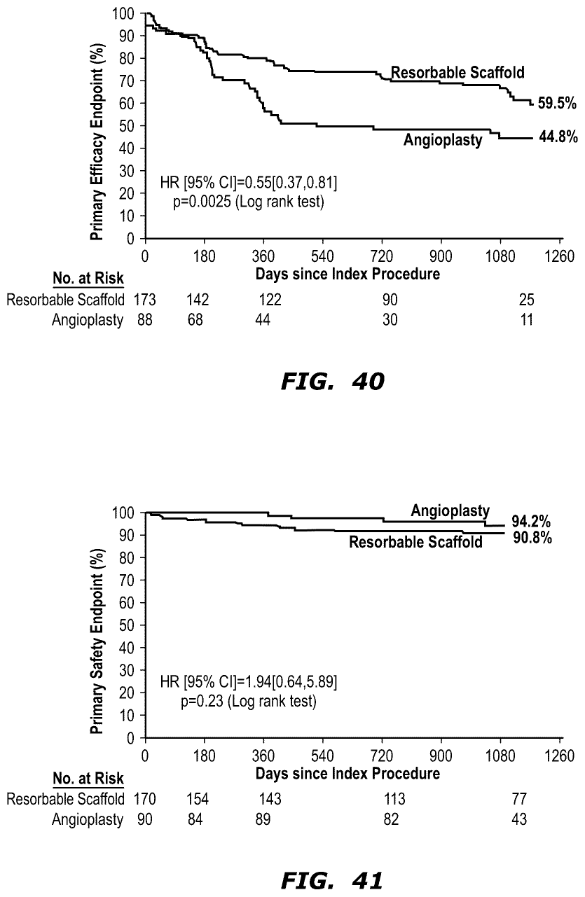

At 3 years, the primary efficacy endpoint, a composite of primary patency and limb salvage, was achieved in 50.0% in the polymeric scaffold group, compared to 26.7% in the angioplasty group corresponding to an absolute risk difference (ARD) of 23.3%. Each component of the composite endpoint followed a similar trend: freedom from binary restenosis was higher in the polymeric scaffold group (54.5% vs. 36.7%, ARD of 17.9%), and freedom from CD-TLR also favored the polymeric scaffold group (86.4% vs. 75.0%, ARD of 11.4%). Freedom from above-ankle amputation remained high in both groups (91.8% for polymeric scaffold group vs. 95.0% for angioplasty group), while freedom from 100% total occlusion was 76.4% in the polymeric scaffold group compared to 71.7% in the angioplasty group. The Kaplan-Meier curve for the primary efficacy endpoint is presented in FIG. 40. At 3 years, the primary efficacy endpoint was achieved in 59.5% of the polymeric scaffold group versus 44.8% of the angioplasty group.

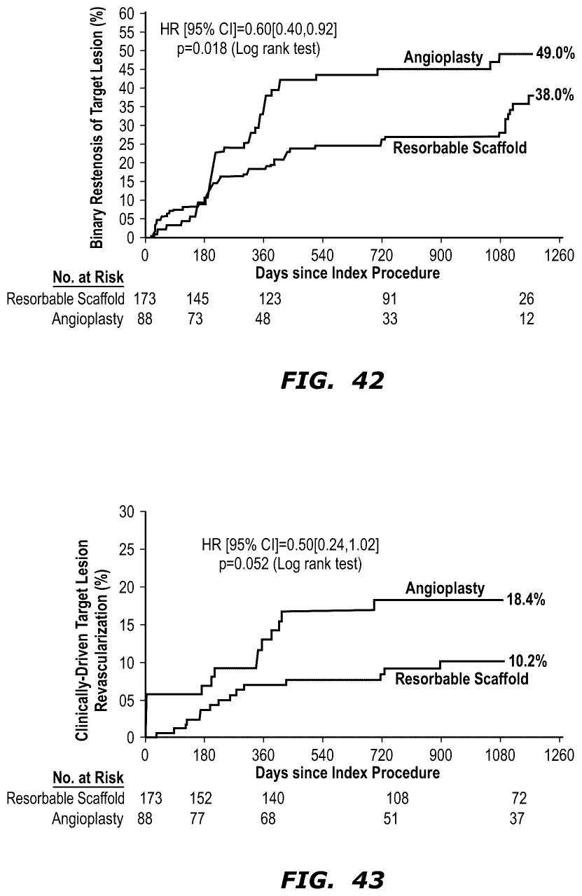

The primary safety endpoint, defined as freedom from MALE at 3 years and POD at 30 days, was evaluated in the as-treated population. The Kaplan-Meier curve for this endpoint is presented in FIG. 41. At 3 years, the primary safety endpoint was achieved in 90.8% of polymeric scaffold patients versus 94.2% of angioplasty patients.

As to binary restenosis at the 3-year follow-up, Kaplan-Meier estimates showed binary restenosis rates of 38.0% in the polymeric scaffold group and 49.0% in the angioplasty group (FIG. 42). A similar pattern was observed for CD-TLR at 3 years (FIG. 43), with lower revascularization rates in the polymeric scaffold group (10.2% vs. 18.4%).

The 3-year results demonstrate that treatment with a polymeric scaffold continues to provide improved clinical outcomes compared to angioplasty in patients with CLTI and infrapopliteal artery disease. The sustained reduction in binary restenosis and the trend toward lower CD-TLR, along with comparable safety and limb salvage rates, supports the long-term utility of polymeric scaffold treatment in this high-risk population.

Managing infrapopliteal disease in CLTI remains challenging due to the small vessel caliber, complex anatomy, and high rates of restenosis after angplasty. The trial described herein was designed to evaluate whether the use of a polymeric scaffold could overcome these limitations, e.g., particularly by combining temporary mechanical support with local everolimus delivery and subsequent resorption.

At 1 year, polymeric scaffold treatment achieved a 30.8% absolute benefit over angioplasty in the primary efficacy endpoint (composite of limb salvage and primary patency). This difference remained substantial over the long term (i.e., 28.7% at 2 years and 23.3% at 3 years), suggesting that while the margin may narrow slightly over time, the benefits advantageously persist. One possible explanation may be that the scaffold resorbs after about 3 years, and its mechanical effects diminish. In a frail population, the advantages illustrated in the data translate into fewer procedures, lower symptom burden, and better quality of life for patients suffering from peripheral artery disease, particularly infrapopliteal artery disease (e.g., chronic limb threatening ischemia (CLTI)).

In summary, the tested polymeric scaffold demonstrated a meaningful and durable advantage over angioplasty in patients with CLTI, particularly in the early post-procedure period when the risk of restenosis and limb loss is highest. The 3-year results demonstrate that the polymeric scaffold provides sustained clinical advantage over balloon angioplasty in patients with CLTI and infrapopliteal artery disease. The scaffold significantly improves primary patency, lowers rates of binary restenosis and CD-TLR, and maintains a safety profile comparable to angioplasty.

It is contemplated that various combinations or sub-combinations of the specific features and aspects of the embodiments disclosed above may be made and still fall within one or more of the embodiments. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed embodiments. Thus, it is intended that the scope of the present disclosure herein disclosed should not be limited by the particular disclosed embodiments described above. Moreover, while the present disclosure is susceptible to various modifications, and alternative forms, specific examples thereof have been shown in the drawings and are herein described in detail. It should be understood, however, that the present disclosure is not to be limited to the particular forms or methods disclosed, but to the contrary, the present disclosure is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, and “substantially” as used herein include the recited numbers (e.g., about 10%=10%), and also represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

For purposes of the present disclosure and appended claims, the conjunction “or” is to be construed inclusively (e.g., “an apple or an orange” would be interpreted as “an apple, or an orange, or both”; e.g., “an apple, an orange, or an avocado” would be interpreted as “an apple, or an orange, or an avocado, or any two, or all three”), unless: (i) it is explicitly stated otherwise, e.g., by use of “either . . . or,” “only one of,” or similar language; or (ii) two or more of the listed alternatives are mutually exclusive within the particular context, in which case “or” would encompass only those combinations involving non-mutually-exclusive alternatives. For purposes of the present disclosure and appended claims, the words “comprising,” “including,” “having,” and variants thereof, wherever they appear, shall be construed as open-ended terminology, with the same meaning as if the phrase “at least”were appended after each instance thereof.

Following are some further example embodiments of the invention. These are presented only by way of example and are not intended to limit the scope of the invention in any way. Further, any example embodiment can be combined with one or more of the example embodiments.

Embodiment 1. A method for treating peripheral artery disease (PAD) below the knee, comprising:
inserting into a peripheral artery below the knee at the site of a target lesion of a subject a scaffold made from a material comprising a polymer, wherein the scaffold may be configured for being crimped to a balloon,
the scaffold having a pattern of interconnected elements, the interconnected elements comprising struts and links, and
performing duplex ultrasound (DUS) and/or angiography after inserting the scaffold into the peripheral artery below the knee to measure binary restenosis at the site of the target lesion,
wherein the binary restenosis is defined as the presence of a hemodynamically significant restenosis of >50% identified through angiography, or peak systolic velocity ratio (PSVR) ≥2.0 as identified by DUS.

Embodiment 2. The method of embodiment 1 or other described embodiment, wherein the DUS and/or angiography is performed at one or more of 30 days (+/−14 days), 3 months (+/−14 days), 6 months (+/−28 days), 1 year (+/−28 days), 2 years (+/−28 days), 3 years (+/−28 days), 4 years (+/−28 days), or 5 years (+/−28 days) after inserting the scaffold into the peripheral artery below the knee.

Embodiment 3. The embodiment of claim 1 or other described embodiment, further comprising, when the DUS indicates the presence of an abnormal reference peak systolic velocity (PSV), identifying one or more correlating factors, the correlating factors comprising one or more of:
(1) a focal increase in the absolute peak systolic velocity (PSV) at the site of the target lesion,
(2) a spectral broadening of the waveform at the site of the target lesion,
(3) a post-stenotic turbulence (PST) and/or a change in a waveform shape and/or drop in peak systolic velocity distal to the site of the target lesion, or
(4) a review of one or more B-mode images showing a plaque burden at the site of the target lesion.

Embodiment 4. The method of embodiment 1 or other described embodiment, wherein the subject is selected based on the subject having at least a 70% stenosis at the site of the target lesion prior to insertion of the scaffold.

Embodiment 5. The method of embodiment 1 or other described embodiment, wherein the scaffold is coated with a coating comprised of an active pharmaceutical ingredient and bioresorbable poly (D,L-lactide) (PDLLA).

Embodiment 6. The method of embodiment 5 or other described embodiment, wherein the active pharmaceutical ingredient comprises everolimus, sirolimus, or other Limus drug.

Embodiment 7. The method of embodiment 1 or other described embodiment, wherein the polymer comprises at least one of poly(L-lactide) (“PLLA”), poly(L-lactide-coglycolide) (“PLGA”), polyD-lactide-co-glycolide) or poly (L-lactide-co-D-lactide) (“PLLA-co-PDLA”) with less than 10% D-lactide, or PLLD/PDLA stereo complex.

Embodiment 8. The method of embodiment 1 or other described embodiment, wherein the polymer is bioresorbable.

Embodiment 9.The method of embodiment 1 or other described embodiment, wherein the struts and the links each have a width and a thickness, and wherein an aspect ratio (AR) of the width to thickness of at least some of the struts or at least some of the links is from about 0.8 to about 1.4.

Embodiment 10. The method of embodiment 1 or other described embodiment, wherein the links each have a width and a thickness, and wherein an aspect ratio (AR) of the width to thickness of at least some of the links is from about 0.4 to about 0.9.

Embodiment 11. The method of embodiment 1 or other described embodiment, wherein the angiography and/or the DUS indicates a final residual stenosis of <30% at at least one of 30 days (+/−14 days), 3 months (+/−14 days), 6 months (+/−28 days), 1 year (+/−28 days), 2 years (+/−28 days), 3 years (+/−28 days), 4 years (+/−28 days), or 5 years (+/−28 days) after inserting the scaffold into the peripheral artery below the knee.

Embodiment 12. The method of embodiment 1 or other described embodiment, further comprising:
when the angiography and/or the DUS indicates that there is a binary restenosis, performing an interventional procedure, the interventional procedure being selected from angioplasty, implanting of a second resorbable scaffold or a metallic stent, brachytherapy, atherectomy, thrombectomy, intravascular lithotripsy, remedial surgery, genetic treatment with gene transfer or stem cell infusion.

Embodiment 13. The method of embodiment 1 or other described embodiment, wherein if both angiography and DUS are performed, the angiography takes precedence over the DUS for the determination of the presence of binary restenosis.

Embodiment 14. The method of embodiment 1 or other described embodiment, wherein the scaffold exhibits a crush recoverability of at least about 90% after a 50% crushing load.

Embodiment 15. The method of embodiment 1 or other described embodiment, wherein the scaffold exhibits a crush recoverability of at least about 95% after a 50% crushing load.

Embodiment 16. The method of embodiment 1 or other described embodiment, wherein the scaffold exhibits a crush recoverability of at least about 90% after a 75% crushing load.

Embodiment 17. The method of embodiment 1 or other described embodiment, wherein the scaffold exhibits a pinching stiffness of at least about 0.5 N/mm, a radial strength of at least about 0.3 N/mm, and/or a wall thickness of at least about 0.008″, or from about 0.008″ to about 0.014″.

Embodiment 18. The method of embodiment 1 or other described embodiment, wherein the peripheral artery is an infrapopliteal artery.

Embodiment 19. The method of embodiment 1 or other described embodiment, wherein the subject is known to have symptomatic chronic limb threatening ischemia (CLTI) prior to insertion of the scaffold.

Embodiment 20. The method of embodiment 1 or other described embodiment, wherein insertion of the scaffold reduces the risk, relative to balloon angioplasty, of each of (1) a total occlusion of the site of the target lesion, (2) a binary restenosis of the site of the target lesion, and (3) a clinically driven target lesion revascularization (CD-TLR) for at least 1 year after insertion of the scaffold.

Embodiment 21. The method of embodiment 1 or other described embodiment, wherein after 1 year after inserting the scaffold into the peripheral artery, the subject exhibits enhanced freedom from the following, relative to balloon angioplasty:
(1) a total occlusion of the site of the target lesion,
(2) binary restenosis of the site of the target lesion, and
(3) a clinically driven target lesion revascularization (CD-TLR).

Embodiment 22. The method of embodiment 1 or other described embodiment, wherein insertion of the scaffold is non-inferior to balloon angioplasty for preventing:
(1) a major adverse limb event (MALE), wherein MALE is defined as an above-ankle amputation and/or re-intervention, including thrombosis, at 6 months after inserting the scaffold into the peripheral artery below the knee, and
(2) death of the subject within 30 days of inserting the scaffold into the peripheral artery below the knee.

Embodiment 23. The method of embodiment 1 or other described embodiment, wherein each ring includes struts and crowns, wherein the struts are configured to fold at the crowns when the stent is crimped to the balloon.

Embodiment 24. The method of embodiment 23 or other described embodiment, wherein each of the rings forms includes U crowns, and the links connected to their respective rings form, with the respective rings, either a W crown or a Y crown.

Embodiment 25. The method of embodiment 1 or other described embodiment, wherein insertion of the scaffold is effective to reduce the rate of restenosis below the knee to no more than 50% for at least 1 year after insertion of the scaffold into the peripheral artery.

Embodiment 26. The method of embodiment 1 or other described embodiment, wherein insertion of the scaffold is effective to achieve an improvement of at least 13%, at least 15%, at least 20%, at least 25%, at least 30%, from 13% to 50%, 13% to 45%, 13% to 40% or from 13% to 35%, compared to balloon angioplasty, in treatment of the target lesion, based on one or more, or a composite of the following criteria: freedom from (1) an above ankle amputation, (2) a total occlusion of the site of the target lesion, (3) a binary restenosis of the site of the target lesion, and (4) a clinically driven target lesion revascularization (CD-TLR) for at least 1 year after insertion of the scaffold.

Embodiment 27. The method of embodiment 1 or other described embodiment, wherein insertion of the scaffold is effective to achieve an improvement of at least 11%, at least 15%, at least 20%, or at least 25%, compared to balloon angioplasty, in treatment of the target lesion, for at least 1 year after insertion of the scaffold, based on a binary restenosis of the site of the target lesion.

Embodiment 28. The method of embodiment 1 or other described embodiment, wherein insertion of the scaffold improves CLTI in the subject to a greater extent than a balloon angioplasty standard of care, the improvement being measured by limb salvage and vessel patency, for at least 1 year after insertion of the scaffold.

Embodiment 29. The method of embodiment 1 or other described embodiment, wherein insertion of the scaffold is effective to improve the outcome of treatment in the subject compared to a balloon angioplasty standard of care, for at least 1 year after insertion of the scaffold, wherein the outcome of treatment is measured by limb salvage and vessel patency.

Embodiment 30. The method of embodiment 1 or other described embodiment, wherein insertion of the scaffold is superior to a balloon angioplasty standard of care, as measured by limb salvage and vessel patency, for at least 1 year after insertion of the scaffold.

Embodiment 31. The method of embodiment 1 or other described embodiment, wherein insertion of the scaffold is effective to, relative to balloon angioplasty, reduce the occurrence of target lesion revascularization for at least 1 year after insertion of the scaffold.

Embodiment 32. The method of embodiment 1 or other described embodiment, wherein insertion of the scaffold is effective to reduce a target lesion revascularization rate in the subject compared to a balloon angioplasty standard of care, for at least 1 year after insertion of the scaffold.

Embodiment 33. The method of embodiment 1 or other described embodiment, wherein insertion of the scaffold is as safe for the subject as a balloon angioplasty standard of care method, wherein safe is defined as freedom from both of the following:
(1) a major adverse limb event (MALE), wherein MALE is defined as an above-ankle amputation and/or re-intervention, including thrombosis, at 6 months after inserting the scaffold into the peripheral artery below the knee, and
(2) death of the subject within 30 days of inserting the scaffold into the peripheral artery below the knee.

Embodiment 34. The method of embodiment 1 or other described embodiment, wherein insertion of the scaffold is effective to reduce the rate of restenosis below the knee to no more than 50% for at least 1 year after insertion of the scaffold into the peripheral artery and is non-inferior to balloon angioplasty in preventing:
(1) a major adverse limb event (MALE), wherein MALE is defined as an above-ankle amputation and/or re-intervention, including thrombosis, at 6 months after inserting the scaffold into the peripheral artery below the knee, and
(2) death of the subject within 30 days of inserting the scaffold into the peripheral artery below the knee.

Embodiment 35. The method of embodiment 1 or other described embodiment, wherein the scaffold reduces acute vessel recoil to no more than 30%.

Embodiment 36. A method for treating peripheral artery disease (PAD) below the knee, comprising:
inserting into a peripheral artery below the knee at the site of a target lesion of a subject a scaffold made from a material comprising a polymer, the scaffold having a pre-crimp diameter and a wall thickness such that a ratio of the pre-crimp diameter to the wall thickness is from about 30 to about 60, wherein the scaffold may be configured for being crimped to a balloon,
the scaffold having a pattern of interconnected elements, the interconnected elements comprising struts and links, and
performing duplex ultrasound (DUS) and/or angiography after inserting the scaffold into the peripheral artery below the knee to measure binary restenosis at the site of the target lesion,
wherein the binary restenosis is defined as the presence of a hemodynamically significant restenosis of >50% identified through angiography, or peak systolic velocity ratio (PSVR) ≥2.0 as identified by DUS.

Embodiment 37. A method for treating peripheral artery disease (PAD) below the knee, comprising:
inserting into a peripheral artery below the knee at the site of a target lesion of a subject a scaffold made from a material comprising a polymer, wherein the scaffold may be configured for being crimped to a balloon,
the scaffold having a pattern of interconnected elements, the interconnected elements comprising struts and links, and
performing duplex ultrasound (DUS) and/or angiography after inserting the scaffold into the peripheral artery below the knee to measure binary restenosis at the site of the target lesion,
wherein the binary restenosis is defined as the presence of a hemodynamically significant restenosis of >50% identified through angiography, or peak systolic velocity ratio (PSVR) ≥2.0 as identified by DUS, and
wherein when the DUS indicates the presence of an abnormal reference peak systolic velocity (PSV), the method further comprising identifying one or more correlating factors, the correlating factors comprising one or more of:
   (1) a focal increase in the absolute peak systolic velocity (PSV) at the site of the target lesion,
   (2) a spectral broadening of the waveform at the site of the target lesion,
   (3) a post-stenotic turbulence (PST) and/or a change in a waveform shape and/or drop in peak systolic velocity distal to the site of the target lesion, or
   (4) a review of one or more B-mode images showing a plaque burden at the site of the target lesion, and
based on the identification of the one or more correlating factors, performing further interventional steps based on the confirmation of binary restenosis.

Embodiment 38. A method for treating peripheral artery disease (PAD) below the knee, comprising:
inserting into a peripheral artery below the knee at the site of a target lesion of a subject a scaffold made from a material comprising a polymer, wherein the scaffold may be configured for being crimped to a balloon,
the scaffold having a pattern of interconnected elements, the interconnected elements comprising struts and links, and
performing duplex ultrasound (DUS) and/or angiography after inserting the scaffold into the peripheral artery below the knee to measure binary restenosis at the site of the target lesion,
wherein the binary restenosis is defined as the presence of a hemodynamically significant restenosis of >50% identified through angiography, or peak systolic velocity ratio (PSVR) ≥2.0 as identified by DUS,
wherein the DUS and/or angiography is performed at one or more of 30 days (+/−14 days), 3 months (+/−14 days), 6 months (+/−28 days), 1 year (+/−28 days), 2 years (+/−28 days), 3 years (+/−28 days), 4 years (+/−28 days), or 5 years (+/−28 days) after inserting the scaffold into the peripheral artery below the knee, and
wherein if the angiography and/or the DUS indicates that there is binary restenosis, the method further comprising performing an interventional procedure, the interventional procedure being selected from angioplasty, implanting of a second resorbable scaffold or a metallic stent, brachytherapy, atherectomy, thrombectomy, intravascular lithotripsy, remedial surgery, genetic treatment with gene transfer or stem cell infusion.

Embodiment 39. A method for treating peripheral artery disease (PAD) below the knee, comprising:
inserting into a peripheral artery below the knee at the site of a target lesion of a subject a scaffold made from a material comprising a polymer, wherein the scaffold may be configured for being crimped to a balloon,
the scaffold having a pattern of interconnected elements, the interconnected elements comprising struts and links, and
performing duplex ultrasound (DUS) and/or angiography after inserting the scaffold into the peripheral artery below the knee to measure binary restenosis at the site of the target lesion,
wherein the binary restenosis is defined as the presence of a hemodynamically significant restenosis of >50% identified through angiography, or peak systolic velocity ratio (PSVR) ≥2.0 as identified by DUS,
wherein the DUS and/or angiography is performed at one or more of 30 days (+/−14 days), 3 months (+/−14 days), 6 months (+/−28 days), 1 year (+/−28 days), 2 years (+/−28 days), 3 years (+/−28 days), 4 years (+/−28 days), or 5 years (+/−28 days) after inserting the scaffold into the peripheral artery below the knee,
wherein when the DUS indicates the presence of an abnormal reference peak systolic velocity (PSV), the method further comprising identifying one or more correlating factors, the correlating factors comprising one or more of:
   (1) a focal increase in the absolute peak systolic velocity (PSV) at the site of the target lesion,
   (2) a spectral broadening of the waveform at the site of the target lesion,
   (3) a post-stenotic turbulence (PST) and/or a change in a waveform shape and/or drop in peak systolic velocity distal to the site of the target lesion, or
   (4) a review of one or more B-mode images showing a plaque burden at the site of the target lesion, and
based on the identification of the one or more correlating factors, performing further interventional steps due to the confirmation of binary restenosis.

Embodiment 40. A method for treating peripheral artery disease (PAD) below the knee, comprising:
inserting into a peripheral artery below the knee at the site of a target lesion of a subject a scaffold made from a material comprising a polymer, wherein the scaffold may be configured for being crimped to a balloon, wherein the polymer is bioresorbable,
the scaffold having a pattern of interconnected elements, the interconnected elements comprising struts and links,
wherein the scaffold exhibits a crush recoverability of at least about 90% after a 50% crushing load,
further wherein the scaffold exhibits at least one of a pinching stiffness of at least about 0.5 N/mm, a radial strength of at least about 0.3 N/mm, a wall thickness of at least about 0.008″, or a wall thickness from about 0.008″ to about 0.014″, and
performing duplex ultrasound (DUS) and/or angiography after inserting the scaffold into the peripheral artery below the knee to measure binary restenosis at the site of the target lesion,
wherein the binary restenosis is defined as the presence of a hemodynamically significant restenosis of >50% identified through angiography, or peak systolic velocity ratio (PSVR) ≥2.0 as identified by DUS.

Embodiment 41. A method for treating peripheral artery disease (PAD) below the knee, comprising:
inserting into a peripheral artery below the knee at the site of a target lesion in a target vessel of a subject a scaffold made from a material comprising a polymer, wherein the scaffold may be configured for being crimped to a balloon,
the scaffold having a pattern of interconnected elements, the interconnected elements comprising struts and links, and
performing duplex ultrasound (DUS) and/or angiography after inserting the scaffold into the peripheral artery below the knee to measure binary restenosis at the site of the target lesion,
wherein the binary restenosis is defined as the presence of a hemodynamically significant restenosis of >50% identified through angiography, or peak systolic velocity ratio (PSVR) ≥2.0 as identified by DUS, and
wherein insertion of the scaffold is effective to, relative to balloon angioplasty, reduce the risk of the following for at least one year after insertion of the scaffold:
   (1) a total occlusion of the target vessel,
   (2) a binary restenosis of the target lesion, and
   (3) a clinically driven target lesion revascularization (CD-TLR).

Embodiment 42. A method for treating peripheral artery disease (PAD) below the knee, comprising:
inserting into a peripheral artery below the knee at the site of a target lesion of a subject a scaffold made from a material comprising a polymer, wherein the scaffold may be configured for being crimped to a balloon,
the scaffold having a pattern of interconnected elements, the interconnected elements comprising struts and links, and
performing duplex ultrasound (DUS) and/or angiography after inserting the scaffold into the peripheral artery below the knee to measure binary restenosis at the site of the target lesion,
wherein the binary restenosis is defined as the presence of a hemodynamically significant restenosis of >50% identified through angiography, or peak systolic velocity ratio (PSVR) ≥2.0 as identified by DUS, and
wherein after 1 year after inserting the scaffold into the peripheral artery, the subject exhibits, relative to balloon angioplasty, a non-inferior rate of freedom from both of the following:
   (1) a major adverse limb event (MALE), wherein MALE is defined as an above-ankle amputation and/or re-intervention, including thrombosis, at 6 months after inserting the scaffold into the peripheral artery below the knee, and
   (2) death within 30 days of inserting the scaffold into the peripheral artery below the knee.

Embodiment 43. A method for treating peripheral artery disease (PAD) below the knee, comprising:
inserting into a peripheral artery below the knee at the site of a target lesion of a subject a scaffold made from a material comprising a polymer, wherein the scaffold may be configured for being crimped to a balloon, the scaffold having a pattern of interconnected elements, the interconnected elements comprising struts and links, the scaffold being configured to, after 1 year after inserting the scaffold into the peripheral artery, reduce, relative to balloon angioplasty, the risk of the following in the subject: (1) a total occlusion of the site of the target lesion, (2) a binary restenosis of the site of the target lesion, and (3) a clinically driven target lesion revascularization (CD-TLR); and
periodically performing duplex ultrasound (DUS) and/or angiography after inserting the scaffold into the peripheral artery below the knee to measure binary restenosis at the site of the target lesion,
wherein the binary restenosis is defined as the presence of the restenosis being a hemodynamically significant restenosis of >50% identified through angiography, or a peak systolic velocity ratio (PSVR) ≥2.0 as identified by DUS.

Embodiment 44. A method for treating peripheral artery disease (PAD) below the knee, comprising:
inserting into a peripheral artery below the knee at the site of a target lesion of a target vessel of a subject a scaffold made from a material comprising a polymer, wherein the scaffold may be configured for being crimped to a balloon, the scaffold having a pattern of interconnected elements, the interconnected elements comprising struts and links, the scaffold being configured to, within 1 year of inserting the scaffold into the peripheral artery, reduce, relative to balloon angioplasty, the risk of all of the following in the patient: (1) a total occlusion of the target vessel, (2) a binary restenosis of the target lesion, and (3) a clinically driven target lesion revascularization (CD-TLR); and
periodically performing duplex ultrasound (DUS) after inserting the scaffold into the peripheral artery below the knee to measure binary restenosis at the site of the target lesion,
wherein the binary restenosis is defined as the presence of the restenosis being a hemodynamically significant restenosis of >50% identified through angiography, or a peak systolic velocity ratio (PSVR) ≥2.0 as identified by DUS.

Embodiment 45. The method of embodiment 44 or other described embodiment, wherein insertion of the scaffold is effective to achieve an improvement of at least 3%, compared to balloon angioplasty in treatment of the target lesion, based on the following criteria: freedom from a total occlusion of the target vessel for at least 1 year after insertion of the scaffold.

Embodiment 46. The method of embodiment 44 or other described embodiment, wherein insertion of the scaffold is effective to achieve an improvement of at least 3%, 5%, or 7%, compared to balloon angioplasty in treatment of the target lesion, based on the following criteria: freedom from a clinically driven target lesion revascularization (CD-TLR) for at least 1 year after insertion of the scaffold.

Embodiment 47. The method of embodiment 44 or other described embodiment, wherein insertion of the scaffold is effective to achieve an improvement of at least 15%, or at least 20% compared to balloon angioplasty in the treatment of the target lesion, based on binary restenosis, for at least 1 year after insertion of the scaffold.

Embodiment 48. The method of embodiment 44 or other described embodiment, wherein insertion of the scaffold is effective to achieve an improvement of at least 15%, at least 20% or at least 25%, compared to balloon angioplasty, for at least 2 years after insertion of the scaffold, in the treatment of the target lesion, based on a composite of the following criteria: freedom from (1) an above ankle amputation, (2) a total occlusion of the target vessel, (3) a binary restenosis of the target lesion, and (4) a clinically driven target lesion revascularization (CD-TLR).

Embodiment 49. The method of embodiment 44 or other described embodiment, wherein insertion of the scaffold is effective to achieve an improvement of at least 15%, at least 20%, or at least 25%, compared to balloon angioplasty, for at least 2 years after insertion of the scaffold, in the treatment of the target lesion, based on limb salvage and primary patency.

Embodiment 50. The method of embodiment 44 or other described embodiment, wherein insertion of the scaffold is effective to achieve an improvement of at least 15%, or at least 20%, compared to balloon angioplasty, for at least 2 years after insertion of the scaffold, in the treatment of the target lesion, based on binary restenosis.

Embodiment 51. The method of embodiment 44 or other described embodiment, wherein insertion of the scaffold is effective to, relative to balloon angioplasty, reduce the risk of the following for at least 2 years after insertion of the scaffold:
(1) a total occlusion of the target vessel,
(2) a binary restenosis of the target lesion, and
(3) a clinically driven target lesion revascularization (CD-TLR).

Embodiment 52. The method of embodiment 44 or other described embodiment, wherein insertion of the scaffold is non-inferior in terms of safety for the subject, relative to balloon angioplasty, evaluated at 2 years after insertion of the scaffold wherein safety is defined as freedom from both of the following:
(1) a major adverse limb event (MALE), wherein MALE is defined as an above-ankle amputation and/or re-intervention, and
(2) death of the subject within 30 days of inserting the scaffold into the peripheral artery below the knee.

Embodiment 53. The method of embodiment 44 or other described embodiment, wherein the difference in safety, relative to balloon angioplasty, evaluated at 2 years after insertion of the scaffold and based on the p-value, is insignificant, wherein safety is defined as freedom from both of the following:
(1) a major adverse limb event (MALE), wherein MALE is defined as an above-ankle amputation and/or re-intervention, and
(2) death of the subject within 30 days of inserting the scaffold into the peripheral artery below the knee.

Embodiment 54. The method of embodiment 44 or other described embodiment, wherein insertion of the scaffold is effective to achieve an improvement of at least 5%, at least 10%, at least 15% or at least 20%, compared to balloon angioplasty, for at least 3 years after insertion of the scaffold, in the treatment of the target lesion, based on a composite of the following criteria: freedom from (1) an above ankle amputation, (2) a total occlusion of the target vessel, (3) a binary restenosis of the target lesion, and (4) a clinically driven target lesion revascularization (CD-TLR).

Embodiment 55. The method of embodiment 44 or other described embodiment, wherein insertion of the scaffold is effective to achieve an improvement of at least 5%, at least 10%, at least 15%, or at least 20%, compared to balloon angioplasty, for at least 3 years after insertion of the scaffold, in the treatment of the target lesion, based on limb salvage and primary patency.

Embodiment 56. The method of embodiment 44 or other described embodiment, wherein insertion of the scaffold is effective to achieve an improvement of at least 5%, or at least 10%, compared to balloon angioplasty, for at least 3 years after insertion of the scaffold, in the treatment of the target lesion, based on binary restenosis.

Embodiment 57. The method of embodiment 44 or other described embodiment, wherein insertion of the scaffold is effective to, relative to balloon angioplasty, reduce the risk of the following for at least 3 years after insertion of the scaffold:
(1) a total occlusion of the target vessel,
(2) a binary restenosis of the target lesion, and
(3) a clinically driven target lesion revascularization (CD-TLR).

Embodiment 58. The method of embodiment 44 or other described embodiment, wherein insertion of the scaffold is non-inferior in terms of safety for the subject, relative to balloon angioplasty, evaluated at 3 years after insertion of the scaffold wherein safety is defined as freedom from both of the following:
(1) a major adverse limb event (MALE), wherein MALE is defined as an above-ankle amputation and/or re-intervention, and
(2) death of the subject within 30 days of inserting the scaffold into the peripheral artery below the knee.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

1. A method for treating peripheral artery disease (PAD) below the knee, comprising:
inserting into a peripheral artery below a knee at a site of a target lesion of a subject a scaffold made from a material comprising a polymer,
the scaffold having a pattern of interconnected elements, the interconnected elements comprising struts and links, and
performing duplex ultrasound (DUS) and/or angiography after inserting the scaffold into the peripheral artery below the knee to measure binary restenosis at the site of the target lesion,
wherein the binary restenosis is defined as the presence of a hemodynamically significant restenosis of >50% identified through angiography, or peak systolic velocity ratio (PSVR) ≥2.0 as identified by DUS.

2. The method of claim 1, wherein the scaffold is configured for being crimped to a balloon.

3. The method of claim 1, wherein the DUS and/or angiography is performed at one or more of 30 days (+/−14 days), 3 months (+/−14 days), 6 months (+/−28 days), 1 year (+/−28 days), 2 years (+/−28 days), 3 years (+/−28 days), 4 years (+/−28 days), or 5 years (+/−28 days) after inserting the scaffold into the peripheral artery below the knee.

4. The method of claim 1, further comprising, when the DUS indicates the presence of an abnormal reference peak systolic velocity (PSV), identifying one or more correlating factors, the correlating factors comprising one or more of:
(1) a focal increase in the absolute peak systolic velocity (PSV) at the site of the target lesion,
(2) a spectral broadening of the waveform at the site of the target lesion,
(3) a post-stenotic turbulence (PST) and/or a change in a waveform shape and/or drop in peak systolic velocity distal to the site of the target lesion, or
(4) a review of one or more B-mode images showing a plaque burden at the site of the target lesion.

5. The method of claim 1, wherein the subject is selected based on the subject having at least a 70% stenosis at the site of the target lesion prior to insertion of the scaffold.

6. The method of claim 1, wherein the scaffold is coated with a coating comprised of an active pharmaceutical ingredient and bioresorbable poly (D,L-lactide) (PDLLA), wherein the active pharmaceutical ingredient comprises everolimus, sirolimus, or other Limus drug.

7. (canceled)

8. The method of claim 1, wherein the polymer comprises at least one of poly(L-lactide) (“PLLA”), poly(L-lactide-coglycolide) (“PLGA”), polyD-lactide-co-glycolide) or poly (L-lactide-co-D-lactide) (“PLLA-co-PDLA”) with less than 10% D-lactide, or PLLD/PDLA stereo complex.

9. The method of claim 1, wherein the polymer is bioresorbable.

10. (canceled)

11. (canceled)

12. (canceled)

13. (canceled)

14. The method of claim 1, wherein if both angiography and DUS are performed, the angiography takes precedence over the DUS for the determination of the presence of binary restenosis.

15. (canceled)

16. (canceled)

17. (canceled)

18. (canceled)

19. The method of claim 1, wherein the peripheral artery is an infrapopliteal artery, and the subject is known to have symptomatic chronic limb threatening ischemia (CLTI) prior to insertion of the scaffold.

20. (canceled)

21. (canceled)

22. The method of claim 1, wherein after 1 year after inserting the scaffold into the peripheral artery, the subject exhibits enhanced freedom from the following, relative to balloon angioplasty:
(1) a total occlusion of the site of the target lesion,
(2) binary restenosis of the site of the target lesion, and
(3) a clinically driven target lesion revascularization (CD-TLR).

23. (canceled)

24. (canceled)

25. (canceled)

26. (canceled)

27. The method of claim 1, wherein insertion of the scaffold is effective to achieve an improvement of at least 13%, at least 15%, at least 20%, at least 25%, at least 30%, from 13% to 50%, 13% to 45%, 13% to 40% or from 13% to 35%, compared to balloon angioplasty, in treatment of the target lesion, based on one or more, or a composite of the following criteria: freedom from (1) an above ankle amputation, (2) a total occlusion of the site of the target lesion, (3) a binary restenosis of the site of the target lesion, and (4) a clinically driven target lesion revascularization (CD-TLR) for at least 1 year after insertion of the scaffold.

28. The method of claim 1, wherein insertion of the scaffold is effective to achieve an improvement of at least 11%, at least 15%, at least 20%, or at least 25%, compared to balloon angioplasty, in treatment of the site of the target lesion, for at least 1 year after insertion of the scaffold, based on a binary restenosis of the site of the target lesion.

29. (canceled)

30. (canceled)

31. The method of claim 1, wherein insertion of the scaffold is superior to a balloon angioplasty standard of care, as measured by limb salvage and vessel patency, for at least 1 year after insertion of the scaffold.

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. A method for treating peripheral artery disease (PAD) below the knee, comprising:
inserting into a peripheral artery below the knee at the site of a target lesion of a target vessel of a subject a scaffold made from a material comprising a polymer, the scaffold having a pattern of interconnected elements, the interconnected elements comprising struts and links, the scaffold being configured to, within 1 year of inserting the scaffold into the peripheral artery, reduce, relative to balloon angioplasty, the risk of all of the following in the patient: (1) a total occlusion of the target vessel, (2) a binary restenosis of the target lesion, and (3) a clinically driven target lesion revascularization (CD-TLR); and
periodically performing duplex ultrasound (DUS) after inserting the scaffold into the peripheral artery below the knee to measure binary restenosis at the site of the target lesion, wherein the binary restenosis is defined as the presence of the restenosis being a hemodynamically significant restenosis of >50% identified through angiography, or a peak systolic velocity ratio (PSVR) ≥2.0 as identified by DUS.

54. (canceled)

55. (canceled)

56. (canceled)

57. (canceled)

58. (canceled)

59. The method of claim 53, wherein insertion of the scaffold is effective to achieve an improvement of at least 15%, at least 20%, or at least 25%, compared to balloon angioplasty, for at least 2 years after insertion of the scaffold, in the treatment of the target lesion, based on limb salvage and primary patency.

60. (canceled)

61. (canceled)

62. (canceled)

63. (canceled)

64. The method of claim 53, wherein insertion of the scaffold is effective to achieve an improvement of at least 5%, at least 10%, at least 15% or at least 20%, compared to balloon angioplasty, for at least 3 years after insertion of the scaffold, in the treatment of the target lesion, based on a composite of the following criteria: freedom from (1) an above ankle amputation, (2) a total occlusion of the target vessel, (3) a binary restenosis of the target lesion, and (4) a clinically driven target lesion revascularization (CD-TLR).

65. The method of claim 53, wherein insertion of the scaffold is effective to achieve an improvement of at least 5%, at least 10%, at least 15%, or at least 20%, compared to balloon angioplasty, for at least 3 years after insertion of the scaffold, in the treatment of the target lesion, based on limb salvage and primary patency.

66. The method of claim 53, wherein insertion of the scaffold is effective to achieve an improvement of at least 5%, or at least 10%, compared to balloon angioplasty, for at least 3 years after insertion of the scaffold, in the treatment of the target lesion, based on binary restenosis.

67. The method of claim 53, wherein insertion of the scaffold is effective to, relative to balloon angioplasty, reduce the risk of the following for at least 3 years after insertion of the scaffold:
(1) a total occlusion of the target vessel,
(2) a binary restenosis of the target lesion, and
(3) a clinically driven target lesion revascularization (CD-TLR).

68. (canceled)
